(19) United States
(12) Patent Application Publication
(10) Pub. No.: US 2014/0220606 A1
(43) Pub. Date: Aug. 7, 2014

(54) MICROFLUIDIC ASSAY DEVICES AND METHODS

(71) Applicants: Aniruddha Puntambekar, Mason, OH (US); Junhai Kai, Loveland, OH (US); Se Hwan Lee, Cincinnati, OH (US); Jungyoup Han, Cincinnati, OH (US)

(72) Inventors: Aniruddha Puntambekar, Mason, OH (US); Junhai Kai, Loveland, OH (US); Se Hwan Lee, Cincinnati, OH (US); Jungyoup Han, Cincinnati, OH (US)

(21) Appl. No.: 13/981,825

(22) PCT Filed: Jan. 28, 2012
(86) PCT No.: PCT/US12/23052

§ 371 Date: Apr. 17, 2014

Related U.S. Application Data
(60) Provisional application No. 61/437,046, filed on Jan. 28, 2011.

(51) Int. Cl. G01N 33/543; G01N 33/68

(52) U.S. Cl. CPC G01N 33/54393; G01N 33/6869; G01N 33/6866
USPC 435/7.94; 436/501

(57) ABSTRACT

Microfluidic microplate devices and methods for assay systems such as immunoassays, to achieve improvements particularly of higher sensitivity and more repeatable performance, are disclosed. In preferred embodiments, also disclosed are the use of a range of coating buffers for the capture antibody and the use of coating buffers with specific formulations within very narrow ranges to achieve optimal results in the use of the devices and methods.

### CROSS-REFERENCE TO RELATED APPLICATION

This application is a non-provisional application, which claims priority of, and incorporates by reference herein, in part, U.S. Provisional Application No. 61/437,046, filed Jan. 28, 2011.

### FIELD OF THE INVENTION

This invention relates to assay devices and method, for example having application to immunoassays, and more particularly to the integration of microfluidic technology with commonly used microplate architectures to improve the performance of the microplates in the performance of such assays.

### BACKGROUND OF THE INVENTION

Immunoassay techniques are widely used for a variety of applications as described in “Quantitative Immunoassay: A Practical Guide for Assay Establishment, Troubleshooting and Clinical Applications; James Wu; AACC Press; 2000”. The most common immunoassay techniques are 1) non-competitive assay: an example of such is the widely known sandwich immunoassay, wherein two binding agents are used to detect an analyte; and 2) competitive assay: wherein only one binding agent is required to detect an analyte.

In its most basic form, the sandwich immunoassay (assay) can be described as follows: a capture antibody, as a first binding agent, is coated (typically) on a solid-phase support. The capture antibody is selected such that it offers a specific affinity to the analyte and ideally does not react with any other analytes. Following this step, a solution containing the target analyte is introduced over this area whereby the target analyte conjugates with the capture antibody. After washing the excess analyte away, a second detection antibody, as a second binding agent, is added to this area. The detection antibody also offers a specific affinity to the analyte and ideally does not react with any other analytes. Furthermore, the detection antibody is typically “labeled” with a reporter agent. The reporter agent is intended to be detectable by one of many detection techniques such as optical (fluorescence or chemiluminescence or large-area imaging), electrical, magnetic or other means. In the assay sequence, the detection antibody further binds with the analyte-capture antibody complex. After removing the excess detection antibody; finally the reporter agent on the detection antibody is interrogated by means of a suitable technique. In this format, the signal from the reporter agent is proportional to the concentration of the analyte within the sample. In the so called “competitive” assay, a competing reaction between detection antibody and (detection antibody+analyte) conjugate is caused. The analyte, or analyte analogue is directly coated on the solid phase and the amount of detection antibody linking to the solid-phase analyte (or analogue) is proportional to the relative concentrations of the detection antibody and the free analyte in solution. An advantage of the immunoassay technique is the specificity of detection towards the target analyte offered by the use of binding agents.

Note that the above description applies to most common forms of the conventional assay techniques—such as for detection of proteins. Immunoassay techniques can also be used to detect other analytes of interest such as, but not limited to, enzymes, nucleic acids and more. Similar concepts have also been widely applied for other variations as well including in cases; detection of an analyte antibody using a “capture” antigen and a detection analyte.

The 96 well microtiter plate, also referred to herein and commercially as “microplate”, “96 well plate”, “96 well microplate”, has been the workhorse of the biochemical laboratory. Microplates have been used for a wide variety of applications including immunoassay (assay) based detections. Other applications of microplates include use as a medium for storage; for cellular analysis; for compound screening to name a few. The 96 well plate is now ubiquitous in all biochemistry labs and a considerable degree of instrumentation such as automated dispensing systems, automated plate washing systems have been developed. In fact the Society for Biomolecular Sciences (SBS) and American National Standards Institute (ANSI) have published guidelines for certain dimensions of the microplate—and most manufacturers follow them to harmonize the instrumentation systems that can handle these plates. In addition to the basic automated instruments described above, there are numerous examples of specific instrumentation systems developed to improve a specific aspect of the microplate performance. See for instance, U.S. Pat. No. 7,488,451 which discloses a dispensing system for microparticles wherein the system is targeted for loading microparticles in microplates and U.S. Pat. No. 5,234,665 which discloses a method of analyzing the aggregation patterns in a microplate for cellular analysis.

The 96 well platforms, although very well established and commonly accepted suffers from a few notable drawbacks. Each reaction steps requires approximately 50 to 100 microliters of reagent volume; and each incubation step requires approximately 1 to 8 hours of incubation interval to achieve satisfactory response; wherein the incubation time is usually governed by the concentration of the reagent in the particular step. In an attempt to increase the yield per plate, and reduce reaction volumes (and consequently operating cost per plate); researchers have developed increasing density formats such as the 384 and 1536 well microplates. These have the same footprint of a 96 well but with a different well density and well-to-well spacing. For instance, typical 1536 wells require only 2-5 microliters of reagent per assay step. Although offering tremendous savings in reagent volumes, the 1536 well plate suffers from reproducibility issues since the ultra small volume can easily evaporate thereby altering the net concentrations for the assay reactions. 1536 well plate are usually handled by dedicated robotic systems in the so called “High throughput screening” (HTS) approach. In fact, there are innovative examples where researchers have even further extended the plate “density” (i.e. number of wells in the given area) as disclosed in WO05028110B1 wherein an array of ˜6144 wells is created to handles nanoliter sized fluid volumes. This of course, also requires dedicated instrumentation systems. Researchers have invested tremendous energies into modifying microplate architectures; most often within the confines of the SBS/ANSI guidelines; to develop novel designs. One example of this is disclosed in U.S. Pat. Nos. 7,033,819, 6,699,665 and U.S. Pat. No. 6,864,065, wherein a secondary array of micron sized wells is created at the bottom of the well of a conventional 96 well microplate. These miniature wells are used to entrap cells and study their motility patterns amongst other analyses possible with this format.

The next step in miniaturization and automation has been the development of microfluidic systems. Microfluidic systems are ideally suited for assay based reactions, such as disclosed in U.S. Pat. Nos. 6,429,025, 6,620,625 and U.S. Pat. No. 6,881,312. In addition to assay based analysis, microfluidic systems have also been used to study the science of the assays; for example US20080247907A1 and WO2007120515A1 describe methods to study the kinetics of an assay reaction.

Microfluidic systems have also been demonstrated for applications such as cell handling and cellular based analysis as described in U.S. Pat. No. 7,534,331, U.S. Pat. No. 7,326,563 and U.S. Pat. No. 6,900,021, amongst others. The key advantage of microfluidic systems has been their ability to perform massively parallel reactions with high throughput and very low reaction volumes. Examples of this are disclosed in U.S. Pat. No. 7,143,785, U.S. Pat. No. 7,413,712 and U.S. Pat. No. 7,476,363. Instrumentation systems specific for high throughput microfluidics have also been extensively studied and developed, as disclosed in U.S. Pat. No. 6,495,369 and published patent application US20060263241A1. At the same time, a key problem that is still not completely resolved is the issue of world-to-chip interface for microfluidic systems. Researchers have usually developed customized solutions for this problem, on example of which is disclosed in U.S. Pat. No. 6,951,632, depending on the application. This single issue has been a significant bottleneck in widespread adoption of microfluidics. Another problem with widespread adoption of microfluidics has been the lack of standardized platforms. Most often microfluidic devices have specific layout that is well suited for the given application but results in fluidic inlet and outlets positioned at different locations. Indeed, there is little if any commonality even in the footprint or thickness of a microfluidic device that is commonly accepted in the art.

The next logical step in this sequence is naturally the integration of microfluidic systems with the standardized 96, 384 or 1536 well layout. Most often, even though the “microfluidic” microplates use the same footprint as a conventional microplate, the functionality is very specific as disclosed by examples in published application US20060029524A1 and U.S. Pat. No. 7,476,510, for cellular analysis. Researchers have extensively used the standard microplate format as a template to build microfluidic devices. Examples of this abound in the literature as seen by the works of Witek and Park et al., “96-Well Polycarbonate-Based Microfluidic Titer Plate for High-Throughput Purification of DNA and RNA,” Anal. Chem., 2008, 80 (9), pp 3483-3491, and “A titer plate-based polymer microfluidic platform for high throughput nucleic acid purification,” Biomedical Microdevices; Volume 10, Number 1/February, 2008; 21-33; and “A 96-well SPRI reactor in a photo-activated polycarbonate (PPC) microfluidic chip,” Micro Electro Mechanical Systems, 2007. MEMS. IEEE 20th International Conference on, 21-25 Jan. 2007 Page(s):433-436; and the work of Choi et al “A 96-well microplate incorporating a replica molded microfluidic network integrated with photonic crystal biosensors for high throughput kinetic; biomolecular interaction analysis,” Lab Chip, 2007, 7, 1-8, and further in works of Tolan et al., “Merging Microfluidics with Microtitre Technology for More Efficient Drug Discovery,” JALA, Volume 13, Issue 5, Pages 275-279 (October 2008); and even further in work of Joo et al “Development of a microplate reader compatible microfluidic device for enzyme assay,” Sensors and actuators. B. Chemical; 2005, vol. 107, no 2, pp. 980-985. Specifically for cell based assays; a microfluidic design with the same footprint as a microplate is described by Lee et al, “Microfluidic System for Automated Cell-Based Assays,” Journal of the Association for Laboratory Automation, Volume 12, Issue 6, Pages 363-367; and even offered as a commercial product by CellAsic (http://www.cellasic.com/M2.html). All of these are examples of microfluidic devices which are built on the same footprint as of a 96 (or 384) well plate yet do not exploit the full density of the plate.

U.S. Pat. No. 6,742,661 and published patent application US20040229378A1 disclose an exemplary example of the integration of the 96 well architecture with a microfluidic channel network. As described in U.S. Pat. No. 6,742,661 in the preferred embodiment, an array of wells is connected via through-hole ports to a microfluidic circuit. In the preferred embodiment, the microfluidic circuit may be an H or T type diffusion device. U.S. Pat. No. 6,742,661 also describes means for controlling the movement of liquids within this device. The device uses a combination of hydrostatic and capillary forces to accomplish liquid transfer. As explained in greater detail in U.S. Pat. No. 6,742,661, the hydrostatic forces can be controlled by (a) either adding extra thickness to the microplate structure by stacking additional well layers or (b) by supplementing the existing hydrostatic force with external pump driven pressures. U.S. Pat. No. 6,742,661 primarily uses hydrostatic forces (modulated using either of above methods) wherein there is a difference in the hydrostatic forces between the different inlets to a microfluidic circuit. Specifically, the difference in hydrostatic pressure is envisioned as caused by a difference in heights (or depths) of the liquid columns in the wells connected to the different inlets of the microfluidic circuit. The device concepts illustrated in U.S. Pat. No. 6,742,661 are certainly an innovative solution to integrating the Laminar Flow Diffusion Interface (LFDI) type microfluidic devices with a 96 well architecture. However, U.S. Pat. No. 6,742,661 only envisions a self-contained fluidic flow pattern originating from and terminating into wells of the disclosed device. Furthermore, the flow control techniques described in U.S. Pat. No. 6,742,661 fall under the broad category of “pressure driven” flows wherein the hydrostatic pressure of the liquid column controls the flow characteristics. Published patent application US20030049862A1 is another example of attempts to integrate microfluidics with the standard 96 well configuration. It is very important to note that US20030049862A1 defines “microfluidics” in a slightly different manner than conventionally accepted. As defined in US20030049862A1: “Unlike current technologies that position fluidic channels in the fluidic substrate or plate itself, the present invention locates fluidic channels in each of the fluidic modules”. This is achieved by inserting an appropriately sized cylindrical insert into a nominally matching cylindrical well of a microplate. By ensuring a consistent gap between the top surface of the inserted cylinder and the bottom surface of the well, a “microchannel” is defined. Furthermore, the design of the device disclosed in US20030049862A1 is inherently dependent on external flow control; whether by automatic means such as by use of micropumps or by manual means such as be use of a pipette.

Published patent application US20030224531A1 also discloses an example of coupling microfluidics to well structures (including those with standard layouts of 96, 384, 1536 well plates) for electrospray applications. US20030224531A1 uses an array of reagent wells coupled to another array of shallow process zones; of a depth of a micron or even submicron dimensions; wherein the process zones are connected to the reagent wells at one end and to a electrospray emitter tip at the other end. The force for fluidic movement (motive force as defined in US20030224531A1) is provided preferably by an electric potential across the fluid column or also by a pressure differential across the column; which is significant difference from the present invention wherein the fluid movement is purely by capillary forces. The connection to the process zones may be via inlet and outlet microchannels wherein the microchannels are configured to provide additional functionality (such as labeling or purification).

Published patent application WO03089137A1 discloses yet another innovative method for increasing the throughput of a 96 well plate. In this disclosure, the assays are performed within nanometer sized channels within a metal oxide, preferably aluminum oxide, substrate. As disclosed in WO03089137A1, each individual well has a metal oxide membrane substrate attached to the bottom. During operation, each well is individually sealed and a vacuum (or pressure) is applied from a common source, which forces the liquid within the well to be drawn towards the bottom (or away from bottom) of the substrate. Significant improvement in assay performance can be achieved in this method by transporting the assay reagents back and forth through the ultra small openings on the membrane. The innovation described in WO03089137A1 relies on the vacuum and/or pressure source to regulate the transport of liquids within the metal oxide substrate and requires precision pressure control equipment to achieve optimum performance.

Published patent application US20090123336A1 discloses the use of an array of microchannels connected to a series of wells wherein the wells are in the format of a 384 well plate. As described in US20090123336A1, a loading well serves as a common inlet for multiple detection chambers each of which is positioned in the location of a “well” on a 384 well plate. Importantly, US20090123336A1 is limited to the use of multiple detection chambers connected to a single loading point owing to challenges in making microfluidic interconnects to the high density microfluidic channel network; which if not impossible is extremely difficult. This imposes limitations on the methods of use for the invention of US20090123336A1, which requires specialized handling steps to perform unique arrays in each of the serially connected chambers. Specifically, as disclosed in US20090123336A1, the only way to perform unique assays in the serially connected chambers is to deposit the capture antibody on the channel surface prior to sealing the channel surface. This step, in itself, would require sophisticated dispensing systems to accurately (a) deliver desired liquid volume at (b) precisely defined locations; thereby adding to the overall cost of the system. In other embodiments of this disclosure, a common solution is sucked into the array of serially connected channels by dipping one end of the channel path in the liquid solution. The inventors also claim that “when a common loading channel is present, reagents can be simultaneously loaded into all channels by capillary forces or a pressure difference . . . ”. Although theoretically correct, it is well known in the art of microfluidics that is virtually impossible to govern flow in multiple branching channels via a single source. There will always be preferentially higher flow rate in at least one of the branching channels which implies variations in an assay performed across multiple such channels.

It will be appreciated from the following description of preferred embodiments of the invention, that the present invention is particularly suitable for point-of-care test (POCT) applications. For POCT applications it is frequently desired to use an immunoassay based test approach that can detect across an extended dynamic range for applications such as the ones described above. The most common technique for testing at the POC is by use of the so called “Lateral Flow Assay” (LFA) technology. Examples of LFA technology are described in U.S. Pat. Nos. 5,710,005 and 7,491,551, and published patent applications US20060051237A1 and WO2008122796A1. A particularly innovative technique for LFA is also described in published patent application WO2008049083A2, which employs commonly available paper as a substrate and wherein the flow paths are defined by photolithographic patterning of non-permeable (aqueous) boundaries. Advances in LFA technology are disclosed in disclosures such as published patent application US20060292700A1, wherein a diffusive pad is used to improve the uniformity of the conjugation thereby providing improvements in assay performance. Other disclosures such as in published patent applications WO9113998A1, WO03004160A1, and US20060137434A1, have used the so-called “microfluidic” technology to develop more advanced LFA devices. Microfluidic LFA devices are generally considered to have better repeatability than membrane (or porous pads) based LFA devices, owing to the precision available in these devices in the fabrication of microchannel or microchannels+precise flow resistance patterns. In some cases, devices such as those disclosed in published patent application US20070042427A1 combine commonly used technologies in both the microfluidics and LFA arts. As disclosed in US20070042427A1, the flow is initiated by a bellows type pump and thereafter maintained by an absorbent pad.

### SUMMARY OF THE INVENTION

Hence the present invention seeks to address the shortcomings of the above-described art and seeks to provide an easy and reliable system that integrates the advantages of microfluidic technology with the standardized platforms of microplate platforms. The apparatus and techniques contemplated by this invention are also novel in that a “microfluidic microplate” in accordance with the present invention is completely compatible with all of the currently available conventional commercial instrumentation designed for similarly sized conventional microplates.

For purposes of this disclosure of the invention as set forth herein, the contents of published PCT Patent Application No. PCT/US2010/042506, commonly assigned herewith, are hereby incorporated herein by reference in their entirety, to the extent any portion of the subject matter of such contents is not described in particularity herein.

In one aspect, the present invention involves an improved method for performing an immunoassay or group of immunoassays on a sample on a selected microfluidic microplate, wherein a priming buffer is used as the first reagent in the immunoassay sequence, the improvement wherein the priming buffer is a liquid with lower surface tension than the surface tension of water.

In another aspect, the invention involves a method for increasing the sensitivity of immunoassays performed using microfluidic microplates, which method comprises using suitably high concentrations of capture and/or detection antibodies as compared to the concentrations of capture and/or detection antibody concentrations, and wherein the capture and/or detection antibody concentration is greater than and up to at least 20 times higher than the concentration of the capture and/or detection antibody used for the same assay on a conventional 96-well microplate.

Other aspects of the invention, and the objectives and advantages thereof, will be apparent to those skilled in the art from the following description.

### BRIEF DESCRIPTION OF THE DRAWINGS

FIG. 1 shows a top view of an embodiment of the present invention wherein an array of 96 wells is connected via through holes to 96 individual microchannels. The microplate array shown matches the dimensions of conventional microplates (as defined by accepted ANSI standards). The positions of the wells also matches ANSI standards. Each well is connected to a microchannel on the opposing face of the substrate. FIG. 1 does not show sealing layer (for microchannels) and absorbent pad for clarity. Also, selected wells in lower right hand corner of top figure do not show microchannel pattern for ease of explanation.

FIG. 2 shows a cross sectional view of a portion of an embodiment of the present invention illustrating the relative positions of the well structure, microchannel structure, sealing layer and absorbent pad. Each well is connected to a microchannel on the opposing face of the substrate. Microchannels are sealed by a sealing layer which in turn has an opening. Opening on sealing layer connects on other side to an absorbent pad. When liquid is introduced in the well, it is drawn into the microchannels by capillary force; the liquid travels along the microchannel until it reaches the opening in the tape. Thereupon, liquid front contacts the absorbent pad which exerts stronger capillary force and draws liquid until well is emptied. Depending on the interface configuration at well-microchannel interface; the liquid will also be emptied from the microchannel or liquid motion will stop when rear end of liquid column reaches well-microchannel interface. In latter case, the liquid is still filled in the microchannel.

FIG. 3 shows a 3-dimensional illustration of an embodiment of the present invention with details of the parts that constitute the microfluidic microplate and the associated holder. FIG. 3A shows the basic constituents of the microfluidic microplate: the substrate layer, sealing layer and absorbent pad. FIG. 3B shows the use of the microfluidic microplate with a suitable holder. Insert images at bottom show close up views of the substrate layer showing the well, through hole and microchannel structures.

FIG. 4: FIG. 4A shows a preferred embodiment of the present invention wherein the through hole connecting the well and the microchannel conforms to certain rules. In the preferred embodiment shown, the width of the hole (w) is be greater than, and at least equal to, the depth (d) of the hole. This ensures that when liquid is introduced in the well, the front meniscus of the liquid can “dip” and touch the surface of the sealing tape. The meniscus also touches all 4 “walls” of the microchannel connected to one part of the hole (left hand side in above figure). Thereafter, capillary forces will draw the liquid from the well and fill the microchannel. FIG. 4B shows an alternative aspect of the present invention wherein the through hole connecting the well and microchannel contains a tapered section feature of the interface between the well structure and the microchannel. Preferably, the width of the through hole at the top (w) is be greater than, and at least equal to, the depth (d) of the hole; and furthermore the through-hole also has tapered walls. The taper angle (with respect to horizontal) of the walls of the through hole is greater than or equal to the taper angle on the walls at the base of the well structure immediately preceding the through hole. This ensures that when liquid is introduced in the well, the front meniscus of the liquid can “dip” and touch the surface of the sealing tape. The meniscus also touches all 4 “walls” of the microchannel connected to one part of the hole (left hand side in above figure). Thereafter, capillary forces will draw the liquid from the well and fill the microchannel.

FIG. 5 shows different configurations of the microchannel section of a preferred embodiment of the invention connecting to the through hole at the bottom of the well. In FIG. 5A there is an abrupt transition from the cross sectional area of the through hole to the cross sectional area of the microchannel. Since the cross sectional area of the channel is much smaller; the liquid exiting the well will stop at the interface. In FIG. 5B the microchannel is slightly larger than the interface hole and furthermore, the channel cross section gradually tapers to the final dimension. In this case, as the liquid exits the well, it will continue to flow (into absorbent pad) until even the microchannel is completely emptied.

FIG. 6 shows an aspect of the present invention wherein a air-vent is incorporated in the flow path. A three-dimensional schematic illustration of the air-vent feature is shown, to ensure ability to “stop” flow when the liquid has emptied from the well but is still occupying the microchannel. The air vent allows the “negative pressure” (i.e. capillary suction force) of the absorbent pad to equilibrate with atmospheric pressure to ensure there is no pressure differential across the liquid column in the microchannel. Conventional sealing tape and pad are not shown for clarity. In this embodiment, the air vent is slightly offset from the outlet (outlet at end of microchannel towards right of air vent in above images). Liquid is loaded in the well, drawn in the channel by capillary force and transported to the pad. After the well is completely emptied, the front end of the liquid column is still in contact with the absorbent pad which will continue to draw more liquid. The liquid will then retract back into the channel (from front end), wherein using embodiments shown in FIG. 8 ensure liquid always retracts from outlet, until the liquid crosses the air vent hole. As the liquid retracts from the outlet hole (on tape), flow continues because of thin film of liquid in the two corners where the channel is sealed by hydrophilic tape. As the liquid crosses the air vent, the capillary suction force of the pad is equilibrated by the atmospheric pressure from the air vent causing flow to stop.

FIG. 7: FIG. 7A and FIG. 7B show embodiments of the microchannel pattern of a device in accordance with preferred embodiments of the present invention. The channel pattern can be serpentine as shown in FIG. 7A. The channel pattern can further be modified such that there is a continuous taper in the channel path from the interface hole to the absorbent pad. The taper ensures that there is increasing capillary force on the front end of the liquid column and result in a different flow rate than in the case when the channel is not tapered.

FIG. 8: FIG. 8A and FIG. 8B show yet other configurations of the channel of preferred embodiments of the invention. Composite channel geometries are incorporated therein to ensure that as liquid loss (due to evaporation) occurs, the liquid column will always retract from the outlet and the inlet position of the liquid column shall be maintained at the interface of the through-hole and the microchannel. Note that the values for the initial and end section are for illustrating the difference with the remainder of the microchannel and are not limited to stated values.

FIG. 9 shows other preferred embodiments of the invention with differing channels and effect of these embodiments on flow rate and evaporation rate in the practice of the present invention, including use of composite geometry in the microfluidic channel part of the microfluidic microplate. The different colors in the spiral microfluidic channel figure represent different dimensions tested. As shown in the accompanying Table, increasing the end section size allows for high flow rate and significantly long times for loss of liquid in last loop due to evaporation. Note that when a larger end section is used the capillary forces at the inlet (at through hole interface) are higher ensuring that the liquid never “moves” from the inlet end during incubation periods. Also as shown in the Table, using a smaller dimension for the initial section allows for greatly reduced flow rates (longer residence times for liquid as it is flowing through the channel). The smaller initial section also exerts a higher capillary force ensuring liquid does not move from inlet end during incubation.

FIG. 10 shows embodiments of the invention using polymer beads to increase sensitivity. In FIG. 10A, the microchannel is packed with microbeads; thereby even further increasing the surface area to volume ratio within the microchannel. In FIG. 10B, the beads are packed only in the through hole structure and a simple channel is used to draw the liquid away from the vertical line of sight of the through hole. In the embodiment of FIG. 10B, the packed bead array within the through hole acts as the reaction chamber. The through hole dimensions can be adjusted (since constraints described in FIG. 4 no longer apply) to tune sensitivity.

FIG. 11 shows a preferred embodiment of the invention suitable for handling cells in the microfluidic microplate. An array of pillars is fabricated in the channel path. If a solution containing cells is introduced in the well, it will be drawn into the microchannel and the solution will pass through while the cells will be trapped at the pillar array.

FIG. 12 shows even yet another embodiment of the channel in a preferred embodiment of the invention. In this embodiment, the microchannels are fabricated on a separate layer from the layer containing the well array. The microchannel path extends on both faces of the substrate containing the microchannels and furthermore channels from opposing face are connected via an additional through hole on the channel substrate. This greatly extends the channel length and consequently total surface area and volume for reactions.

FIG. 13 shows a preferred embodiment of the invention wherein a unique absorbent pad is connected to each microchannel. In this embodiment, a separate absorption pad is used for each of the 96 wells. Furthermore, the absorption pads are not physically attached to the microplate; instead they are attached to a base layer over which the microplate is positioned for operation. Furthermore, the pads are not in the same vertical line-of-sight as the wells and the microchannels. For ease of explanation, only one row of absorption pads is shown; whereas the entire microplate device would contain 96 distinct pads.

FIG. 14: FIG. 14A and FIG. 14B show cross sectional views of a device in accordance with further preferred embodiments of the invention showing the effects of compressing the absorbent pad. FIG. 14C shows an alternate embodiment to ensure reliable contact between the absorbent pad and the microfluidic channel by use of protrusion structures. FIG. 14A shows that when the absorbent pad is attached to the microplate (for example by using adhesives) the interface at the sealing tape is reasonably flat. When the absorbent pad is compressed by a base layer, the pad bulges into the hole at the sealing tape and is in close proximity (or makes physical contact) with the enclosed microchannel cross-section; as shown in FIG. 14B. The latter ensures that the liquid easily contacts the absorbent layer. The absorbent pads can also be mounted on the base layer. In FIG. 14C, the top illustration shows a schematic illustrating the protrusion structure at the end of the microfluidic channel, and at the bottom (series of 6 images) is shown three-dimensional illustrations of different embodiments of the protrusion structure and the end of the microfluidic channel. Please note that in these images; the protrusion is directed upwards whereas the schematic illustration on the top shows it directed towards the bottom.

FIG. 15 shows a preferred embodiment of the invention illustrating an absorbent pad layout wherein an absorbent pad is common to a row or column of microchannels. In this embodiment, the absorbent pads are configured as strips that are connected to each column (or each row). Furthermore, the pads are not in the same vertical line-of-sight as the wells and the microchannels. For ease of illustration, only two columns of absorbent pads are shown; whereas the entire microplate device would contain 12 distinct columns (or 8 distinct rows).

FIG. 16 shows an alternate embodiment for the absorbent pad layout similar to that illustrated in FIG. 15, wherein an absorbent pad is common to a row or column of microchannels, and furthermore wherein the absorbent pad is on the opposite side of the substrate as the microchannels.

FIG. 17 shows a further preferred embodiment of the invention wherein an additional section of the microchannel is used as capillary pump and waste reservoir to replace the absorbent pad. In this embodiment, the microfluidic microplate uses an additional length of the microchannel as the capillary pump to replace the absorbent pad, reducing the plate to only two layers: the substrate (with well, through-hole, and microchannel) and the sealing layer. The amount of total liquid that can be added per well in this embodiment is limited by the volume of the “waste” channel section.

FIG. 18 shows another preferred embodiment of the device in accordance with the invention wherein a microfluidic insert plate is used instead of a single continuous substrate. In this embodiment, the microfluidic channels and wells are fabricated on a separate substrate which is then assembled with an enclosure matching the shape of a conventional 96 well plate. The (well+microchannel) plate is only positioned by the enclosure and can be removed from the enclosure. As shown in FIG. 18A, initially the plate is oriented such that the wells are facing upwards and solutions can be added to the wells. As shown in FIG. 18B, after all solutions have been added, the plate can be removed from the enclosure, flipped over and mounted such that the wells are facing downward with the channels facing upwards. This allows the channels to be in closer proximity to the detection system. For ease of explanation only 1 pipette dispensing and 1 photodiode are shown.

FIG. 19 shows yet another preferred embodiment of the invention wherein a microfluidic insert plate is used instead of a single continuous substrate and an additional layer is used to minimize optical cross-talk during detection. In this embodiment, the microfluidic channels and wells are fabricated on a separate substrate which is then assembled with an enclosure matching the shape of a conventional 96 well plate. The (well+microchannel) plate is only positioned by the enclosure and can be removed from the enclosure. Initially the plate is oriented such that the wells are facing upwards and solutions can be added to the wells (not shown). After all solutions have been added, the plate can be removed from the enclosure, flipped over and mounted such that the wells are facing downward with the channels facing upwards. Furthermore, yet another layer is added such that the additional layer has well structures matching the footprint of the microchannels. The additional structure shall preferably be made from a opaque material to minimize cross-talk during detection. For ease of explanation only 2 photodiodes are shown.

FIG. 20 shows a preferred embodiment in accordance with the invention similar to the one in FIG. 18 except that multiple microfluidic insert plates are used. In this configuration, the microfluidic channels and wells are fabricated on multiple separate substrate which are then positioned onto an enclosure matching the shape of a conventional 96 well plate. In FIG. 20, one embodiment is shown wherein each of the individual substrate is approximately the size of a conventional glass plate (approximately 25 mm×75 mm).

FIG. 21: FIG. 21A shows an embodiment in accordance with the invention wherein multiple microfluidic reaction chambers are serially connected to a common loading well. In this embodiment, one loading well is connected to a microchannel path on the opposing surface and is additionally connected to other microchannel sections not directly underneath the loading well. When a liquid is loaded into the well, it will flow through all the microchannel sections (4 sections) connected in series. A total of 24 distinct loading wells connect to a total of 4 reaction chambers each. The microchannel can be modified to accommodate the series connection of the channel sections.

FIG. 21B shows an embodiment in accordance with the invention wherein the loading well and the microfluidic reaction chamber are not in the same vertical line of sight. In this embodiment, one loading well is connected a unique microchannel, but the microchannel and the loading well are offset in the vertical line of sight. The microfluidic reaction chamber occupies the “well” position of another “well” as defined in the conventional 96-well microplate layout. This configuration allows for the use of a simplified geometry well (as shown in insert three-dimensional schematic) which couples to an in-out spiral microfluidic reaction chamber. FIGS. 21C and 21D show additional embodiments in accordance with the invention wherein multiple loading wells are connected to a single microfluidic reaction chamber for a “semi-auto” microfluidic microplate.

FIG. 22 shows a preferred embodiment in accordance with the invention particularly well suited for low flow rates for an extended period of time. In this embodiment the microplate is mounted in a fixture specially configured for use in conjunction with this embodiment. The fixture is connected to an air pump that can pump air at room temperature or elevated temperatures through the fixture which passes on the underside of the absorbent pad. The flow sequence is configured such that prior to the step where a low, steady flow rate for a extended duration is desired, a high volume of liquid is added to completely saturate the pad such that it cannot absorb any further liquid. Then the desired liquid is added to the wells and the wells are sealed on top to prevent evaporative loss. Furthermore, air flow is initiated in the fixture which will cause evaporative loss of liquid from the pad. As the pad loses liquid volume, additional liquid volume will be drawn from the wells at a low flow rate for an extended period of time. The absorbent pad may be a common pad for all wells or separate pads for each well.

FIG. 23 shows a preferred embodiment of the invention particularly suitable for chemiluminescence based detection assays. In this embodiment, each cell (wherein a cell composes of one well with a through-hole connecting to a single microchannel) is physically isolated from the adjoining cells by a substantially opaque substrate. The “microfluidic substrate” is, in actuality, an array of physically distinct substrates.

FIG. 24 shows preferred embodiments of the present invention adapted for a completely manual point-of-care (POC) assay test. Reduced and simplified versions of the microfluidic microplate of the invention are shown to illustrate this embodiment: on the left is shown a configuration (substantially identical to the microfluidic microplate cells) for the POC device wherein the microchannels are positioned below the loading wells, and on the right is shown an alternate embodiment; wherein the microchannels are positioned at a different location from the loading well.

FIG. 25 shows images of the microfluidic microplate in accordance with the present invention suitable for performance of the novel assays of the invention.

FIG. 26 shows images of another microfluidic microplate in accordance with the present invention suitable for performance of the novel assays of the invention. The image on the bottom shows the microfluidic microplate being positioned in a holder for liquid handling steps—in actual operation; the holder also houses the absorbent pad in the embodiments wherein the pad is a disposable element.

FIG. 27 shows a method of using conventional standard or narrow end pipette tips to dispense into the microfluidic microplate in accordance with the present invention suitable for performance of the novel assays of the invention. Note that the microchannels are not shown in this illustration.

### DETAILED DESCRIPTION OF THE INVENTION

Therefore, this invention, in use, contemplates improvements in assay devices and methods using a so-called “microfluidic microplate” also called the “μF96” or “μf96” such as the “Optimiser™” or the “microfluidic microplate” wherein a microfluidic channel is integrated with a well structure of a conventional microplate. The present invention is particularly useful in conjunction with a means of integrating microfluidic channels with an array of wells on a platform conforming to the standards of the SBS/ANSI. Thus, this invention presents the following advantages, in use, for example in conjunction with the Optimiser™ plate, commercially available from Siloam Biosciences, Inc., Forest Park, Ohio, which may be used in multiple applications to replace conventional microplates.

Accordingly, some advantages of use of the present invention include, but are not limited to:
1. The μf96 (or herein also referred to as the “Optimiser™”) plate combines the speed and versatility of microfluidic approach with the well established 96 well platform
2. As far as the user is concerned; the operation is exactly identical to a conventional 96 well plate in fact with a reduced number of steps
3. The μf96 (or Optimiser™) plate can potentially significantly reduce reagent consumption and/or sample requirement. For relatively high abundance samples; sample volume as low as 0.4 μl may be sufficient (for 50 μm spiral channel). This is also important for using lower amounts of reagents—e.g. antibodies in an immunoassay application.
4. The μf96 (or Optimiser™) plate can be significantly faster than a conventional 96 well plate in applications such as immunoassays. A full set of 96 assays can be potentially completed in 5-30 minutes as opposed to hours on a regular 96 well plate.
5. The cost of a μf96 (or Optimiser™) plate can be comparable to a conventional plate since it also a single injection molding operation. The slight added costs due to (a) microfabricated master mold on one side; and (b) pad layer will be well offset by the lower reagent consumption and faster analysis times
6. The basic approach is extremely versatile and lends itself to a wide variety of applications not only in a lab setting but also for point-of-care test devices.
7. Since the flow is governed only by geometric and material effects, there is reduced operator error which will lead to more reproducible results
8. Just like a 96 well plate, the μf96 (or Optimiser™) plate operation can also be fully automated. In fact the μf96 (or Optimiser™) would only require a plate handling and robotic reagent dispensing system. Compared to a 96 well plate which requires (i) plate handling system, (ii) robotic reagent dispensing system; (iii) incubation system (owing to long incubation times); and (iv) plate washing system; this is a much reduced instrument load for full automation.
9. The sensitivity of the Optimiser™ can be easily “tuned” to meet the specific needs for a given assay.
10. The Optimiser™ can in fact be used to “optimize” assay performance across parameters of speed, sensitivity and sample volume allowing end-users extraordinary flexibility in immunoassay based analysis.

The overall microplate dimensions and layout of wells matches those of the 96 or 384 or 1536 well formats prescribed by the SBS/ANSI standards. The microfluidic microplate consists of an array of wells defined on one face of a substrate. Each well is connected to a microfluidic channel on the opposing face of the substrate via a suitable through hole at the bottom of the well. The microfluidic channels are in turn sealed by an additional sealing layer which has an opening at one end (outlet) of the microchannel. Furthermore, the sealing layer is in contact with an absorbent pad.

When a liquid is introduced in the well, it is drawn into the microchannel by capillary forces. The liquid travels along the microchannel until it reaches the absorbent pad. The absorbent pad exerts stronger capillary forces than the microchannel and draws the liquid out of the channel. By suitable design, it can be ensured that as the liquid exits the well and flows into the absorbent pad; the rear end of the liquid “sticks” at the interface between the well and the microchannel. At this stage, the well is completely emptied of the liquid whereas the channel is still filled with the liquid. When a second liquid is now added to the well, the capillary barrier holding the first liquid is broken and the capillary action of the pad is re-started and the second liquid is also drawn via the channel into the pad. This sequence can be repeated a number of times to complete an immunoassay sequence. Thus, the device of this invention allows for a microfluidic immunoassay sequence on a microplate platform. Furthermore, the method of using the plate is exactly identical to a conventional microplate and the device of the present invention is also compatible with the appropriate automation equipment developed for the conventional microplates. Other embodiments of the device of the present invention can be used for applications such as cell based analysis.

As referenced herein and as indicated above, μF96 or μf96, or the Optimiser™, refer to a 96 well microfluidic microplate wherein each well is connected to at least one microfluidic channel. Unless otherwise explicitly described, the microfluidic microplate shall be assumed to be made of 3 functional layers, namely the substrate layer (with the wells, through-hole structures and microchannels), the sealing tape layer, and an absorbent pad layer; wherein the “96” refers to a 96 well layout and similarly μf384 would refer to a 384 well layout and so forth. As used herein, the term Optimiser™ is also used to describe the present invention and similarly, Optimiser™-96 shall refer to a 96 well layout, Optimiser™-384 shall refer to a 384 well layout and so forth. Furthermore, “microchannel” and “microfluidic channel” and “channel” as used herein all refer to the same fluidic structure unless otherwise dictated by the context. The term “interface hole” or “through hole” or “via hole” all refer herein to the same structure connecting the well structure to the microchannel structure unless dictated otherwise by the context. The term “cell” is used herein to describe a functional unit of the microfluidic microplate wherein the microfluidic microplate contains multiple essentially identically “cells” to comprise the entire microplate.

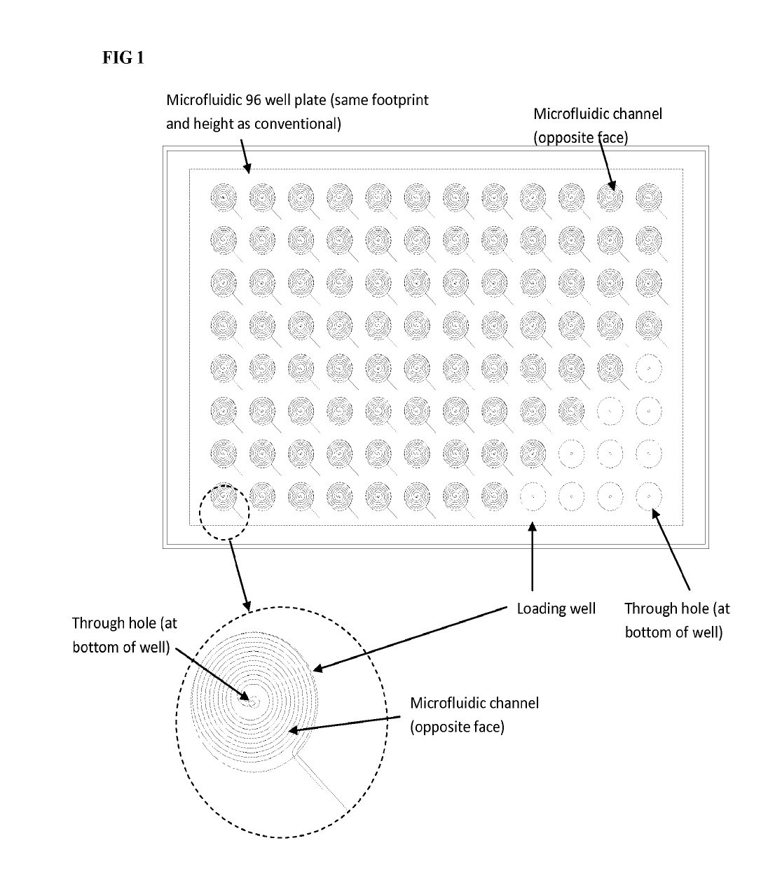

The present invention can be readily understood by examining the figures of the attached drawings. The basic concept can be understood by reviewing FIG. 1 and FIG. 2 and FIG. 3. FIG. 1 shows the top of view of a microfluidic 96 well plate or the microfluidic microplate. The plate matches the dimensions of conventional microplates (as defined by accepted ANSI standards). The positions of the wells also match ANSI standards. Each well is connected to a microchannel on the opposing face of the substrate. In the embodiment shown in FIG. 1, the wells and the microchannels are fabricated on the same substrate layer. A noteworthy feature of the present invention is understood from FIG. 1; wherein the loading position (for adding liquid reagents) and the detection region are in the same vertical plane; which matches the conventional microplate exactly.

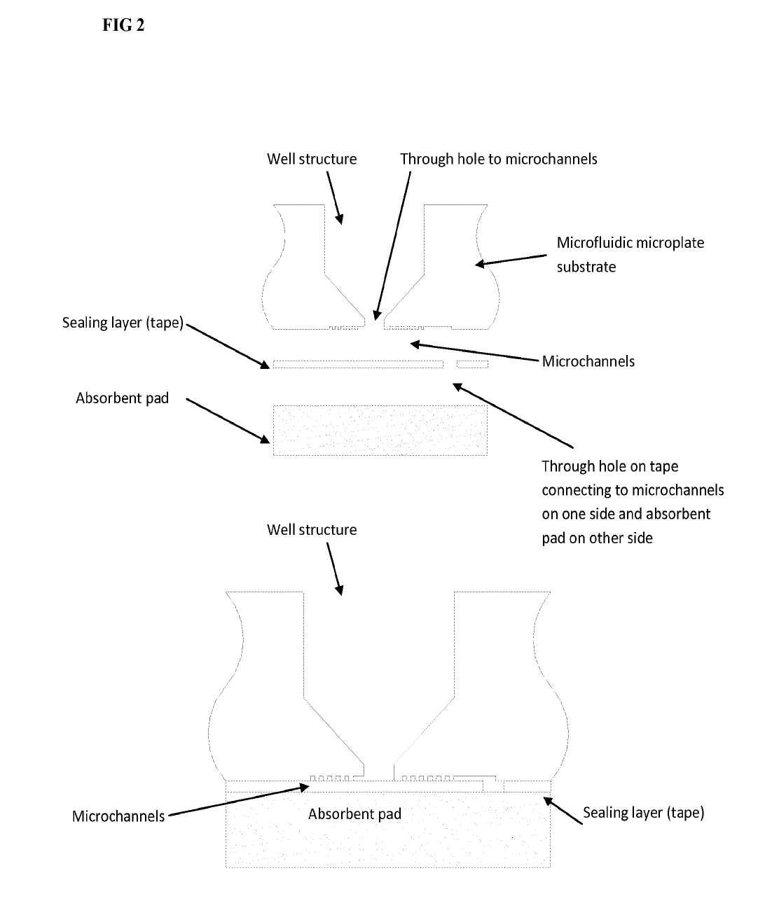

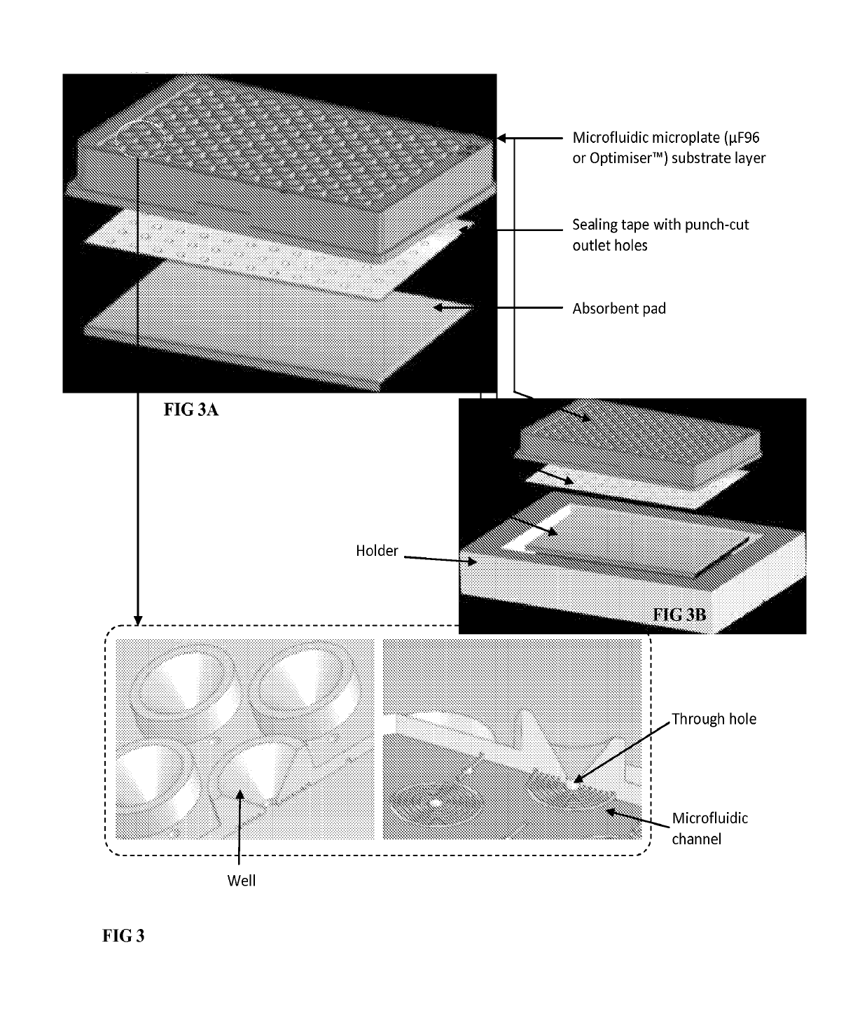

FIG. 2 shows cross-sectional views of a portion of the microplate showing 1 unit of 96 in exploded and assembled views. FIG. 3A shows three-dimensional (3D) view of the microplate, sealing layer, and absorbent pad in exploded view. FIG. 3B shows 3-dimensional view of the microplate, sealing layer, absorbent pad, and a holder in exploded view. Each well is connected to a microchannel on the opposing face of the substrate. Microchannels are sealed by a sealing layer which in turn has an opening at the other end of the microchannel (as compared to the on end connected to the through hole at the bottom of the well). Opening on sealing layer connects on the other side to an absorbent pad. In the preferred embodiments, an array of absorbent pads are used such that the absorbent pads are not in the same vertical line of sight as the loading well and the channels. Alternately, as shown in FIG. 3, the absorbent pad can be a single continuous piece connected to all the 96 microchannel outlets. When liquid is introduced in the well, it is drawn into the microchannels by capillary force; the liquid travels along the microchannel until it reaches the opening in the tape. Thereupon, liquid front contacts the absorbent pad which exerts stronger capillary force and draws liquid until well is emptied. In the preferred embodiment, the through hole, microfluidic structure and absorbent pad are configured such that as the liquid exits the well the rear end of the liquid column cannot move past the interface between the through hole and the microchannel. Consequently, the well is completely emptied of its liquid contents and the liquid is partially absorbed by the absorbent pad whereas a portion of the liquid still occupies the complete microfluidic channel. This configuration can be used as an incubation step for immunoassay based analysis.

When a second liquid is added to the well, the second liquid makes contact with the rear end of the first liquid at the interface of the through hole and the microchannel. At this stage, there is again a continuous liquid column from the absorbent pad extending via the microchannel and the through hole to the well. The lower surface tension of the liquid column filling the well will cause flow to resume and the first liquid will be completely drawn out of the channel and replaced by the second liquid. The second liquid will also be drawn out of the channel until the rear end of the second liquid now reaches the interface between the through hole and the microchannel where the flow will stop again. This sequence is continued until all steps required for an immunoassay are completed. This also illustrates a particularly advantageous aspect of the present invention—namely the fact that the sequence of operation only involves liquid addition steps. There is no need to remove the liquid from the well since it is automatically drained out. This considerably reduces the number of steps required for operation and simplifies the operation of the microfluidic microplate. Also, as described earlier, in the preferred embodiment the absorbent pads are positioned such that the pads are not in the same vertical line of sight as the reaction chambers. In this scheme the pads can be integral to the microfluidic microplate; whereas if desired, the pads can be configured to a removable component that can be discarded after the last liquid loading step, for example in the case of the embodiment shown in FIG. 3.

In preferred embodiments of the present invention, the substrate containing the well, through hole and microchannel is transparent. This allows for optical monitoring of the signal from the microchannel from the top as well as bottom of the microplate; a feature that is common on a wide variety of microplate readers used in the art. In other embodiments, the substrate may be an opaque material such that the optical signal from the microchannel can only be read from the face containing the channel. For example, in the embodiment shown in FIG. 2, the signal can only be read from the “bottom” if the substrate were an opaque material. As described later, yet another method could use rotation of an insert layer to allow for top reading with an opaque substrate material.

The microfluidic microplate can be manufactured by a conventional injection molding process and all commonly used thermoplastics suitable for injection molding may be used as a substrate material for the microfluidic microplate. In a preferred embodiment, the microfluidic microplate is made from a Polystyrene material which is well known in the art as a suitable material for microplates. In other preferred embodiments, the microfluidic microplate is made from Cyclic Olefin Copolymer (COC) or Cyclic Olefin Polymer (COP) materials which are known in the art to exhibit a lower auto-fluorescence and consequently lower background noise in fluorescence or absorbance based detection applications.

An example assay sequence for a sandwich immunoassay performed using the devices and methods provided by the present invention is described in the following text. By using well known techniques in the art, a wide variety of such assays can be performed on the microfluidic microplate. As is readily evident from the description of the invention herein and as will be appreciated by those skilled in the art, all of the reagent addition steps can be performed by automation systems designed to handle liquids for current microplate formats, substantially without any changes.

In operation of the invention, in a preferred embodiment, the following sequence can occur:
1. To cause a flow sequence; the first liquid is pipetted into the well.
2. The volume of the liquid loaded into the well should be at least slightly larger than the internal volume of the channel.
3. The liquid will be drawn into the microfluidic channel and will continue to move due to capillary force.
4. The liquid will flow from the well via the channel till it reaches the outlet where it will touch the absorbent pad.
5. After this, the absorbent pad will continue to draw the liquid till all the liquid in the well is emptied into the channel and then into the pad. The liquid flow will stop when the rear end of the liquid column reaches the interface between the through hole at the base of the well and the channel.
6. The flow rate in this configuration is completely controlled by (a) liquid type; (b) geometries of well and channel and interface ports (namely the through hole) (c) material properties of the μf96 (or Optimiser™) microplate; specifically surface properties; and (d) absorbing characteristic of the pad.
   a. The flow rate can be manipulated by varying any one of the parameters.
   b. The initial “filling” flow rate is independent of the pad and is based only on channel properties
   c. Thereafter the channel acts as a fixed resistance (except at the very end when the liquid is emptying) and the pad acts as a vacuum (or capillary suction) source.
   d. If desired, the assay steps can be under static incubation to ensure that there is minimal effect of flow rate variation on assay response.
7. After this a second liquid may be added and the same sequence can be repeated.
   a. Alternately, the second liquid can be loaded just as the first liquid is emptying from the well. This will lead to a continuous liquid column without a stop in flow between the first and second liquid.
8. After the last liquid that should be added is passed through the system, the absorbent pad(s) may be removed if desired. The lack of further capillary force will guarantee a stop to the liquid motion.
9. The plate can be read from the top of the well or from the bottom side or if the well structure interferes with optical signals, the μf96 (or Optimiser™) may be flipped over and read from the channel side. If the latter is required, the plate configuration should be modified such that the plate still fits a standard holder for SBS/ANSI 96 well plates.

Resulting assay, example:
1. Add capture antibody and flow—capture antibody will non-specifically adsorb on channel surface. Repeated injections of capture antibody solution can potentially increase concentration on surface.
2. Wait till the capture antibody solution is completely sucked through the well. The capture antibody solution is still completely filling the microchannel. Incubate to allow capture antibody conjugation to channel surface.
3. Add blocking buffer and flow; incubate to allow blocking media to conjugate to remaining channel surface.
4. Add sample and flow; incubate to allow target analyte to link with capture antibody
   a. Optionally, repeated injections of sample can increase detection sensitivity
5. (Optional) flush again
6. Add labeled detection antibody and flow; incubate to allow detection antibody to conjugate to captured target analyte
7. Flush with buffer
8. For Fluorescence based assay, the plate can now be transferred to reader
9. For luminescence of chemifluorescence assay—add substrate which will fill channel and allow it to incubate
10. For luminescence or chemifluorescence assay, the plate can now be transferred to a suitable reader

The well structure shown in FIG. 2 comprises a straight (cylindrical) section and a tapering (conical) section. The taper allows for complete flushing out of well contents as opposed to having a small through at the base of a cylindrical well structure. It will be appreciated that a wide variety of configurations are possible for this basic scheme; for instance when the through hole is not at the center of the well but offset to one side; or wherein the microchannel pattern is of different configuration; or wherein the absorbent pad is placed in a different position; or wherein the relative depth and/or position of the well structure and microchannel with respect to total plate thickness (set as 14.35 mm by SBS/ANSI standards) is varied. Indeed, although highly desirable for standardization, the Optimiser™ microfluidic microplate can also be made to dimensions NOT confirming to the ANSI/SBS specs in certain examples. A few of these are described as examples of embodiments possible with this concept. The embodiments described herein are merely to illustrate the flexibility of this concept and are not intended to limit the present invention.

One embodiment is shown in the 3-dimensional (3D) view of FIG. 3. As shown in FIG. 3 insert, the well does not have a “straight” section at the top, but only a taper section. This minimizes the potential for any residue at the transition point from the vertical wall to the tapered wall of the well. Also, as shown in FIG. 2 and FIG. 3, the well may be configured such that the substrate completely surrounds the well or the surrounding substrate may be created in the form of “lip” structure. The latter minimizes the amount of polymer material required for the part thereby reducing cost. The use of the “lip” structure also makes the part more amenable to injection molding operations since the lower amount of material in this configuration exhibits less shrink during the molding process; which is advantageous since said shrink may cause distortion of the well, through hole and microchannel patterns.

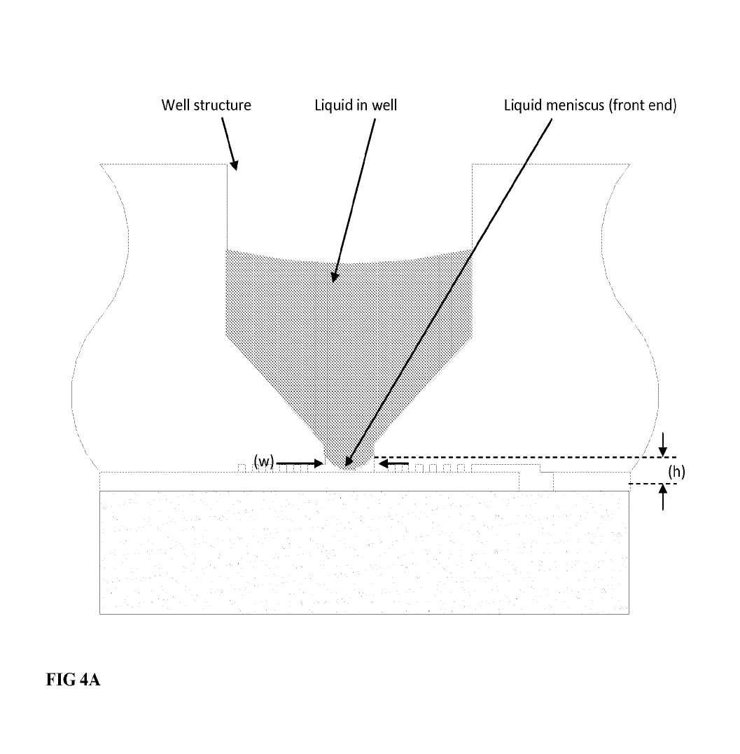

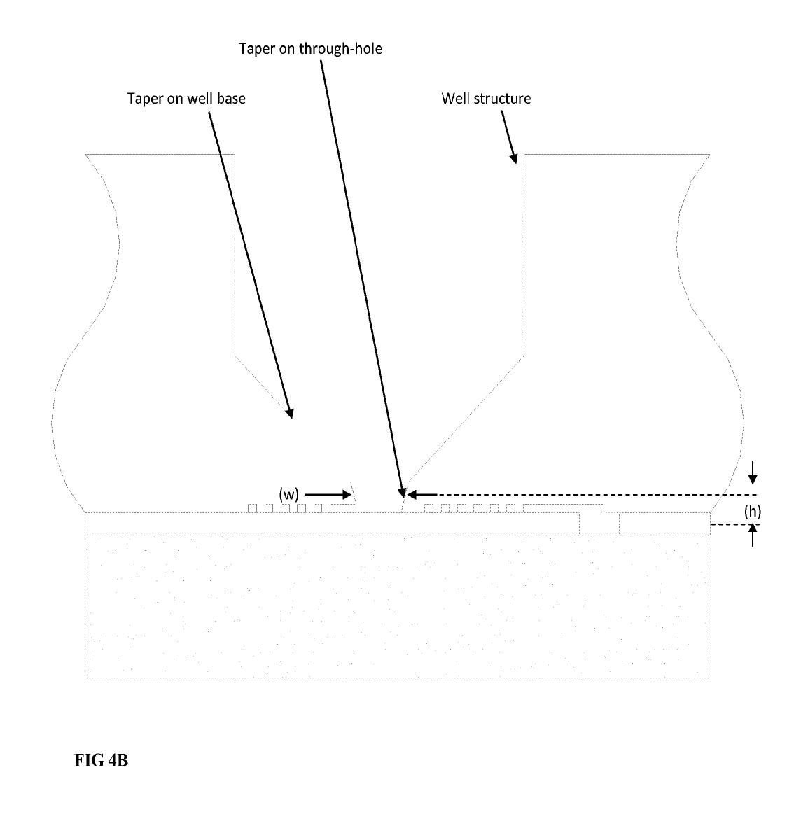

Another aspect of the present invention is shown in FIG. 4A. As shown in FIG. 4A preferably, the width of the hole (w) shall be greater than, and at least equal to, the depth (d) of the hole. This ensures that when liquid is introduced in the well, the front meniscus of the liquid can “dip” and touch the surface of the sealing tape. The meniscus also touches all 4 “walls” of the microchannel connected to one part of the hole (left hand side in above referenced figure).

Thereafter, capillary forces will draw the liquid from the well and fill the microchannel. In order to ensure that the liquid fills the microchannel at least one of the walls of the microchannel should be hydrophilic. In a preferred embodiment, the sealing layer is an appropriate adhesive film wherein the adhesive exhibits a hydrophilic behavior. This will ensure that when the liquid is loaded into the well and the front meniscus touches the sealing tape, the liquid will “spread” on the tape; touch the microchannel section and thereafter continue to be drawn into the channel. In alternate embodiments, the sealing layer may another plastic that is similar to the one used to fabricate the well and channel structures and the two are assembled using techniques well know in the art such as thermal bonding, adhesive film assisted bonding, laser or ultrasonic bonding to name a few. In the alternate embodiment; the channel may be “primed” by forcing a first liquid through the channel. This can be easily accomplished by positioning a pipette tip or other suitable liquid handling tool against the interface hole such that it creates a reasonable seal. Then injection of liquid will result in at least a part of the liquid being injected in the channel and thereafter capillary forces will ensure that the liquid continues to fill the channel. Extending this further, in a less preferred embodiment, not just the initial but all assay steps can also be easily performed by injecting solutions directly in the channels and wherein the well structure is only used a guide for the pipette or other fluid loading tool. In yet another embodiment, all the walls of the channel are treated to be hydrophilic by appropriate choice of surface treatments that are well known in the art. In yet another embodiment, the substrate material including all microchannel walls can be rendered hydrophilic using techniques well known in the art; and a hydrophobic sealing tape may be used. The choice of surface treatment (i.e. final surface tension of the walls with respect to liquids) depends on the intended assay application. In most cases, it is preferred to have a hydrophobic surface to allow for hydrophobic interaction based binding of biomolecules to the surface. In other cases, a hydrophilic surface may be more suitable for hydrophilic interactions of the biomolecule with the binding surface; and in even other cases; a combination of hydrophobic and hydrophilic surface may be desired to allow both types of biomolecules to bind.

In yet another embodiment of the invention, a first “priming” liquid is used to fill the channel. Liquids such as Isopropyl Alcohol exhibit an extremely low contact angle with most polymers and exhibit very good wicking flow. Such a liquid will fill the channel regardless of whether the channel walls are hydrophilic or hydrophilic. Once the liquid contacts the absorbent pad a continuous path is created to the loading well. Liquids added thereafter will be automatically drawn into the channel. In combination with the microchannel surface, the well surface may also be modified to enhance or detract from the capillary forces exerted on the liquid column. For example, if a strongly hydrophilic treatment is rendered on the well surface, the rear meniscus will have a strongly concave shape wherein the bulge of the meniscus is directed towards the bottom of the well. This meniscus shape will compete with the meniscus shape at the front end of the liquid column (before it touches absorbent pad) and ensure a slow fill. If on the other hand the well surface is rendered strongly hydrophobic the rear meniscus may achieve a convex shape wherein the bulge of the meniscus is towards the top of the well. This meniscus shape will add to the capillary force present at the front end of the liquid column and cause a faster flow rate.

The use of a liquid with low surface tension, such as but not limited to; isopropyl alcohol, isopropyl alcohol added at various concentrations to water, or an aqueous solutions with high protein concentrations may be particularly as a “priming” liquid in cases where the surface of the microchannel is modified by a protein adsorption step. For instance, frequently it is desirable to coat the first biomolecule in an assay sequence; namely the capture antibody, then block the surface and provide such a “coated” plate to the end-user. In this embodiment, the microchannel is coated with the desired biomolecules and then the channel is dried out (i.e. the liquid is allowed to evaporate completely). This minimizes the number of steps that the final end-user has to complete to achieve the desired immunoassay result. However, it is likely that the adsorption of the capture antibody and materials within the blocking buffer may render the surface of the Optimiser™ microchannel to a less hydrophilic state which may impede flow of the first reagent added by the end-user. The use of a low surface tension liquid such as ones outlined above will allow the first “priming” liquid to be drawn into the microchannel via capillary action even with the reduced hydrophilic effect of the microchannel. Thereafter, the priming liquid will create a liquid column extending from the inlet at the base of the loading well to the end of the microchannel and subsequently liquids will flow effectively using mechanisms described above. In a preferred method of the invention, an aqueous buffer solution is used as the “priming” liquid. During experiments, we have observed that priming with a common buffer solution such as Phosphate Buffer Solution (PBS) or Tris Buffer Solution (TBS) leads to enhanced binding of the first biomolecule introduced thereafter. This is a noticeably different behavior as compared to the conventional microplates even though the two platforms share the same polymer substrate material. We hypothesize that the priming liquid may increase the wettability of the surface thereby allowing the second solution, containing the first biomolecule introduced in the microfluidic microplate, a more uniform contact with the surface thereby leading to higher binding of the biomolecule to the polymer surface. The aqueous priming solution can either be injected (using applied pressure or vacuum) into the microchannel, or alternately by using a microchannel embodiment wherein at least one wall exhibit hydrophilic behavior allowing for capillary fill of the priming solution. Furthermore, the effect of the aqueous priming liquid is different for each assay. As described further in the application, certain assays show significantly improved performance with the PBS buffer prime, whereas other assays do not exhibit a significant improvement. The latter assay types are distinguished from the former in that the latter assay types already exhibit a strong response on the Optimiser™—hence by using the priming step as a consistent guideline for all assays the performance may improve but will certainly not deteriorate.

A noticeable difference is observed in the effect of pH of at least one material used for ELISA assays on the microplate utilized in the practice of the present invention; namely, the pH of the coating buffer. In conventional microplates, the effect of pH on binding of capture antibody to the polymer well surface is well documented and well known in the art. However, in conventional microplates, the pH effect is obvious only with large variations in pH. Most commonly pH of the coat buffer is either of approximately pH 7 or approximately pH 9.3 or approximately pH 2.8. Most assays on conventional microplates that work well with one of the pH values listed above, also work moderately with other pH values. However, the Optimiser™ is exquisitely sensitive to pH variations of the coat buffer. As described in detail in Case Study 3 of this disclosure, the microplate shows a pronounced change in assay signal (10×) which is significantly different from the conventional plate assay behavior. Furthermore, the optimal pH for an assay is not a constant for the assays of this invention, and different assays require the use of different coat buffers to achieve best performance. Even further, the range of pH variation across the ideal pH value for a given assay is also dependent on the type of assay. This is an unexpected finding and establishes that selection of optimal coat buffer pH is of particular significance for the microplate based assays contemplated by this invention.

In other preferred embodiments of the present invention, the sealing layer can be configured to be reversibly attached to the microchannel substrate. In this configuration, the sealing layer can be removed for a portion of the fluidic steps; for example for absorbance assays; the sealing layer can be removed gently and a stop solution is added to stop the absorbance reaction. In even other embodiments, the sealing layer may be a specific material that is suitable for other methods of assay analysis; for example the sealing layer may be chosen to be particularly well suited to capture immuno-precipitation by products from a relevant assay.

In another embodiment shown in FIG. 4B, the through-hole structure itself may be tapered rather than a cylindrical geometry with straight sidewalls as shown in FIG. 4A. The taper shape will assist in the capillary action in drawing the liquid from the well via the through hole to at least one hydrophilic microchannel wall. In yet other embodiments; the well and through-hole structures shown in FIG. 4A or FIG. 4B may be selectively treated to impart a different surface functionality. For instance, the substrate layer may be substantially hydrophobic with only the inside surface of well and the through-hole treated to be hydrophilic. The substrate layer is turn sealed by a hydrophilic tape. Hence in this configuration; there is a continuous hydrophilic path from the well to the through hole to the base of the microchannel (tape) ensuring that the liquid consistently fills the microchannel without any intervening air bubbles.

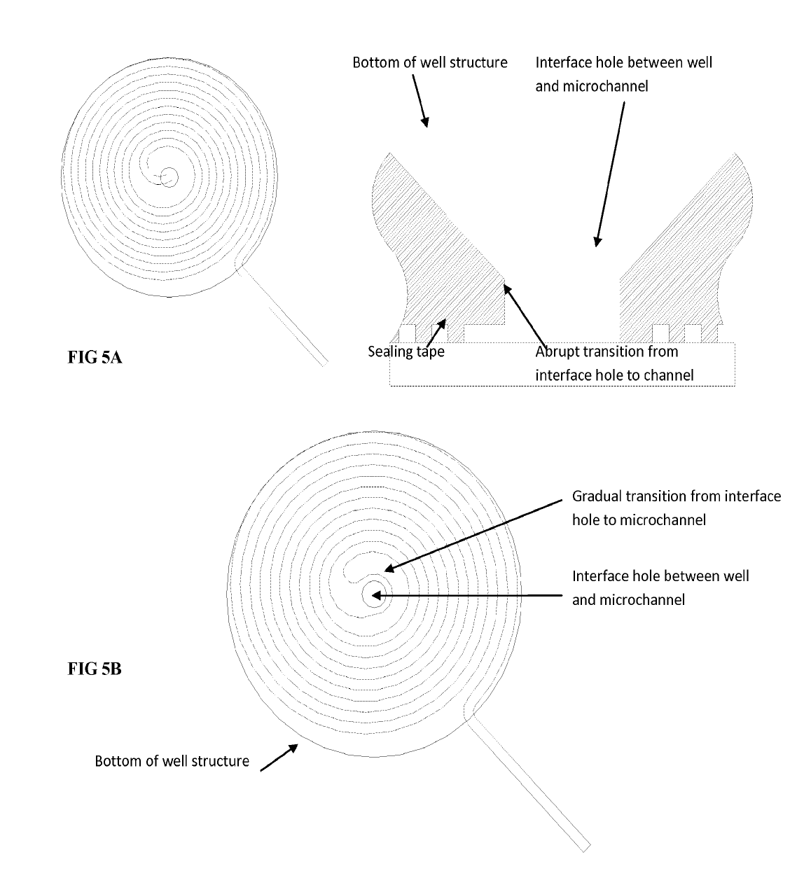

Another aspect of the present invention is shown in FIG. 5. FIG. 5 shows embodiments of the microchannel configuration at the interface hole between the well and the microchannel. In FIG. 5A; there is an abrupt transition from the cross sectional area of the through hole to the cross sectional area of the microchannel. Since the cross sectional area of the channel is much smaller; the liquid exiting the well will stop at the interface. In FIG. 5B the microchannel is slightly larger than the interface hole and furthermore, the channel cross section gradually tapers to the final dimension. In this case, as the liquid exits the well, it will continue to flow (into absorbent pad) until even the microchannel is completely emptied. Alternately an absorbent pad with very high capillary force can be used such that even with the configuration of FIG. 5A the microchannel is completely emptied. In the former case, wherein the liquid remains in the microchannel until the next liquid is added, the condition can be used as an incubation step. It is advantageous to use this configuration since in this case, the assay performance is relatively independent of slight variations in flow rate that may occur if a purely flow through assay is used. The latter case, wherein the liquid never stops in the channel; alternatively called a continuous-flow or through flow assay; the assay operation is significantly quicker. This may be advantageous in applications wherein in response time is more critical than control over precision as is the case for some point-of-care test applications. The flow-through mode may also be exploited advantageously to increase the sensitivity of detection. For instance, when the first binding agent (capture antibody) is already coated on the microchannel walls; and remaining unbound binding sites are blocked; a much larger volume of sample (containing target antigen or analyte) can be loaded in the well. As the liquid slowly flows past the channel wall; an increasing amount of antigen can link with the capture antibody on the surface. In effect, the flow-through mode serves to replenish the supply of target antigen/analyte exposed to the binding sites until a large fraction of the binding sites are linked with the antigen. Then a detection or secondary antibody is linked to the bound target as described earlier and this scheme can detect much lower concentrations of the target from a given sample. The rapid reaction kinetics on the microscale ensures that a significant portion of antigen can link with the capture antibody within the short duration that the liquid is within the channel in flow through mode (few seconds).

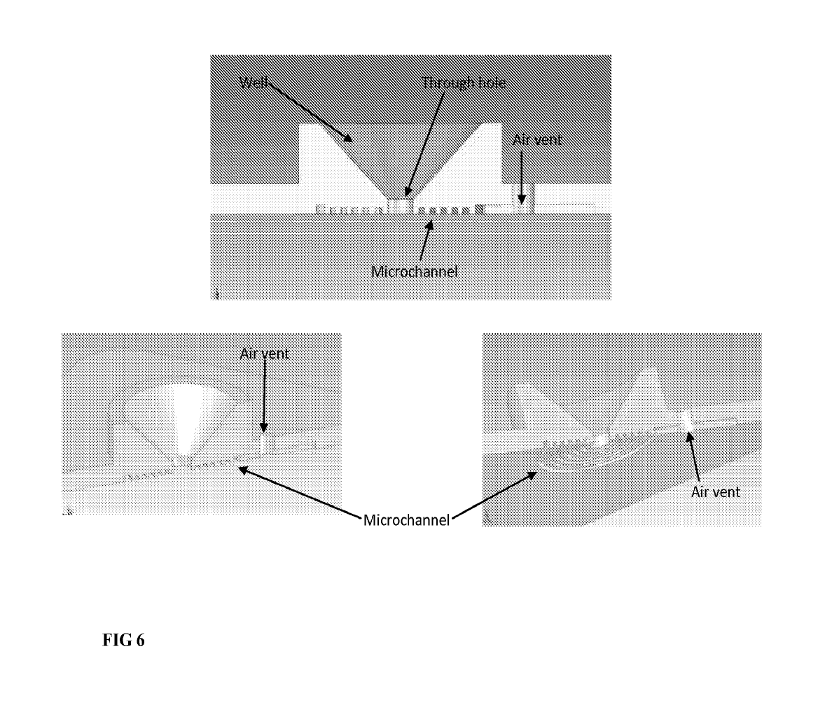

FIG. 6 shows a feature of the invention that further aids in the reliable performance of the flow sequence wherein an incubation step is desired. As shown in FIG. 6, an air-vent hole is configured towards the outlet of the microchannel in close proximity to the outlet hole on the tape. With this configuration, as the liquid is emptied from the well, the rear end of the liquid will get “stuck” at the interface between the through-hole and the microchannel. A high capillary force absorption pad may continue to exert a capillary force that would normally cause the liquid to empty from the microchannel as well. In effect, the absorbent pad is acting as a vacuum source and creating a negative pressure at the front end of the liquid column. As the liquid is sucked out by the absorbent pad, the liquid column will “retract” back into the microchannel. When the front end of the liquid channel retract beyond the air-vent hole, the capillary action of the pad will come to a halt, since the negative pressure (from the pad) is relieved by atmospheric pressure via the air-vent hole. The air vent hole can also be positioned inside the perimeter of the outlet hole on the sealing tape. The latter configuration will ensure that as soon as the liquid retracts slightly (due to continued absorption by pad), the air-vent will allow the negative pressure to dissipate. As described further, it is necessary to ensure that the liquid retracts backwards, i.e. away from the outlet. If the liquid front were to remain stationary (at outlet) and instead if the rear end of the liquid column (at the through-hole interface) were to move into the channel; i.e. away from inlet; an air-bubble would be formed when an additional liquid is loaded in the well. The intervening air-bubble between the two different liquids would cause the capillary action to stop and prevent further operation.

An important aspect of the current invention is the use of microfluidic channels to perform the immunoassay as opposed to the well structure in a conventional microplate. It is well known in the art that the high surface area to volume ratio of the microchannels allows for (a) rapid reactions due to limited diffusion distances and (b) low reaction volumes. A wide variety of microchannel configurations may be used for this invention. As shown in the TABLE below, the surface area to volume ratio increases as the channel size decreases, with an attendant decrease in liquid volume required to completely fill the channels. The channel dimension will be determined based on requirement for flow rate, surface area, and surface area to volume (SAV) ratio. For example; assuming a 500 um loading well in the center, and wherein the radius of the largest spiral channel is approximately 3 mm; the following configurations are possible. All such variations are intended to be and should be considered within the scope of this invention.

TABLE 1
Effect of channel dimensions on surface area to volume (SAV) ratio
Effect of channel dimensions (approximate)
Assuming width (w) = depth (d) = spacing (s) of spiral channel
Increase in Area is with reference to bottom area of a 96 well plate

| | | | Inc in | Vol. | SA/V |
|---|---|---|---|---|---|
| w, d, s | Length | Area | A | (μl) | ratio |
| 0.05 | 152 | 30.44 | 8% | 0.38 | 80.10526 |
| 0.1 | 109 | 43.73 | 55% | 1.09 | 40.11927 |
| 0.2 | 84 | 66.85 | 136% | 3.34 | 20.01497 |
| 0.5 | 75.4 | 150.8 | 433% | 18.85 | 8 |

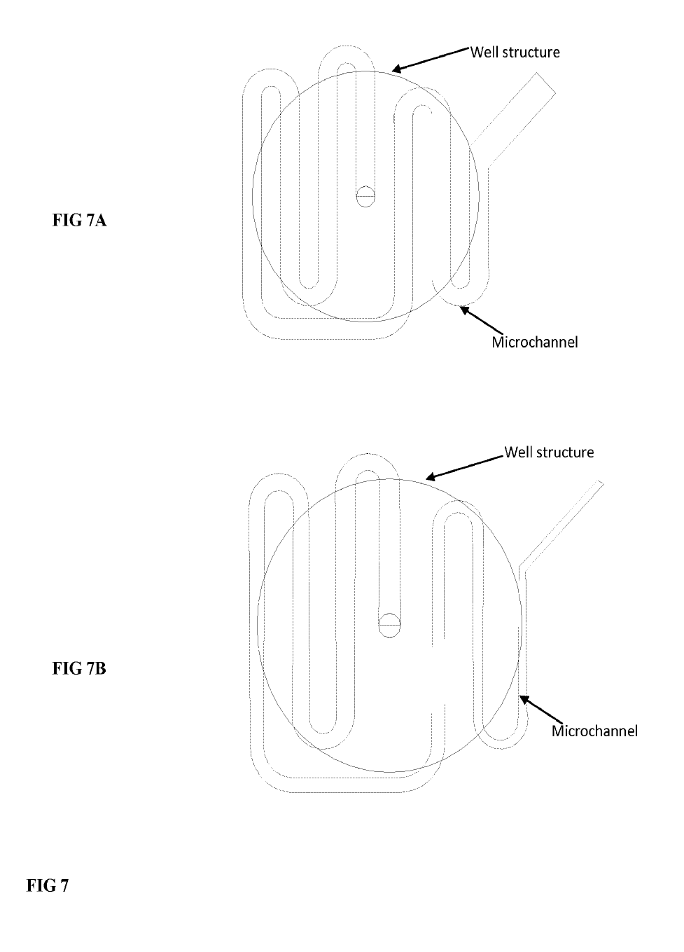

Of course, a wide variety of channel configurations are also possible in addition to the spiral shown in earlier figures. FIG. 7A shows a serpentine channel which is equally well suited to the present invention. Furthermore, the channel may include a continuous taper from the inlet to the outlet. The taper will ensure that there is increasing capillary force on the front end of the liquid column and result in a different flow rate than in the case when the channel is not tapered. In other embodiments, the taper may be designed from the outlet to the inlet such that the channel gradually widens from inlet to outlet. This will result in yet another flow rate compared to the first taper or when there is no taper. The difference in flow rate may have a significant impact on continuous-flow through flow assays or the liquid filling behavior for static incubation assays and can be advantageously used to afford further flexibility. In yet other embodiments, the channels may be configured to be non-symmetric i.e. width not equal to depth not equal to spacing or combinations thereof.

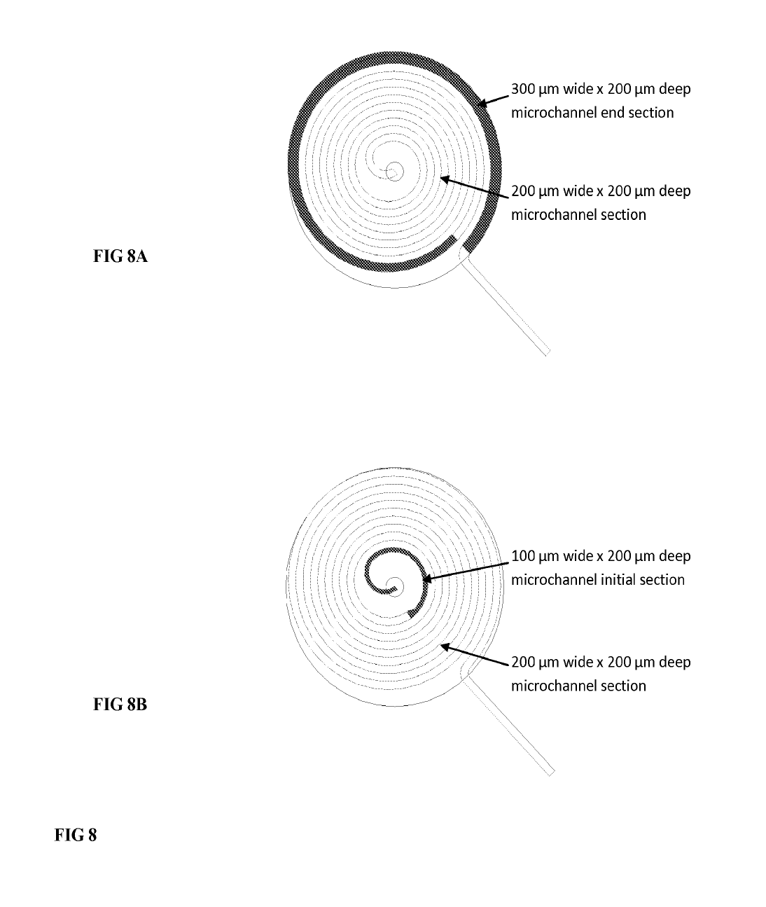

Other embodiments for the microchannel are illustrated in FIG. 8. As shown in FIG. 8A; the microchannel has a composite geometry wherein the microchannel cross-sectional dimensions at the highlighted end section are different compared to the cross-sectional dimensions of the rest of the microchannel. The end microchannel section has at least one dimension larger than the comparable dimension for the rest of the microchannel. For example, the end section may be 300 μm wide×200 μm deep whereas the rest of the microchannel may be 200 μm wide×200 μm deep. This ensures that the end section has a lower flow resistance than the preceding channel. This is useful in ensuring optimum flow performance for the static incubation case. As described earlier in conjunction with the explanation for FIG. 6, it is preferred that continued action of the absorbent pad draw liquid out such that the liquid retracts backwards from the outlet. The embodiment shown in FIG. 8 can ensure that the since the flow resistance for the front end of the liquid column (closer to outlet) is lower than the flow resistance for the rear-end of the liquid column (at through-hole interface), the liquid will always “retract” backward from the outlet.

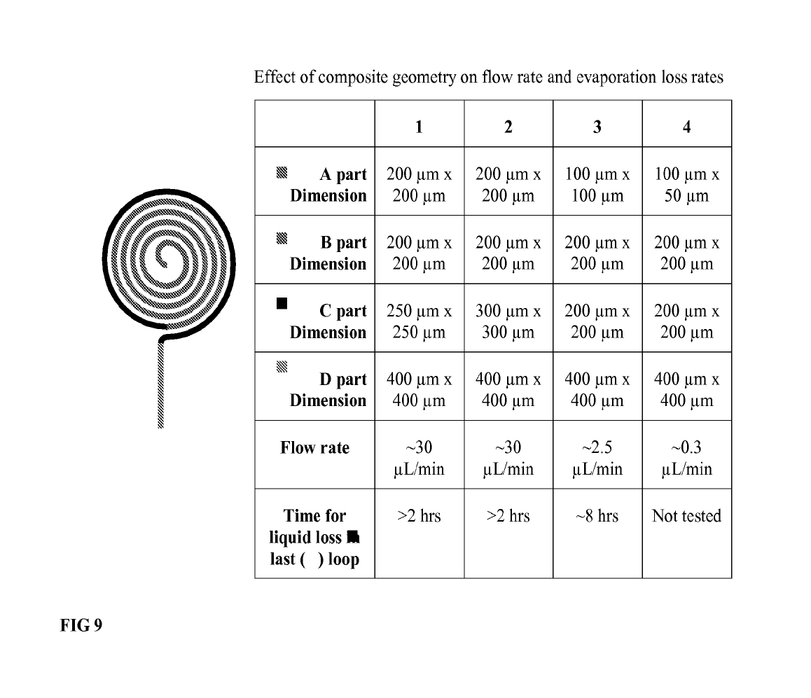

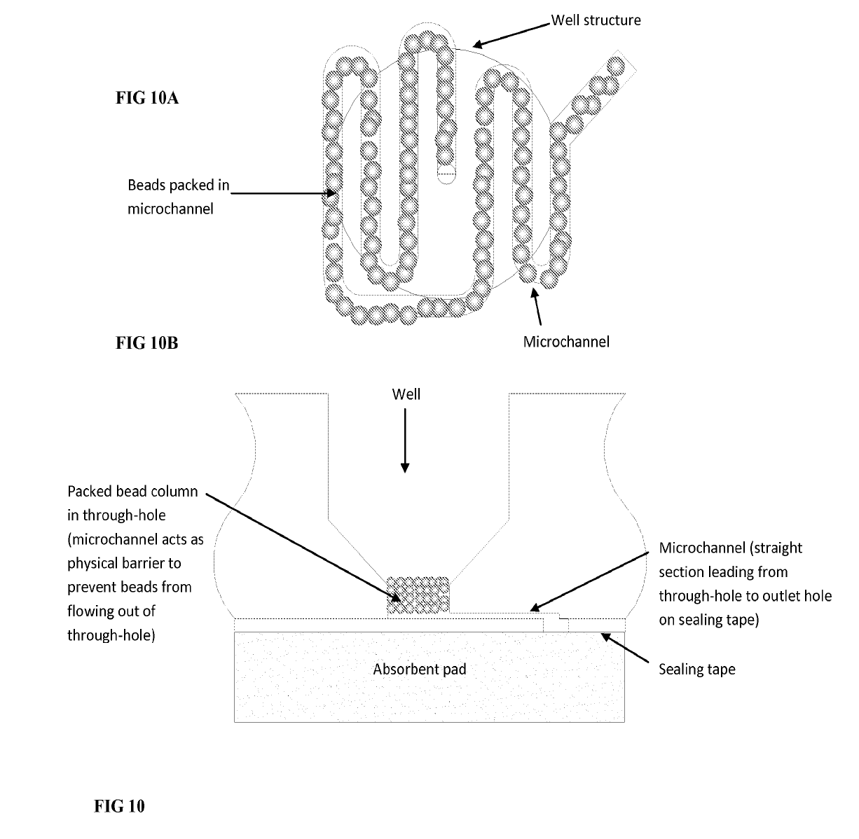

Another embodiment that can achieve is a similar effect is shown in FIG. 8B; wherein the highlighted initial section is different compared to the cross-sectional dimensions of the rest of the microchannel. The initial microchannel section has at least one dimension smaller than the comparable dimension for the rest of the microchannel. For example, the initial section may be 100 μm wide×200 μm deep whereas the rest of the microchannel may be 200 μm wide×200 μm deep. This ensures that the initial section has a higher flow resistance than the remainder. This will also ensure that the liquid always retract backward; i.e. away from the outlet rather than retracting into the channel; i.e. away from the inlet. Furthermore, the use of a high resistance section at the start of the microchannel is also advantageous for flow regulation for continuous-flow or flow-through mode. As shown in FIG. 9 and the associated TABLE, the flow rate within the microchannel is highly dependent on the microchannel dimension. The flow-through mode requires (1) a precise control over the flow rate to ensure repeatable performance and (2) ability to flow at low flow rates to allow for sufficient residence time for liquid flow through the channel to ensure maximum adsorption/linking of biochemicals in liquid to the ligands on the channel walls. As illustrated in the different dimensions shown in FIG. 9 and associated TABLE, a combination of these embodiments may also be used for added flexibility.

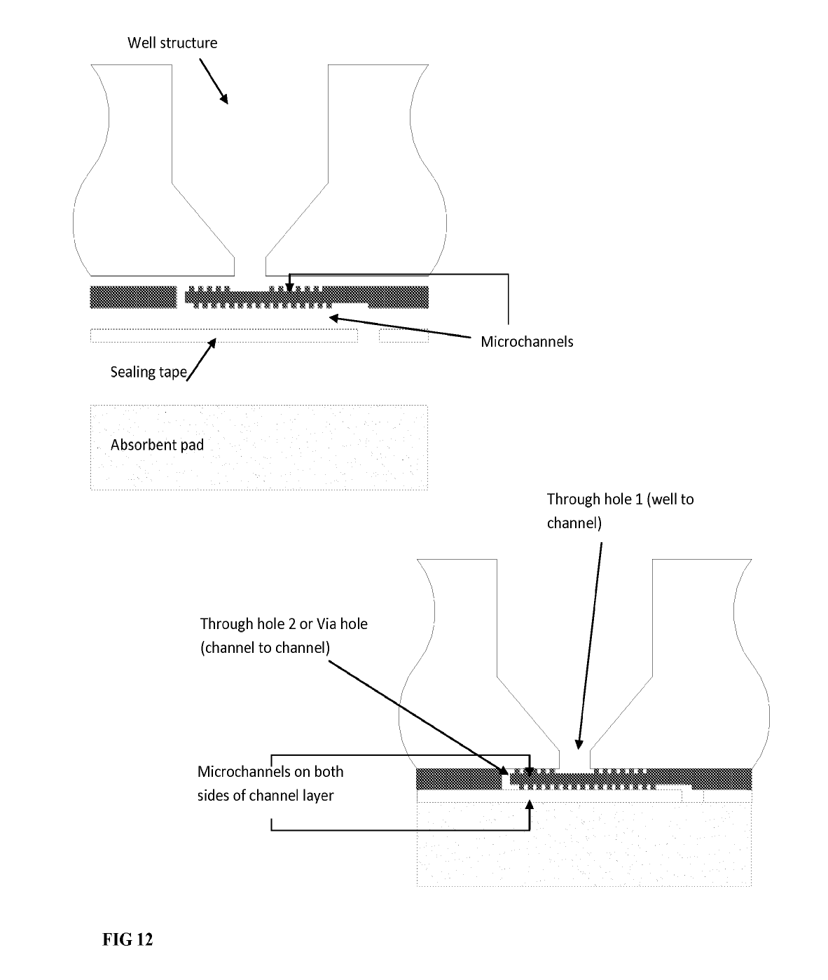

An alternate embodiment is shown in FIG. 12, wherein the well structure and the microchannel structure are defined on two different substrates. In this embodiment, the microchannel is defined on two faces of the substrate such that channel on one face correspond to wall regions of the second face and vice versa. This ensures that there is no wasted space in the horizontal footprint of the well bottom and a greater assay signal can be generated.

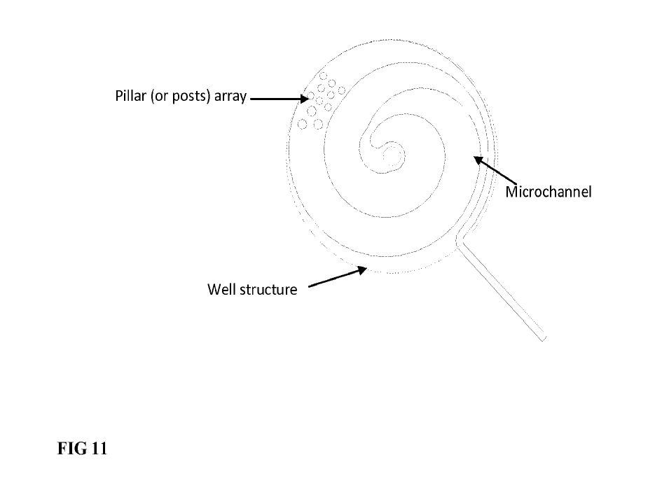

As explained earlier; the advantage of microchannels over conventional scale analysis chambers is the high surface area to volume ratio within channels. This can be further magnified by the use of a variety of techniques well known in the art. One such approach is shown in FIG. 10A; wherein the channel is packed with an array of beads. A wide variety of beads can be used for this application including magnetic, non-magnetic; polymer, silica; glass beads to name a few. Alternately, the channel can have monolithic polymer columns created using self-assembly or other appropriate assembly methods. All of these, and other well known techniques in the art, can significantly increase the net surface area inside the microchannel and can allow for even faster reaction times than microchannel devices. The use of beads allows for greater flexibility in device operation as further explained later in this description. When beads (polymer or otherwise) are to be used—they are directly dispensed onto suitable sized hole at bottom of well. The channel dimension is selected such that beads can flow freely through them. Then the beads will flow all the way to the outlet till they reach the absorbent pad which will prevent further motion of beads. At this stage, the absorbent pad may be replaced if desired to remove any residue of solution in which beads are suspended. Further steps will remain the same. Alternately, the beads may be packed by using self assembly techniques or slurry packing methods.

In a particularly preferred embodiment, the beads are the Ultralink Biosupport™ agarose gel beads. These beads offer a porous surface area that greatly magnifies the surface area of the beads. Furthermore, the beads are well suited for covalent linking of biochemicals such as capture antibodies. After a high surface concentration of the capture antibody is linked to the beads, the remainder of the bead surface can be effectively passivated to minimize non-specific adsorption. The Ultralink Biosupport™ beads are commonly used in affinity liquid column chromatography such as Fast Protein Liquid Chromatography (FPLC) and their use in microfluidic channels allows for a tremendous increase in sensitivity. For FPLC applications, the beads are “prepared” by covalent linkage of capture entity and subsequent passivation in liquid containers such as test tubes, and then packing beads in the FPLC column. For the microfluidic microplate, a similar approach can be used, and alternately these processes can also be performed by first entrapping the beads in a suitably configured geometry and then adding the linking chemistry and passivation solutions in series. This offers greater flexibility in providing “generic” microplates pre-packed with beads and allowing the end-user to link the desired chemistries to the beads.

The embodiment shown in FIG. 10A is particularly well suited for applications where extremely high sensitivity is desired. An alternate embodiment using microbeads is shown in FIG. 10B. As shown in FIG. 10B, the beads are only trapped in the through hole connecting the well to the channel. In fact the channel dimensions are configured such that the channel acts as trapping geometry and the narrow dimensions do not allow any beads to enter the channel. It is important to note that in this embodiment, the small bead packed column is the “reaction chamber”, and the microfluidic channel only serves to transport the liquid away from the base of this bead column to the outlet and is consequently only a straight section. The extremely high binding capacity of the Ultralink Biosupport™ beads allows for adequate sensitivity in immunoassay applications even when a very small “bead column” as illustrated in FIG. 10B is used. This embodiment is particularly well suited for high density microplates such as the 384-well and 1536-well configurations.

As described above, one technique to use the beads (such as the commercially available Ultraink Biosupport™ or others) is to coat the beads with the desired agent and then load them into the channel (or through hole). This approach limits the microplate to the antigen that will react with the coated capture molecule. At the same time, the “pre-coating” also renders the bead surface hydrophilic allowing for capillary flow to occur within the bead packed column. For the “generic” microplate wherein uncoated beads are used, the hydrophobic surface of the uncoated/non-passivated beads will greatly reduce if not completely inhibit capillary flow. In order to circumvent this problem, a mixture of treated and untreated beads can be used. For example, when the beads are prepared for loading (in the manufacturing facility) an appropriate ratio of untreated (hydrophobic) and passivated (surface rendered hydrophilic) can be mixed and loaded in the channel or through hole. This will ensure that the packed bead column can support capillary flow action at the expense of reduced binding sites (on passivated beads). Despite the reduction, the net number of binding sites will still be considerably higher than the binding sites only on the walls of the microchannel.

It is to be appreciated that the present invention is not limited to assay analysis only. For example, the embodiment shown in FIG. 11 may be used for cell based analysis. The pillar array within the channel can entrap cells as they are transported from the well and trapped at precisely defined locations. Thereafter, the cells may be exposed to different chemical to study the effects of such chemicals on certain cellular functions. In certain cases, the response may be in form of chemical released from the cell. In this case, the assay sequence can be configured such that after the cell solution is added and before the stimulating chemical is added, the absorbent pad(s) is replaced with a new pad. Hence the chemicals released from the cells can be collected into the absorbent pads and further analyzed. In other embodiments, the surface of the microchannels may be suitably treated to ensure that cells can adhere to the walls. In this example, the cells can first be cultivated and grown in the microchannels and subsequently exposed to test chemicals.

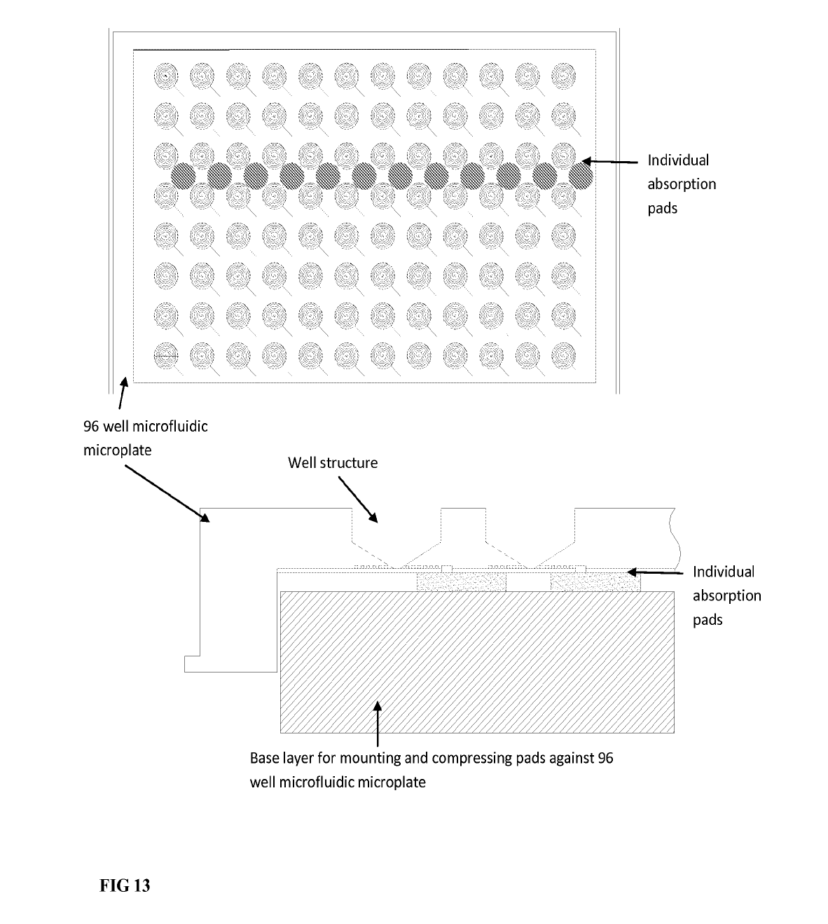

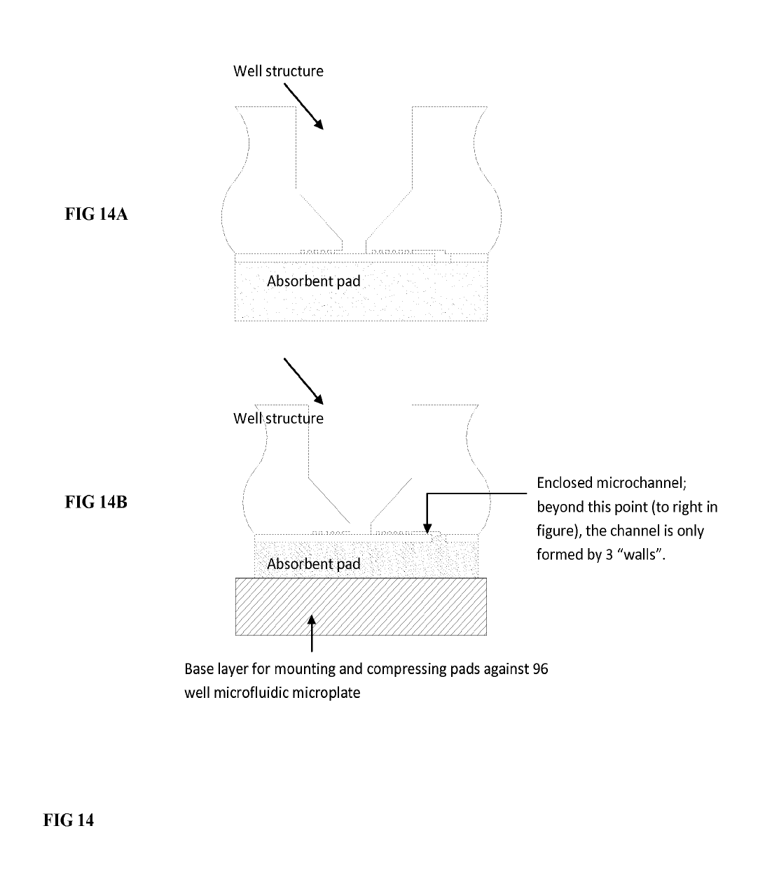

In all embodiments of this invention, the absorbent pad may be common for all fluid handling steps or may be configured such that it is replaced after each fluid handling step or after a selected set of steps. Furthermore, the absorbent pad may be removed after the final fluid processing step or may remain embedded in the microfluidic microplate. In the preferred embodiments, the absorbent pads are configured such that they do not overlap the microchannel and/or well structures. This ensures that there is an optically clear path for detection of assay signal without removing the absorbent pads. FIG. 13 shows one such embodiment, wherein a unique absorbent pad is used with each well+channel structure. Also as shown in FIG. 13; the absorbent pad may be located on the microplate or may be located on a separate layer. In the latter case, the microfluidic microplate is positioned over the substrate holding the absorbent pads using an appropriate jig. Naturally, in all cases the absorbent pad may also be a continuous sheet common to all the “wells” of the microfluidic microplate.

A potential problem with using continuous absorbent pads in a completely transparent configuration is the fact that the pad will soak up all assay reagents (including the optically active components). It is then impossible to distinguish the optical signal from the microchannel from the optical signal from the absorbed components in the pad. In most embodiments, the sealing tape is envisioned as a hydrophilic adhesive on a transparent liner. In cases wherein the absorbent pad is a continuous sheet, the sealing tape can be selected such that the hydrophilic adhesive is deposited on an opaque liner. The tape is punch-cut to create an outlet hole similar to the one previously described. The end of the microchannel and the outlet hole is positioned away from the vertical viewing window of the well and the spiral microchannel pattern. This embodiment with the opaque tape liner will allow for a continuous sheet of the absorbent pad to be used without the optical cross-talk effect since the only “window” to the pad will be the punch-cut hole on the sealing film which in turn is positioned away from the viewing window. The microfluidic microplate is limited to a “top-read” mode; but the pad can be integrated as part of the microplate thereby eliminating the need for a holder. The embodiments will partly be dictated by application; for example: for manual use, a removable pad is easy for an operator to remove prior to reading whereas for High Throughput Screening using automated equipment it is preferred to have the pad integrated for compatibility with current instruments.

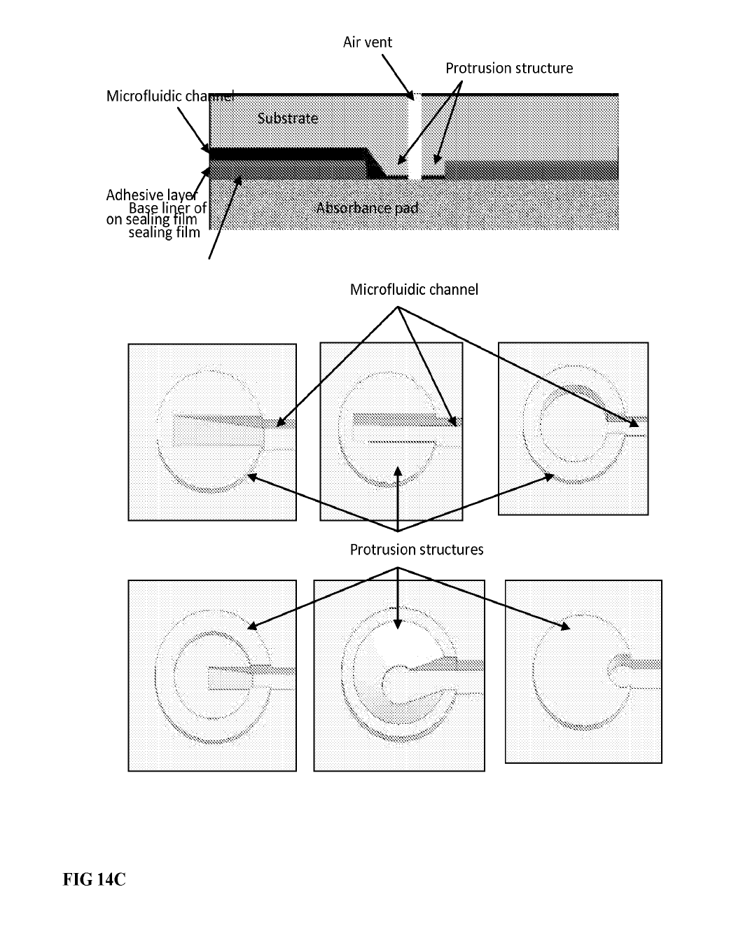

As shown in FIG. 5, the abrupt transition from the through hole at the bottom of the well and the microchannel leads to an abrupt change in surface tension pressure of the liquid column and stops flow at that interface. A similar situation may also occur at the outlet end as shown in FIG. 14A. The use of an additional base layer to compress the absorbent pad can ensure that the relatively flexible absorbent pad will bulge into the cavity created on the sealing film; as shown in FIG. 14B. The bulge will furthermore directly touch the microchannel cross-section where the microchannel interfaces with the outlet hole. This can ensure that the absorbent pad is always in “contact” with the exiting liquid. Alternately as shown in FIG. 14C a protrusion structure may be fabricated at the end of the microchannel in the outlet section. The protrusion structure may be configured such that the flat surface of the protrusion structure (away from substrate) approximately aligns with the surface of the sealing tape (away from substrate); thereby minimizing the transition effect. FIG. 14C shows a range of geometries that can be used to create the protrusion structure.

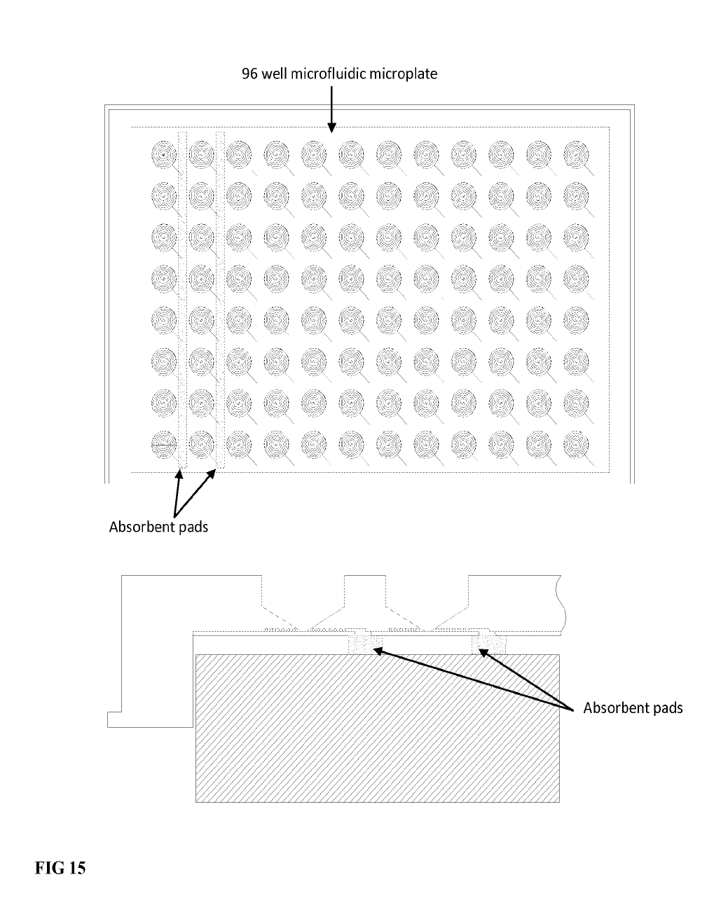

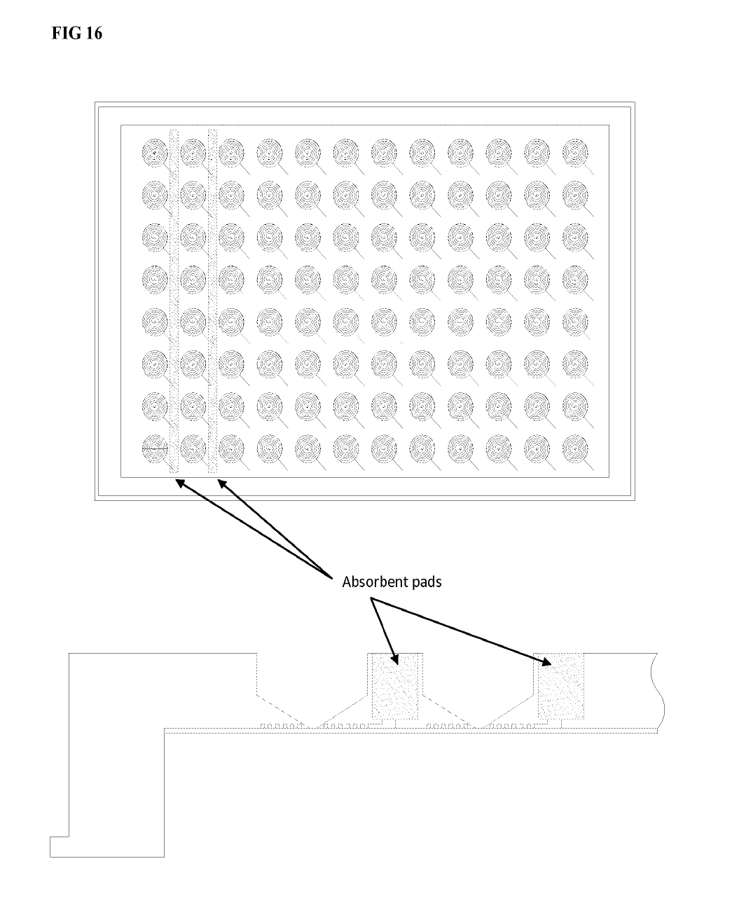

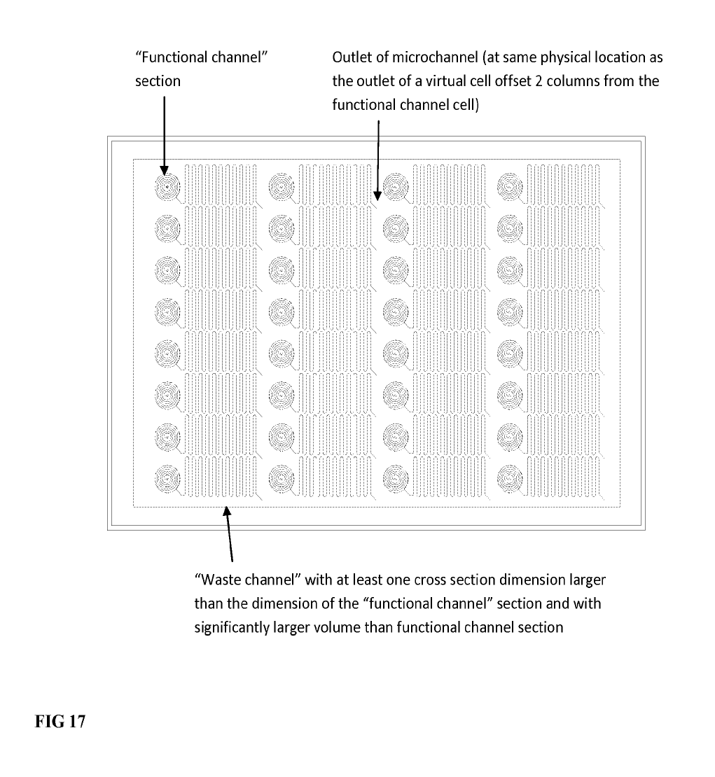

FIG. 15 shows another embodiment wherein the pads are configured as strips furthermore where one strip of absorbent pad is common to a row (or column) of well+channel structures. FIG. 16 shows even yet another embodiment wherein the absorbent pad strips are positioned from the “top”; i.e. on the opposing face from the microchannels. Thus, a wide variety of configurations can be used to position the absorbent pads without departing from the spirit of the invention.

As is also readily evident to those skilled in the art, any material that is capable of exerting a capillary force higher than that exerted by the microchannels is suitable for use as absorbent pad, in the devices of the invention. A wide variety of materials such as filter papers, cleanroom tissues etc. are readily obvious examples. Other esoteric absorbent “pads” may include a dense arrangement for example of micron sized silica beads in a well structure. These would exert extremely high capillary force and all are envisioned as absorbent pads within the present invention.

In fact, a preferred embodiment wherein the microchannel itself is used as capillary pump and waste reservoir is illustrated in FIG. 17. As shown in FIG. 17, the architecture is modified such that fewer wells are “functional” on the 96-well layout. Each well is connected via through-hole to a microchannel. The microchannel in this embodiment is divided in two zones; the “functional” channel and the “waste” channel. The waste channel is designed such that it can accommodate all the liquid that is added during a multi-step assay sequence. As the first liquid is added it will flow through the initial “functional” sectional of the channel wherein the assay reactions as described previously would occur on channel walls. Thereafter the first liquid will reach the “waste” section of the continuous microchannel. The hydrophilic tape will continue to exert a capillary force and draw the liquid out of the well. Using a larger cross-sectional area in the “waste” section of the channel, ensures that the capillary force at the “waste” channel is weaker than the capillary force at the through-hole: microchannel interface thereby stopping flow when the first liquid is drained out of the well. As the second liquid is added to the well, the capillary barrier at the base of the through hole is eliminated and flow will resume till the second liquid is drawn out of the well. This embodiment allows for a fully-integrated device without the need for an absorbent pad. Furthermore, in this embodiment the air-vent is also not required since the flow is automatically regulated by the difference in dimensions between the “functional” and the “waste” channel sections. This embodiment may allow for greater reliability by minimizing the number of components used. In yet other embodiments, the waste channel may only be a through hole (directed “upwards”) extending through the substrate layer forming the microplate. A reasonably thick substrate layer; which may further be non-uniform in thickness, will allow for sufficient liquid to be contained in a “waste well”. The alternate embodiment can allow for use of the microfluidic capillary pump concept without sacrificing well count.

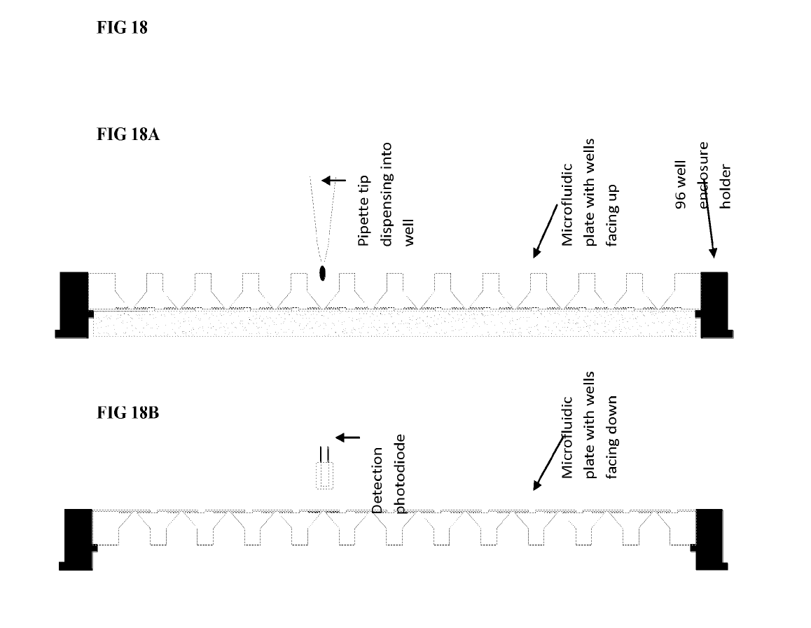

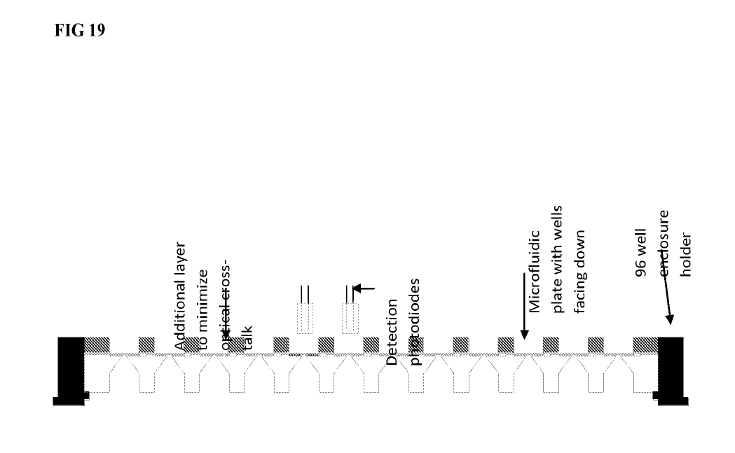

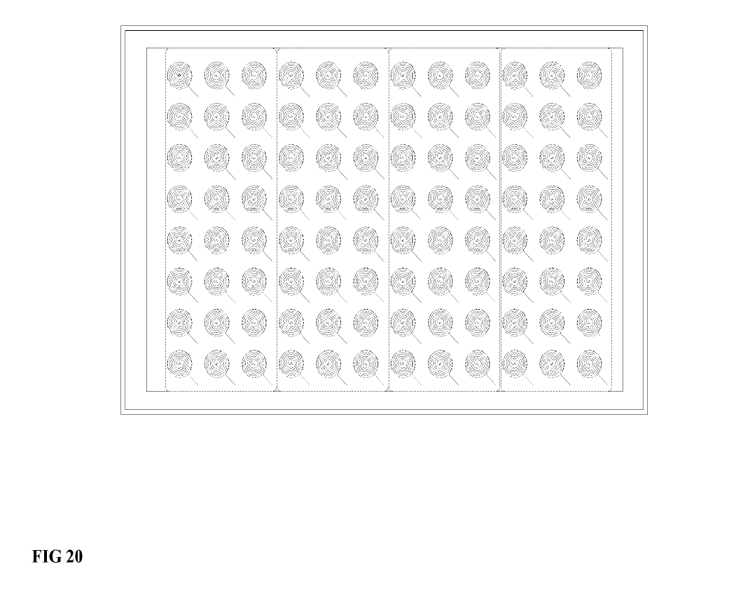

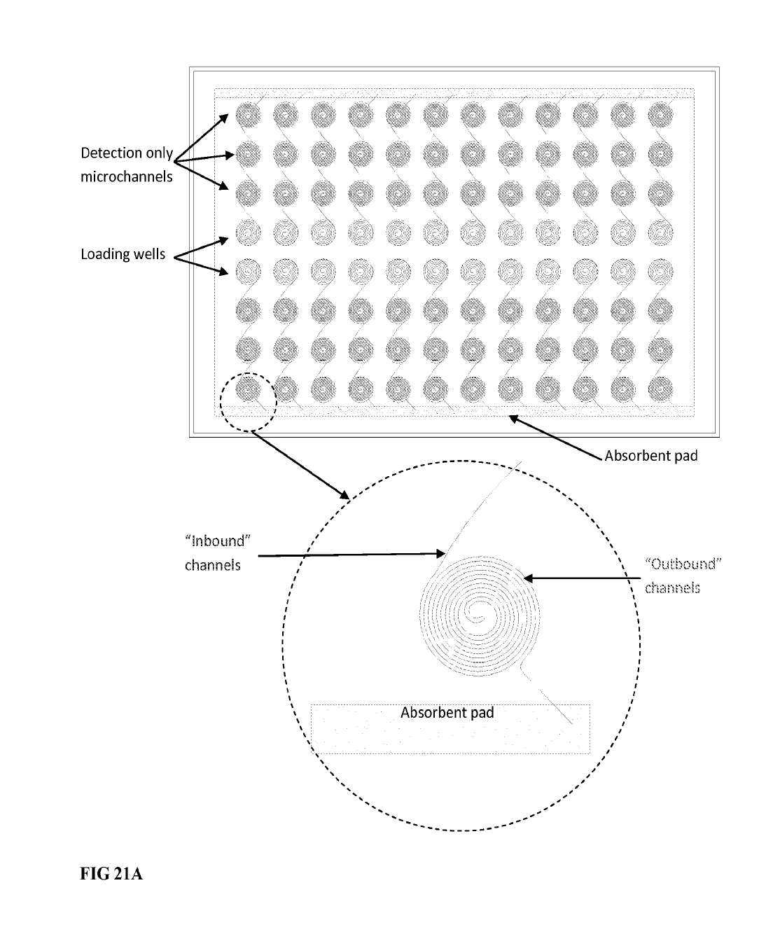

Hitherto, the microfluidic channels and the wells are described as being a part of the same structure that also defines the external shape to match the footprint of a 96 well plate (with the exception of the embodiment shown in FIG. 12 wherein only the wells are part of the “microplate” substrate). It may actually be more advantageous to use the embodiment shown in FIG. 18. As shown in FIG. 18 a microfluidic insert plate is used with a surrounding enclosure—wherein the enclosure defines the shape and footprint (along perimeter) of a conventional microplate and wherein the microplate insert structure contains the well structures and the microchannel structures. The two parts may be designed such that the microfluidic insert plate can be removed from the enclosure. The use of this is illustrated in FIG. 18; wherein in one orientation; specifically where the wells are facing the top; the device is used for the assay fluidic sequence and in another orientation; specifically when the microchannel part of the microfluidic insert plate is facing up; the device is used for assay detection sequence. The enclosure may be designed such that the microfluidic insert plate can be positioned at a height that is optimum to ensure best signal from the microchannel by ensuring that the microchannels are located in the same focal plane as that of the photodetectors. This embodiment is especially well suited for fluorescence detection wherein a directional beam of light is used to cause fluorescence. For chemiluminescence applications an embodiment shown in FIG. 19 may be more suitable. In this embodiment, an additional plate is positioned on top of the inverted microfluidic insert plate. The additional plate contains openings in the regions of the microfluidic insert plate wherein the microchannels are positioned whereas the walls of the structures forming these openings are opaque. This can ensure that there is considerable reduction in the “optical cross-talk” effect where signal from one reaction chamber reaches multiple photodetectors. The embodiment of FIG. 18 is also suitable for use with an opaque substrate such that after rotation, the channel side can be read by a “top” reading microplate reader. In another alternate embodiment, the device of FIG. 12 may be fabricated such that the “well’ part of the device is made from an opaque material whereas the “channel” part is made on a transparent substrate. An alternate embodiment is also shown in FIG. 20 wherein multiple microfluidic insert plates are used. The array of inserts may be configured for a particular size such as a standard glass slide footprint of ˜25 mm×˜75 mm to allow; for example liquid handling equipment designed for microplates to manipulate 4 inserts simultaneously, and a slide reader to read each of the microfluidic inserts separately; in a mix-and-match manner.

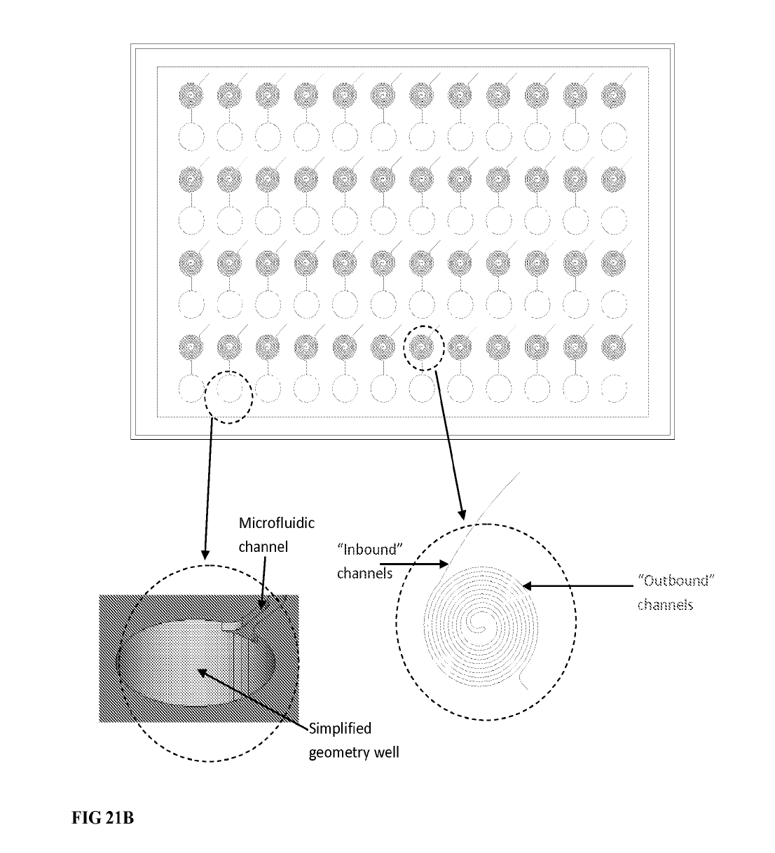

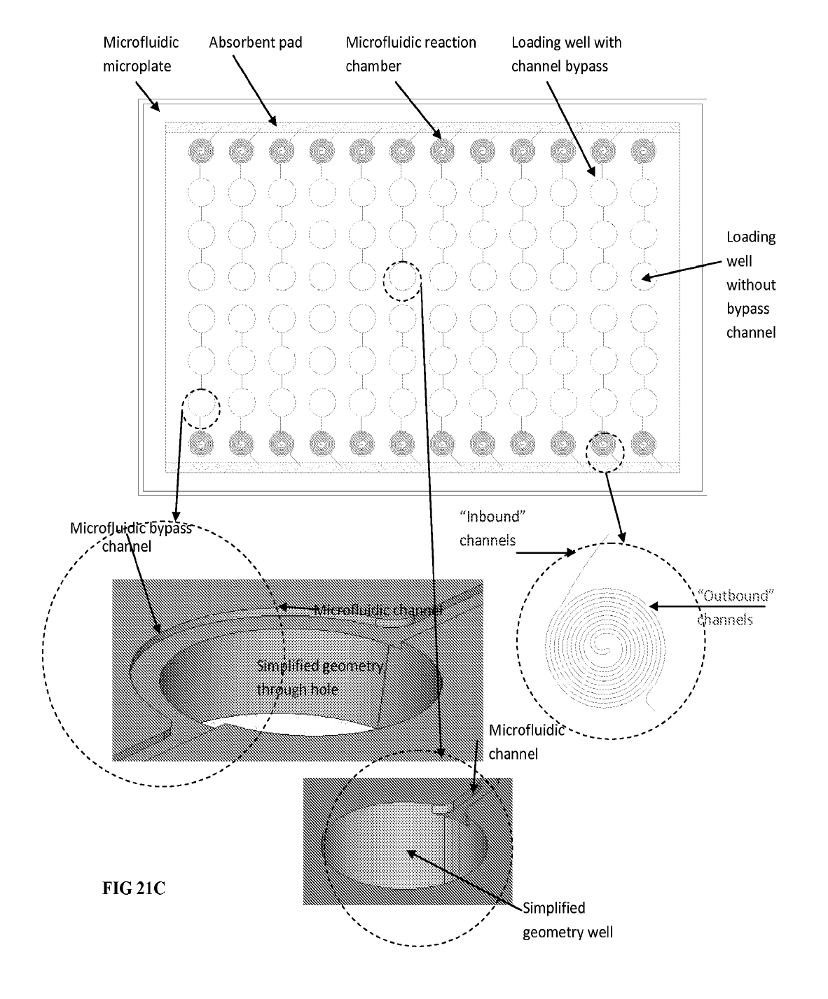

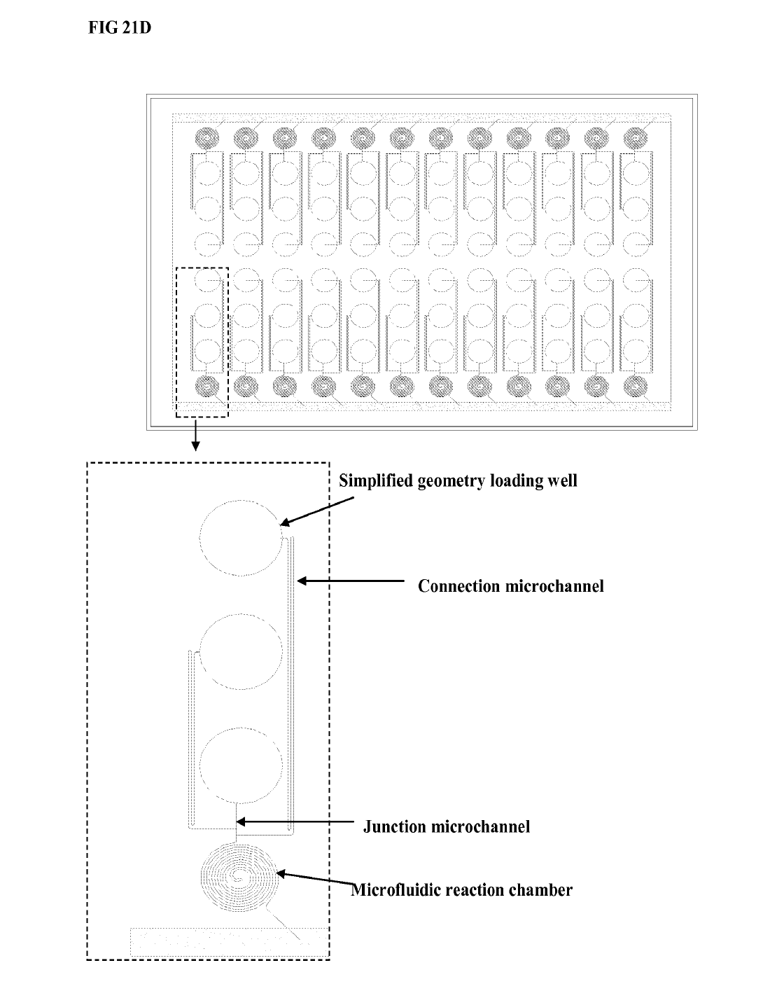

FIG. 21A shows an embodiment wherein a single loading well is connected to 1 microchannel structure directly opposite it on the other face of the substrate and to multiple other chambers which are positioned on the opposing face but in locations where other wells of the microplate would normally be present. For example, as shown in FIG. 21A, an array of 24 wells in Rows 4 and 5 are connected to 4 reaction chambers each. In one application, this device may be used for conventional assays wherein identical signals from each of the 4 reaction chambers is used for verification of assay results, as is commonly done by triplicate or more readings per sample in conventional microplate based assays. In another embodiment, the use of beads can allow for greater flexibility in the device. For example, the first liquid loaded into the common loading well could contain a bead suspension solution 1; wherein the beads are conjugated to a particular capture antibody. The volume of solution 1 is configured such that when the beads pack the most downstream reaction chamber (packing due to absorbent pad as described earlier) the beads only fill that particular microchannel structure. Then a second bead solution 2 can be added which contain beads conjugated to another antibody. These would then pack in the second from last most downstream reaction chamber and so forth. Hence, each reaction chamber can be configured to detect a different analyte from a common sample source during assay operation. Alternately, an array of different capture antibodies can be screened for sensitivity towards a common analyte or other such tests can be performed using this embodiment. It is to be appreciated, however, the embodiment may also be modified such that each reaction chamber connected in series to the loading well may have a different physical structure to ensure difference in assay characteristics.

FIG. 21B shows another embodiment wherein the loading well and the microfluidic channel are de-coupled along the vertical plane. As shown in FIG. 21B a much simplified (and higher capacity) well structure; in the form of a cylindrical structure; can be used which connects to a microfluidic channel on one side. The microfluidic channel in turn leads to the spiral (or other suitably shaped) detection region which is located in the footprint of another “well” in the standard 96-well layout. Hence, in this configuration a “96-well” configuration is reduced to a 48-well configuration but with a much simplified physical structure. Additionally, this allows for a very small thickness of plastic material on top of the spiral microfluidic channel serving as the reaction chamber. In embodiments wherein the loading well (tapered) with through hole is in the same vertical line of sight as the microchannel; there is a substantial and non-uniform thickness of plastic material above the microchannel. Specifically in fluorescence based detection applications; this increases the auto-fluorescence from the plastic material itself; since the auto-fluorescence is partially related to the thickness of the plastic material also. In the embodiment of FIG. 21B, a very small (˜250-500 μm) thickness of plastic material is allowed on the top of the microfluidic reaction chamber thereby greatly minimizing the background signal due to auto-fluorescence from plastic material itself.

FIG. 21C and FIG. 21D show embodiments that are particularly well suited for semi-automatic operation of the microfluidic microplate. FIG. 21C shows an wherein an array of simplified loading wells are connected to one reaction chamber. The schematic illustration shows the case wherein 3 loading wells are connected to one reaction chamber; and is readily apparent that this configuration can be scaled to higher number of loading wells leading to a single reaction chamber. The simplified loading wells after the first simplified loading well also use a specialized geometry for the connecting microfluidic channel as illustrated in the insert for FIG. 21C. The connection channel leading from the first simplified loading well connects with a smooth taper to the loading well. The connection channel for the other two wells loops around the base of the loading well such that a portion of the microchannel is in connection with the loading well. This geometry allows the loading well to serve a dual purpose; namely as loading well and also as an air-vent. During operation; all 3 loading wells are simultaneously filled with liquid reagents using a multi-channel pipette. Assuming a hydrophobic substrate and hydrophilic sealing tape; acknowledging that all variations outlined previously will also work equally effectively; as the 3 liquids are loaded in the wells; they will touch the base (sealing tape) and the hydrophilic forces will start drawing the liquids into the channels. In this description, the wells are described as Well 1 being the closest to the reaction chamber; Well 2 being the second upstream well and so forth. Liquid within Well 1 has an unobstructed flow path towards the reaction chamber and downstream to the absorbent pad and liquid from the Well 1 will immediately flow towards the chamber. Backflow of the liquid towards Well 2 is obstructed since there is no place for the intervening air (in the channel) to escape. Similarly liquid from Well 2 cannot flow in either direction owing to lack of an air escape path. Hence liquids in all wells other than Well 1 are “trapped” in position. As the liquid completely exits Well 1; liquid from Well 2 can start moving. The air in front of the liquid from Well 2 can escape from the now empty Well 1. Since the channel is a continuous section, and at all points is connected to the hydrophilic surface (tape); the flow will continue when liquid from Well 2 crosses the perimeter of Well 1 until the liquid from Well 2 passes through the reaction chamber and is emptied. Note that in all these cases, a narrower dimension is used for the reaction chamber to ensure that the Well is completely emptied of its contents. This sequence of flow events will continue and successive Wells (Well 3, Well 4 . . . ) reagents will be sequentially transported through the reaction chamber. By ensuring sufficient volumes (to complete the surface binding reactions) the entire assay sequence can be completed using just one load step. This embodiment offers two distinct benefits: (a) a significant reduction in labor required to run the assay sequence and (b) very reproducible results since the entire flow sequence is “automatically” regulated. Note that additional liquids can be accommodated in two ways: (a) by connecting additional wells in series (for example having 6 loading wells for a series of 5 reagents and sample that should be injected into the reaction chamber or (b) by repeating the loading sequence (for example, reagents 1, 2, and sample are injected first; then after all 3 have been transported through the reaction chamber; reagents 3, 4, and 5 are then loaded simultaneously).

FIG. 21D shows a different variant for the “semi-auto” microfluidic microplate. In this embodiment; each well drains into a channel that is connected to a common junction channel. The key difference from that shown in FIG. 21C is that the length (hence volume) of each microchannel leading up to the junction channel is significantly different. Again, using the same naming convention as the preceding example, Well 1 has a very short path length to the reaction chamber; whereas Well 2 has a path length at least 10× longer and so on. In this embodiment, as all liquids are pipetted simultaneously into their respective wells; flow will commence in all channels simultaneously. Initially, Liquid 1 (from Well 1) will reach the reaction chamber and shall be the only liquid in the reaction chamber. Thereafter, Liquid 2 (from Well 2) will reach the junction channel and a mixture of Liquid 1 and Liquid 2 will flow into the reaction chamber. The volumes of the respective Wells can be configured such that after a small volume of the mixture has passed through the reaction chamber; Well 1 is completely emptied. Thereafter, Liquid 2 alone will continue to flow through the reaction chamber until Liquid 3 (from Well 3) reaches the junction channel and so forth. This embodiment is particularly useful when two reagents should be mixed prior to loading in the reaction chamber. Examples include but are not limited to, two component chemiluminescence substrates; mixtures of labeled and sample antigens for competitive immunoassays etc. Furthermore, the flow sequence can also be configured that for a desired interval a mixture of 3 (or more) reagents is flowing simultaneously through the reaction chamber.

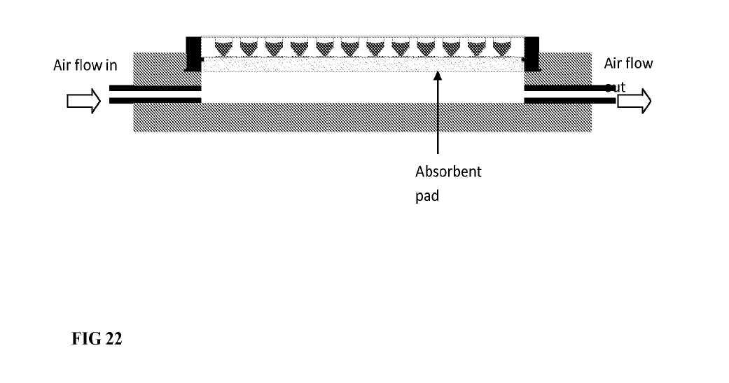

FIG. 22 shows another embodiment. In this configuration, particularly well suited for applications wherein a slow flow rate is desired for a long interval; the microplate is mounted in a special fixture. The fixture is connected to an air pump that can pump air at room temperature or elevated temperatures through the fixture which passes on the underside of the absorbent pad. The flow sequence is configured such that prior to the step where a low, steady flow rate for an extended duration is desired, a high volume of liquid is added to completely saturate the pad such that it cannot absorb any further liquid. Then the desired liquid is added to the wells and the wells are sealed on top to prevent evaporative loss, with a small air vent structure on each well seal. Furthermore, air flow is initiated in the fixture which will cause evaporative loss of liquid from the pad. As the pad loses liquid volume, additional liquid volume will be drawn from the wells at a low flow rate for an extended period of time. The absorbent pad may be a common pad for all wells or separate pads for each well. This embodiment is particularly suited for applications such as study of cell growths wherein a steady low flow of culture media is required to maintain cell viability.

The “one-body” embodiments discussed hitherto, if manufactured on a transparent substrate are not suitable for chemiluminescence based detection due to the optical cross-talk between the optically transparent wells. For fluorescence based detection, an optical signal is only generated when the microchannel with fluorescent entity is excited and after the excitation source is removed the optical signal drops to zero almost instantaneously. In the case of chemiluminescence, each microchannel unit will continuously produce a signal when the substrate is added to the channel. Hence, when a detector “reads” the channel below a given well, it will also pick up stray light signal from adjacent channels, and this “cross-talk” may lead to unacceptable errors in measurement. If an opaque substrate is used as described in some embodiments, the embodiment is suitable for chemiluminescence based detection but requires either bottom-reading mode or rotating the plate to have the channel side facing up. Most luminometers are only configured for top mode reading and the rotation step is not suitable for automation.

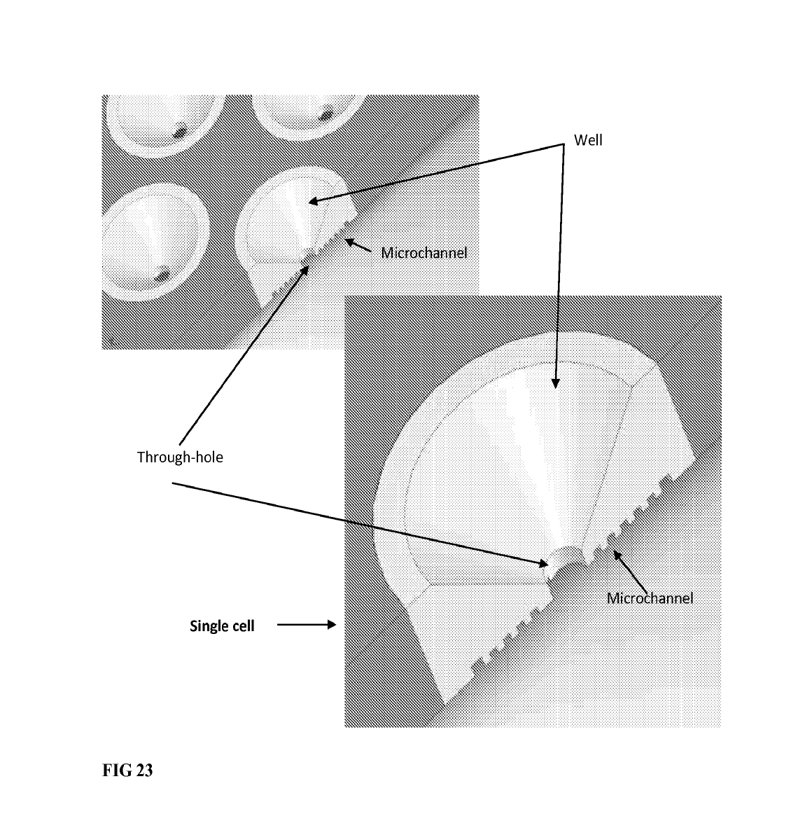

FIG. 23 shows an embodiment of the microfluidic microplate particularly well suited for chemiluminescence based detection applications. The embodiment of FIG. 23 uses a two-piece design, wherein a opaque piece is used to completely surround each well+through hole+channel “cell” of the microfluidic microplate; where each cell is composed of a transparent material. This design ensures that each cell is almost completely isolated from others where the only optical path is through the sealing tape if a continuous tape is used. If in other embodiments, each cell is also sealed individually the cells would be completely isolated from other cells. The embodiment of FIG. 23 considerably minimizes the optical cross-talk between the microfluidic microplate cells allowing for reliable chemiluminescence based detection.

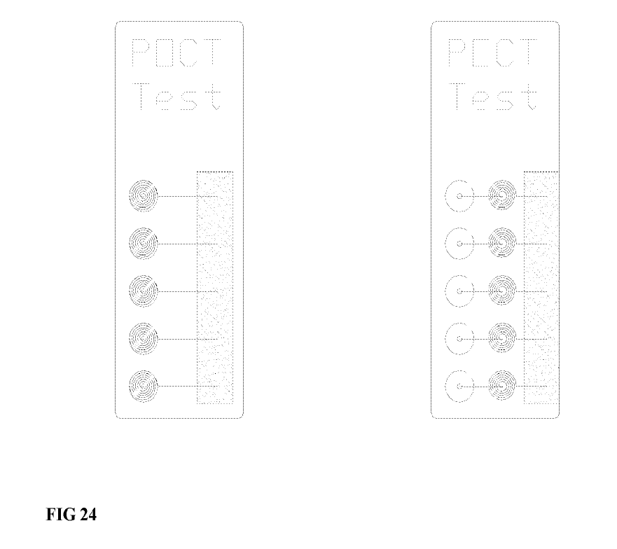

FIG. 24 shows an embodiment especially suited for point-of-care tests (POCT). This is a reduced version of the microplate and can be used as a fully manual point-of-care (POC) assay system. FIG. 24A shows a device exactly identical to the ones described earlier except with reduced number of loading/detection structures whereas FIG. 24B shows an alternate embodiment wherein the microchannel structure is not in the same vertical line of sight as the loading wells. The “semi-auto” microfluidic microplate configurations illustrated in FIGS. 21C and 21D and described previously are also well suited for a semi-auto POCT.

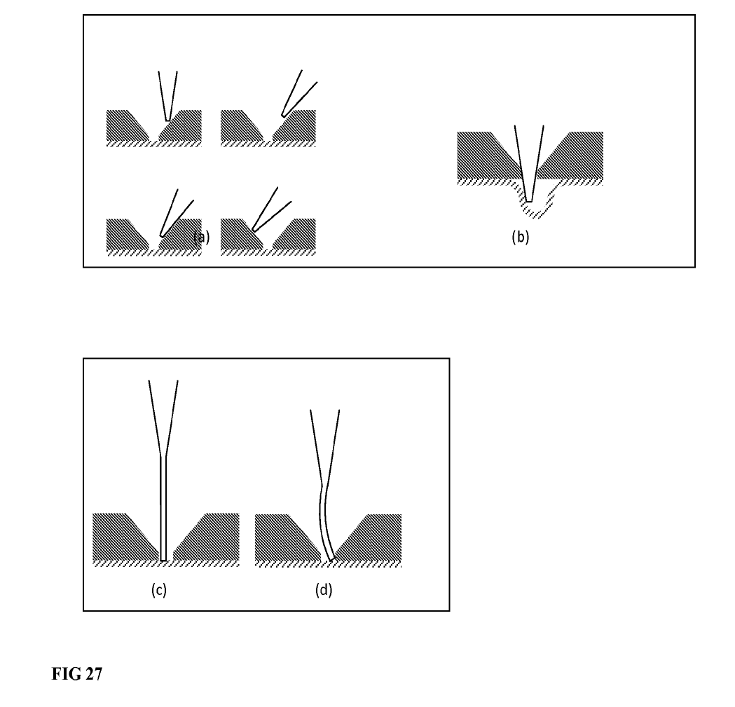

FIG. 27 shows methods of using a standard or special pipette tips for dispensing into the microfluidic microplate. Most common pipette tips are encountered in a wide range of volumetric capacity and tip dimensions. More commonly, pipette tips have either a reasonably small end diameter (at dispensing end) in the range of ˜500 μm to 1.5 mm diameter or larger end diameters of ˜1.5 mm-4 mm diameter. The small end tips are typically used for dispensing liquid only solutions; whereas the larger end diameter tips are preferred for dispensing cell suspension solutions. As described earlier, an embodiment of this invention is to selectively treat the well and through-hole such there is a continuous hydrophilic path for a liquid from the well to the hydrophilic sealing tape. In such embodiments for example, either type of pipette tips can be used to dispense the liquid or suspension solution or emulsion onto the well surface. It is not preferred to have the pipette tip positioned within the through hole, since the rigid pipette tip may push down on the hydrophilic sealing tape and break the seal between the tape and the microfluidic channel being sealed by the tape. However, in some cases, it may be advantageous to dispense within the through-hole; more specifically such that the pipette tip end is resting on the hydrophilic tape surface. This dispensing scheme allows for the liquid/suspension/emulsion to be deposited on the strongly hydrophilic tape surface and more importantly, minimizes the potential for a micro-bubble formation as the liquid swirls down from the well walls, via the through-hole on its path to the microchannel inlet. For applications wherein directly dispensing onto the hydrophilic tape is strongly preferred, we have discovered that the use of special pipette tips, including but not limited to, the so-called gel loading tips is highly preferred. Gel-loading tips have a narrow section at the dispensing end that extends a significant length (at least a few millimeters), and further wherein the diameter of said narrow section is approximately ⅕^{th} or less than the length of the section. This geometry makes the end section mechanically unstable, and if such a tip is pressed onto the sealing tape at the base of the through-hole on the microfluidic microplate cell, the end section buckles rather than breaking the seal. This is further advantageous since this scheme can also be extended for use with multi-channel pipettes. With multi-channel pipettes it is very difficult to precisely align all pipette tips to the same relative X-Y-Z position (with respect to port on the pipette body). With the mechanically weak tips, an array of such tips can be pressed down into an array of corresponding through-hole openings on the microfluidic microplate and each tip will deform unequally yet still touch the hydrophilic tape at the base. This non-intuitive use of special pipette tips may help in significantly increasing the reliability of operation for the microfluidic microplate in terms of repeatable, bubble-free dispensing.

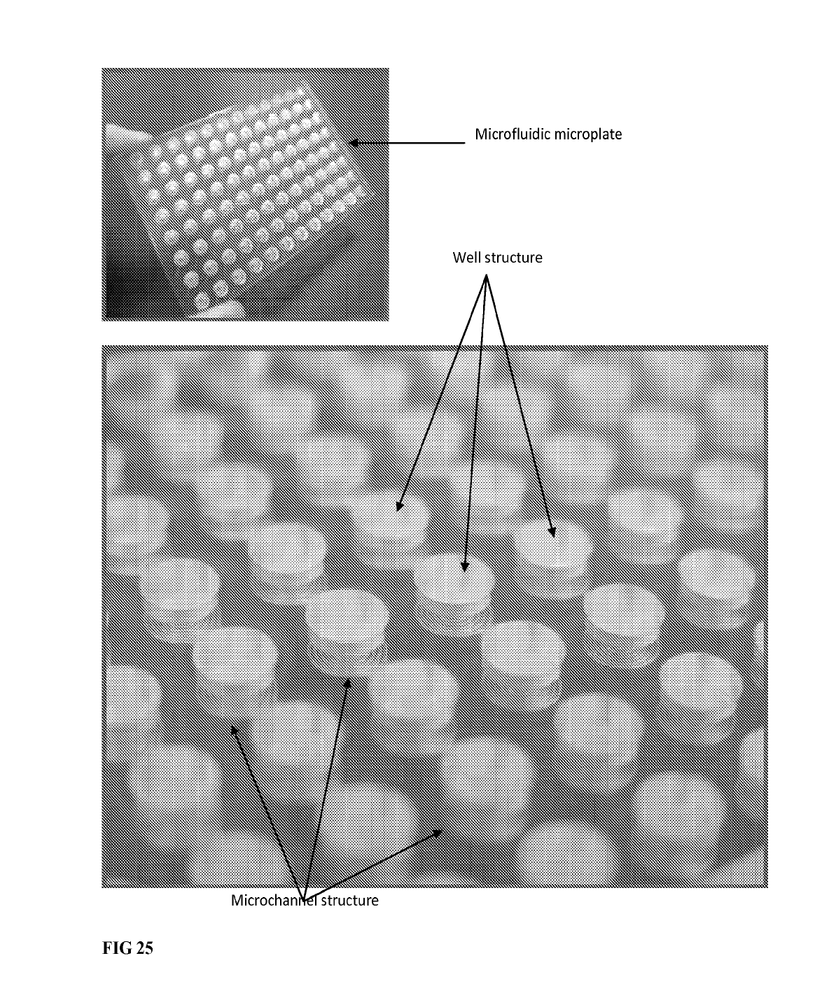

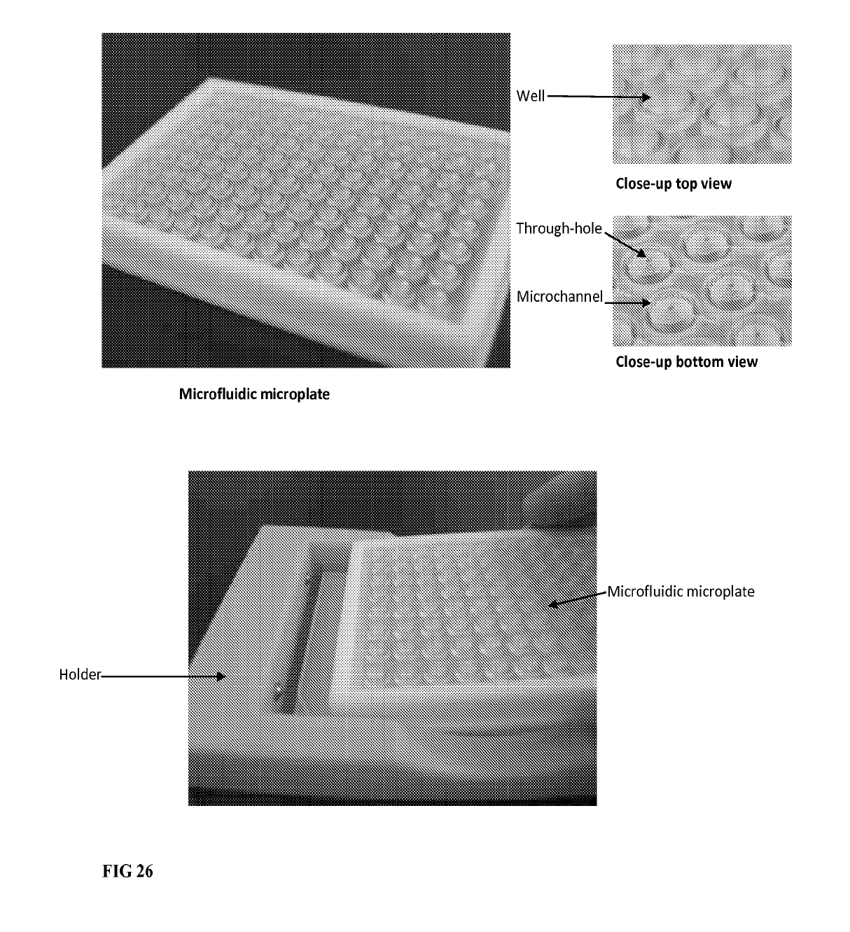

FIG. 25 shows a fabricated Optimiser™ microplate useful with the present invention with the footprint and well layout of a conventional 96 well plate, and FIG. 26 shows another embodiment of the microfluidic microplate.

The following example case study describes a detailed assay validation protocol and method comparison study to compare the performance of the Optimiser™ microplate using the principles and techniques of the invention as disclosed herein, with a conventional 96-well microplate. The example uses the IL-2 assay as an illustrative example. As described further herein, similar protocols with specific variations were used to test a range of different analytes and the data is summarized further in this disclosure. References are made by trade names and trademarks to commercially available material and reagents utilized in the following example.

### Case Study: Il-2 Assay Using Low Sample Volume

### Materials and Equipment

Siloam Biosciences, Inc. Optimiser™ Microplate System, Cat #96FX-1/1-X
Purified anti-mouse IL-2 antibody, 0.5 mg/ml, clone JES6-1A12, for ELISA Capture
Recombinant mouse IL-2 protein, 0.01 mg/ml, calibrated for ELISA standard
Biotinylated anti-mouse IL-2 antibody, 0.5 mg/ml, clone JES6-5H4, for ELISA Detection
Streptavidin-Horseradish Peroxidase (SAv-HRP), KPL, 0.5 mg/ml, Cat#14-30-00

### 1-Step Ultra TMB-ELISA Absorbance Substrate, Pierce, Cat#34028

2N sulfuric acid; Stop Solution for TMB Substrate
Siloam Biosciences, Inc. QuantaRed™ Enhanced Chemifluorescent HRP Substrate Kit, Pierce, Cat#15159
Siloam Biosciences, Inc. OptiPrime™ Pre-Wetting Solution
Siloam Biosciences, Inc. OptiCoat™ Coating Buffer
Siloam Biosciences, Inc. OptiWash™ Wash Buffer
Siloam Biosciences, Inc. OptiBlock™ Blocking Buffer
RPMI-1640 medium, 10×, Sigma, Cat# R1145

### Fetal Bovine Serum, Sigma, Cat# F2442

Pooled normal mouse serum, Innovative Research
BioTek FLx800 Fluorescence Microplate Reader, using 528/20 nm excitation filter and 590/35 nm emission filter, with sensitivity set at 45

### Awareness Technology ChroMate® Absorbance Microplate Reader (OD450 nm)

NUNC high protein-binding capacity 96-Well plate, PS, MaxiSorp®, Flat, Clear, for absorbance detection
VWR Vacuum Filtration system, 500 ml, 0.2 μm PES Membrane
96-well polypropylene conical bottom plate

### Reagent and Plate Preparation

### 1) Coating Buffer: OptiCoat™ Coating Buffer

### 2) Blocking Buffer: OptiBlock™ Blocking Buffer

### 3) Wash Buffer: OptiWash™ Wash Buffer

4) Capture Antibody Solution: Purified anti-mouse IL-2 antibody diluted to 2 μg/ml with Coating Buffer
5) Cell Culture Medium: 10% FBS in 1×RPMI medium, pH adjusted to 7, filtered with 0.2 μm vacuum filtration system
6) Mouse Serum: Normal mouse serum centrifuged at 13,000 g for 10 minutes and supernatant harvested.
7) Assay Standards: Recombinant mouse IL-2 protein diluted to 1.0 ng/ml with appropriate matrices, and then serially-diluted (2-fold) into the 96-well conical bottom plate with matrices. Eleven concentrations of IL-2-spiked standard were prepared, over a range from 1.0 ng/ml to 1.0 pg/ml. Non-spiked matrices used as a zero point.
8) Detection Antibody Solution: Biotinylated anti-mouse IL-2 antibody diluted to 2 μg/ml with Blocking Buffer
9) SAv-HRP: HRP conjugated streptavidin diluted to a) 0.125 μg/ml (1:4000) with Blocking Buffer for Optimiser™ and b) 0.25 μg/ml (1:2000) for conventional 96-well microplate. Sodium azide is excluded from all buffers, as this interferes with HRP activity.
10) Chemifluorescent Substrate Final (Working) Solution: Equilibrate the QuantaRed™ substrate kit to room temperature for at least 10 minutes. Mix 50 parts QuantaRed™ Enhancer Solution with 50 parts QuantaRed™ Stable Peroxide and 1 part QuantaRed™ ADHP Concentrate. Use within 30 minutes after preparation.
11) Absorbance Substrate (for conventional 96-well assay): Equilibrate the TMB substrate to room temperature before use.
12) Priming: Assemble Optimiser™ microplate with absorbent pad and holder, load 10 μl of the supplied PBS based priming buffer solution into each well of the Optimiser™ plate, and wait until all wells are empty. Use the plate within 15 minutes.

Working concentrations for capture antibody, detection antibody were optimized by following the NIH Guidance for immunoassay development.

### Assay Procedure

Both cell culture medium and mouse serum were used in this assay to demonstrate the compatibility of the Optimiser™ to measuring analytes in complex biological fluids, and to validate the performance of the Optimiser™ versus conventional ELISA formats. The same IL-2 sandwich immunoassay was performed in two different assay platforms:

1) Clear Conventional High Protein-Binding Capacity 96-well plate for absorbance detection (OD450/630)
2) Optimiser™ Microfluidic Plate for chemifluorescence detection (528/590 nm)

The Optimiser™ Microplate assay procedure is described here. The Clear Conventional High Protein-Binding Capacity 96-well plate for absorbance detection assay procedure is described in Case Study Appendix A-2. The total assay time to run the Optimiser™ plate is about 1 hour, which is only 1/10 the time requirement for a conventional 96-well ELISA (˜5-18 hours)

### Assay Steps

Ensure that the Optimiser™ priming procedure as described in Step 12 of Reagent and Plate Preparation section is completed before starting the assay procedure.

1) Assemble Optimiser™ microplate with absorbent pad and holder. Prime the plate before starting the assay.
2) Add 10 μl of Capture Antibody Solution into each well, and incubate at room temperature for 5 minutes.
3) Add 10 μl of Blocking Buffer into each well, and incubate at room temperature for 5 minutes.
4) Pipette 10 μl of each Assay Standard into appropriate wells in triplicate rows, and incubate at room temperature for 10 minutes.
5) Add 30 μl of Wash Buffer into each well; wait until all wells are empty.
6) Add 10 μl of Detection Antibody Solution into each well, and incubate at room temperature for 10 minutes.
7) Repeat step 5.
8) Add 10 μl of SAv-HRP Solution into each well, and incubate at room temperature for 10 minutes.
9) Change the absorbent pad
10) Repeat step 5, twice.
11) Add 10 μl of QuantaRed™ Working Solution in each well, wait until all wells are empty, and take off the plate from the holder. Wipe off all residue from bottom of Optimiser™ plate with Kimwipe®. Set Fluorescence Microplate Reader for fluorescence excitation wavelength of 528 nm and fluorescence emission wavelength of 590 nm (with sensitivity set at 45). Measure the fluorescence at the time point 15 minutes after adding substrate.

### Calculation of Results

Calculate the mean value of each set of triplicate samples. Subtract the mean value of blanks (zero point) from each.

Create a standard curve by reducing the data using computer software capable of generating a four parameter logistic (4-PL) curve fit. As an alternative, plot the curve on log-log graph, with IL-2 concentration on x-axis, and signal reading on the y-axis. A best-fit curve is drawn through the points of each assay.

### Results and Conclusions

### Cell Culture Medium

The results demonstrate linearity for assays on both the Optimiser™ and conventional 96-well plate, by using cell culture medium as matrix over the dynamic range of 250 pg/ml to 2.0 pg/ml. The raw data is shown in Table CS1. The calculated results and standard curves are shown in Figure CS1a) and CS1b).

TABLE CS1
Signal readings from IL-2 sandwich assays using spiked
cell culture medium samples, in triplicate:
a) NUNC 96-well plates, Absorbance, OD at 450 nm (subtracting
630 nm), and b) Optimiser ™ plates, Chemifluorescence,
RLU at 528/590 nm, reader sensitivity at 45.

| (a) | | | | (b) | | | |
|---|---|---|---|---|---|---|---|
| NUNC Colorimetric Assay | | | | Optimiser ™ Chemifluorescent Assay | | | |
| IL-2 | | | | IL-2 | | | |
| (pg/ml) | OD1 | OD2 | OD3 | (pg/ml) | FL1 | FL2 | FL3 |
| 250 | 2.025 | 2.593 | 2.796 | 250 | 5373 | 6498 | 4622 |
| 125 | 1.287 | 1.427 | 1.603 | 125 | 3146 | 2814 | 2973 |
| 63 | 0.608 | 0.695 | 0.911 | 63 | 1940 | 1677 | 1344 |
| 31 | 0.339 | 0.447 | 0.46 | 31 | 1090 | 1012 | 921 |
| 16 | 0.177 | 0.231 | 0.231 | 16 | 663 | 504 | 633 |
| 8 | 0.091 | 0.131 | 0.139 | 8 | 462 | 480 | 482 |
| 4 | 0.054 | 0.077 | 0.081 | 4 | 401 | 325 | 430 |
| 2 | 0.033 | 0.041 | 0.048 | 2 | 308 | 245 | 299 |
| 0 | 0.014 | 0.016 | 0.014 | 0 | 250 | 244 | 230 |

Below in graphical and table form are Case Study 1 data for an IL-2 assay tested with spiked cell culture media in the Optimiser™ microplate and comparative data for the same assay on a 96-well plate.

Standard curve of IL-2 assay using spiked cell culture medium samples run in conventional 96-well plate, using TMB substrate and colorimetric detection of absorbance at 450 nm (subtracting 630 nm).

Standard curve of IL-2 assay using spiked cell culture medium samples run in Siloam Biosciences Optimiser™ microplate, using QuantaRed™ substrate for chemifluorescence detection.

### Mouse Serum

The results demonstrate linearity for assays on both the Optimiser™ and conventional 96-well plate, by using by using mouse serum as matrix, over the dynamic range of 250 pg/ml to 2.0 pg/ml. The raw data is shown below in the table. The calculated results and standard curves are shown in the following tables and graphs.

Signal readings from IL-2 sandwich assays using spiked mouse serum samples, in triplicate:

a) NUNC 96-well plates, Absorbance, OD at 450 nm (subtracting 630 nm), and b) Optimiser™ plates, Chemifluorescence. RLU at 528/590 nm, reader sensitivity at 45.

| (a) | | | | (b) | | | |
|---|---|---|---|---|---|---|---|
| NUNC Colorimetric Assay | | | | Optimiser ™ Chemifluorescent Assay | | | |
| IL-2 | | | | IL-2 | | | |
| (pg/ml) | OD1 | OD2 | OD3 | (pg/ml) | FL1 | FL2 | FL3 |
| 250 | 1.782 | 2.465 | 2.336 | 250 | 4960 | 4883 | 3917 |
| 125 | 1.168 | 1.306 | 1.162 | 125 | 2653 | 2787 | 2528 |
| 63 | 0.649 | 0.773 | 0.71 | 63 | 1449 | 1611 | 1385 |
| 31 | 0.393 | 0.439 | 0.47 | 31 | 743 | 677 | 729 |
| 16 | 0.248 | 0.257 | 0.277 | 16 | 450 | 500 | 614 |
| 8 | 0.192 | 0.181 | 0.179 | 8 | 257 | 386 | 292 |
| 4 | 0.177 | 0.15 | 0.144 | 4 | 182 | 222 | 213 |
| 2 | 0.141 | 0.139 | 0.138 | 2 | 186 | 181 | 181 |
| 0 | 0.053 | 0.139 | 0.135 | 0 | 171 | 148 | 159 |

The following are summaries of Case Study 1 data for an IL-2 assay tested with mouse serum in the Optimiser™ microplate, and comparative data for the same assay on a conventional 96-well plate.

Standard curve of IL-2 assay using spiked mouse serum samples run in conventional 96-well plate, using TMB substrate and colorimetric detection of absorbance at 450 nm (subtracting 630 nm).

Standard curve of IL-2 assay using spiked mouse serum samples run in Siloam Biosciences Optimiser™ microplate, using QuantaRed™ substrate for chemifluorescence detection.

### Performance Characteristics

Five different levels of IL-2 were spiked into five sample replicates throughout the range of the assay in various matrices. Calculations were performed as follows:

Calculate the concentrations of the validation samples of each run using the respective calibration curves. Then compute the % recovery of those validation samples using the following formula:

% Recovery=100×(Estimated concentration)/True concentration

Calculate the average and standard deviation of the calculated data of the validation samples for each concentration. Then compute the % precision (CV) of these validation samples using the following formula:

% precision=100×(Standard deviation)/Calculated concentration

TABLE
Assay performance characteristics with cell culture medium as matrix
Conventional 96-well Microplate

| Sample | 1 | 2 | 3 | 4 | 5 |
|---|---|---|---|---|---|
| n | 5 | 5 | 5 | 5 | 5 |
| Expected value (pg/ml) | 188 | 93.8 | 46.9 | 23.4 | 11.7 |
| Average of calculated value | 164 | 95.4 | 50.7 | 28.3 | 13.7 |
| (pg/ml) | | | | | |
| Standard deviation | 16.9 | 10.4 | 6.1 | 2.2 | 2.0 |
| Precision/CV (%) | 10 | 11 | 12 | 8 | 14 |
| Recovery (%) | 88 | 102 | 108 | 121 | 117 |

| | |
|---|---|
| Average Precision | 11% |
| Average Recovery | 107% |

### Optimiser™ Microplate

| Sample | 1 | 2 | 3 | 4 | 5 |
|---|---|---|---|---|---|
| n | 5 | 5 | 5 | 5 | 5 |
| Expected value (pg/ml) | 188 | 93.8 | 46.9 | 23.4 | 11.7 |
| Average of calculated value | 164 | 89.0 | 45.4 | 27.6 | 12.4 |
| (pg/ml) | | | | | |
| Standard deviation | 27.2 | 4.1 | 2.5 | 1.0 | 1.8 |
| Precision/CV (%) | 17 | 5 | 5 | 3 | 15 |
| Recovery (%) | 87 | 95 | 97 | 118 | 106 |

| | |
|---|---|
| Average Precision | 9% |
| Average Recovery | 101% |

TABLE
Assay performance characteristics with mouse serum as matrix
Conventional 96-well Microplate

| Sample | 1 | 2 | 3 | 4 | 5 |
|---|---|---|---|---|---|
| n | 5 | 5 | 5 | 5 | 5 |
| Expected value (pg/ml) | 188 | 93.8 | 46.9 | 23.4 | 11.7 |
| Average of calculated value | 185 | 102.3 | 54.4 | 28.8 | 13.5 |
| (pg/ml) | | | | | |
| Standard deviation | 17.9 | 7.5 | 5.4 | 3.5 | 1.5 |
| Precision/CV (%) | 10 | 7 | 10 | 12 | 11 |
| Recovery (%) | 99 | 109 | 116 | 123 | 115 |

| | |
|---|---|
| Average Precision | 10% |
| Average Recovery | 112% |

### Optimiser™ Microplate

| Sample | 1 | 2 | 3 | 4 | 5 |
|---|---|---|---|---|---|
| n | 5 | 5 | 5 | 5 | 5 |
| Expected value (pg/ml) | 188 | 93.8 | 46.9 | 23.4 | 11.7 |
| Average of calculated value | 136 | 80.6 | 42.0 | 26.1 | 14.2 |
| (pg/ml) | | | | | |
| Standard deviation | 35.3 | 11.3 | 6.1 | 2.9 | 2.4 |
| Precision/CV (%) | 26 | 14 | 15 | 11 | 17 |
| Recovery (%) | 73 | 86 | 90 | 111 | 122 |
| Average Precision | 17% | | | | |
| Average Recovery | 96% | | | | |

Benefits and Unique Advantages of the Optimiser™ ELISA in Accordance with the Present Invention

In this IL-2 assay example, the Optimiser™ Microplate System clearly demonstrates the following dramatic benefits and advantages, in comparison with conventional high protein-binding capacity 96-well plates:
10 fold less precious experimental sample
10 fold less reagent (Capture Ab, Detection Ab, SAv-HRP, Substrate); hence 10 fold reduction in reagent costs
30-60 minute total assay time (10 fold less than conventional)
Convenient use of partial plate assays
Equivalent sensitivity
Equivalent dynamic range
No washes or plate washer required
Precludes “edge effect” of conventional plates
Ultimate speed and convenience for immunoassay applications
Dramatically-reduced hands-on time
Equivalent performance using complex biological fluids, such as serum and plasma

### Cost Savings Summary for IL-2 Assay

| | | Traditional 96 | | | Optimiser ™ | | |
|---|---|---|---|---|---|---|---|
| | | Well Plate | | | Plate | | |
| | Unit | Usage/ | | Total | Usage/ | | Total |
| Reagent | price | plate | | cost | plate | | cost |
| Capture antibody | $0.44/μg | 20 | μg | $8.8 | 2.0 | μg | $0.88 |
| Detection antibody | $0.52/μg | 20 | μg | $10.4 | 2.0 | μg | $1.04 |
| HRP conjugate | $0.20/μg | 2.5 | μg | $0.5 | 0.125 | μg | $0.025 |
| Blocking buffer | $0.15/ml | 60 | ml | $9 | 4 | ml | $0.6 |
| Standard diluents- | $2.00/ml | 3 | ml | $6 | 0.5 | ml | $1.5 |
| Serum | | | | | | | |

| | | | | | | |
|---|---|---|---|---|---|---|
| Substrate | $3.00/ml | — | — | 1 | ml | $3 |
| (QuantaRed) | | | | | | |

| | | | | | | |
|---|---|---|---|---|---|---|
| Substrate | $0.50/ml | 10 | ml | $5 | — | — |
| (Ultra-TMB) | | | | | | |

### Performance and Assay Reagent Cost Comparison (Per Plate)

| | Conventional 96-well | Optimiser ™ |
|---|---|---|
| Minimum detectable | 2 pg/ml (0.2 pg/well) | 2 pg/ml (0.02 pg/well) |
| dose | | |
| Cost | $40 | $7 |

Case Study 1: Il-2 Assay Optimization with Optimiser™ Plate

Before performing an assay with experimental samples, as with all antibody-based assays, the reagents should be titrated to determine the best working concentrations for use in the Optimiser™ plate. Following is the example procedure to determine the best working concentrations of detection antibody and HRP conjugate by following the NIH Guidance for Immunoassay Development¹. The same IL-2 assay optimization was performed in two different assay platforms:

1) Clear Conventional High Protein-Binding Capacity 96-well plate for absorbance detection (OD450/630)
2) Optimiser™ Microfluidic Plate for chemifluorescence detection (528/590 nm).

### Reagent Preparation

1) Coating Buffer: OptiCoat™ Coating Buffer
2) Blocking Buffer: OptiBlock™ Blocking Buffer
3) Wash Buffer: OptiWash™ Wash Buffer
4) Capture Antibody Solution: Purified anti-mouse IL-2 antibody was diluted to 8, 4, 2 and 1 μg/ml with Coating Buffer
5) Assay Standards: Recombinant mouse IL-2 protein was diluted to 1000 and 20 pg/ml with Blocking Buffer. The non-spiked matrices were used as the zero point.
6) Detection Antibody Solution: Biotinylated anti-mouse IL-2 antibody was diluted to 8, 4, 2 and 1 μg/ml with Blocking Buffer.
7) SAv-HRP: HRP conjugated streptavidin was diluted to a) 0.125 μg/ml (1:4000) with Blocking Buffer for Optimiser and b) 0.25 μg/ml (1:2000) for conventional 96-well. Sodium azide is excluded from all buffers used for reagents, as this interferes with HRP activity.
8) Chemifluorescent Substrate Final Working Solution (For Optimiser™ assay): Equilibrate the QuantaRed™ substrate kit to room temperature for at least 10 minutes. Mix 50 parts QuantaRed™ Enhancer Solution with 50 parts QuantaRed™ Stable Peroxide and 1 part QuantaRed™ ADHP Concentrate. Use within 30 minutes after preparation.
9) Absorbance Substrate (For conventional 96-well assay): Equilibrate the TMB substrate to room temperature before use.
10) Optimiser™ Priming: Assemble Optimiser™ microplate with absorbent pad and holder, load 10 μl of Opti-Prime solution into each well of the Optimiser™ plate, wait until all wells are empty, use the plate within 15 minutes.

### Experimental Procedure

The Optimiser™ Microplate assay procedure is described here. The clear conventional high protein-binding capacity 96-well plate for absorbance detection assay procedure is described in Appendix A-2.

Ensure that the Optimiser™ priming procedure as described in Step 10 of Reagent and Plate Preparation section is completed before starting the assay procedure.
1) Add 10 μl of Capture Antibody Solution into appropriate wells (see Plate Layout), and incubate at room temperature for 5 minutes.
2) Add 10 μl of Blocking Buffer into each well, and incubate at room temperature for 5 minutes.
3) Pipette 10 μl of each Assay Standard into appropriate wells, and incubate at room temperature for 10 minutes.
4) Add 30 μl of Wash Buffer into each well; wait until all wells are empty.
5) Add 10 μl of Detection Antibody Solution into appropriate wells, and incubate at room temperature for 10 minutes.
6) Repeat step 5.
7) Add 10 μl of SAv-HRP Solution into each well, and incubate at room temperature for 10 minutes.
8) Change the absorbent pad.
9) Repeat step 5, twice.
10) Add 10 μl of QuantaRed™ Working Solution in each well, wait until all wells are empty, and take off the plate from the holder. Wipe off all residue from bottom of the Optimiser™ plate with Kimwipe®, measure the fluorescence at time point of 15 minutes after adding substrate. Set Fluorescence Microplate Reader for fluorescence excitation wavelength of 528 nm and fluorescence emission wavelength of 590 nm (with sensitivity set at 45).

### Plate Layout

| Detection | Capture antibody | | | | | | | | | | | |
|---|---|---|---|---|---|---|---|---|---|---|---|---|
| antibody | 8 μg/mL | | | 4 μg/mL | | | 2 μg/mL | | | 1 μg/mL | | |
| 8 μg/mL | 1000 pg/ml | 20 pg/ml | 0 | 1000 pg/ml | 20 pg/ml | 0 | 1000 pg/ml | L | 0 | 1000 pg/ml | 20 pg/ml | 0 |
| | 1000 pg/ml | 20 pg/ml | 0 | 1000 pg/ml | 20 pg/ml | 0 | 1000 pg/ml | L | 0 | 1000 pg/ml | 20 pg/ml | 0 |
| 4 μg/mL | 1000 pg/ml | 20 pg/ml | 0 | 1000 pg/ml | 20 pg/ml | 0 | 1000 pg/ml | L | 0 | 1000 pg/ml | 20 pg/ml | 0 |
| | 1000 pg/ml | 20 pg/ml | 0 | 1000 pg/ml | 20 pg/ml | 0 | 1000 pg/ml | L | 0 | 1000 pg/ml | 20 pg/ml | 0 |
| 2 μg/mL | 1000 pg/ml | 20 pg/ml | 0 | 1000 pg/ml | 20 pg/ml | 0 | 1000 pg/ml | L | 0 | 1000 pg/ml | 20 pg/ml | 0 |
| | 1000 pg/ml | 20 pg/ml | 0 | 1000 pg/ml | 20 pg/ml | 0 | 1000 pg/ml | L | 0 | 1000 pg/ml | 20 pg/ml | 0 |
| 1 μg/mL | 1000 pg/ml | 20 pg/ml | 0 | 1000 pg/ml | 20 pg/ml | 0 | 1000 pg/ml | L | 0 | 1000 pg/ml | 20 pg/ml | 0 |
| | 1000 pg/ml | 20 pg/ml | 0 | 1000 pg/ml | 20 pg/ml | 0 | 1000 pg/ml | L | 0 | 1000 pg/ml | 20 pg/ml | 0 |

### Conclusion

Based on the experiment results, 2 μg/ml of capture antibody and 2 μg/ml of detection antibody were selected as the optimal antibody concentrations to perform the IL-2 assay in both the Optimiser™ and with conventional 96-well microplates, yielding excellent signal to noise ratios, as well as low reagent consumption.

### Case Study 1: Il-2 Assay Procedure for Conventional 96-Well Plate

1) Add 100 μl Capture Antibody Solution into each well, seal plate, and incubate at 37° C. for 1.5 hours.
2) Wash the plate with PBS (T-20), 2 times and followed by PBS, 3 times.
3) Add 300 μl Blocking Buffer into each well, seal plate, and incubate at 37° C. for 1.5 hours.
4) Repeat Step 2.
5) Pipette 100 μl of each prepared standards, controls and/or samples into appropriate wells, seal the plate with film, and incubate at 37° C. for 1.5 hours.
6) Repeat Step 2.
7) Add 100 μl of Detection Antibody Solution into each well, seal the plate with film, and incubate at 37° C. for 1.5 hours.
8) Repeat Step 2.
9) Add 100 μl of SAv-HRP Solution into each well, seal the plate with film, and incubate at 37° C. for 1.5 hours.
10) Repeat Step 2.
11) Add 100 μl of the Ultra-TMB Substrate Solution per well, incubate plate at room temperature for 15 minutes, stop reaction by adding 50 μl of 2 N sulfuric acid to each well, measure the absorbance of each well at 450 nm and 630 nm, subtract 630 nm values from at 450 nm values.

Case study 1 illustrates that the Optimiser™ device and methods in accordance with the present invention offer distinct advantages for immunoassay based analysis techniques by comparison with conventional assay devices and methods. Specifically, Case Study 1 illustrates the significant sample volume and assay time savings made possible by use of the Optimiser™ for the IL-2 assay. As described earlier, the Case study is only an illustrative example and similar performance benefits (to varying degree) can be achieved for other assays as shown in the Table 2 below.

TABLE
Limit of Detection (LOD) and Limit of Quantification (LOQ) comparison with
conventional 96-well microplate when Optimiser ™ uses only 10 μl sample volume
(conventional 96-well microplate experiment used 100 μl sample volume)

| | | | Human | | | |
|---|---|---|---|---|---|---|
| | | Human IL-4 | IL-6 | Mouse IL-2 | Mouse IFN-gamma | Mouse IL-17A |
| Capture antibody | | 8D4-8 | MQ2-13A5 | JES6-1A12 | AN-18 | TC11-18H10.1 |
| Detection antibody | | MP4-25D2 | MQ2-39C3 | JES6-5H4 | R4-6A2 | TC11-8H4 |
| Antigen | | eBioscience, | eBioscience, | eBioscience, | BD | eBioscience, |
| | | 14-8049-62 | 14-8069-62 | 14-8021-64 | Biosciences, | 14-8171-62 |
| | | | | | 554587 | |
| LOD | conventional | 0.49 | 1.9 | 1.9 | 7.8 | 7.8 |
| (pg/mL) | Optimiser, | 0.98 | 0.98 | 1.9 | 3.9 | 15 |
| | 10 μl | | | | | |
| LOQ | conventional | 1.5 | 2.9 | 2.9 | 12 | 23 |
| (pg/mL) | Optimiser, | 1.5 | 2.9 | 2.9 | 23 | 23 |
| | 10 μl | | | | | |

| | | | | | |
|---|---|---|---|---|---|
| Better in pre-wetting? | No | No | Yes | Yes | Yes |
| Incubation time | 10 min | 10 min | 10 min* | 10 min* | 10 min* |

*for IL-2, IFN-gamma, IL-17A, increase incubation time to 20 min will improve precision LOD and LOQ as defined by NIH Assay Guidance Manual of Immunoassay Methods, http://www.ncgc.nih.gov/guidance/section10.html.

Each assay preferably should be optimized on the Optimiser™ and offers different levels of sensitivity when compared to the conventional 96-well microplate. Generally, the Optimiser™ shows similar sensitivity (LOD and LOQ) as the conventional microplate. However, during assay optimization experiments we have identified 2 key parameters that vary based on the assay type:
The incubation intervals for each assay is not the same: some assays (for example the IL-4 assay) show near saturation response with ˜10 minute incubation cycles whereas other assays such as the IL-2 show good sensitivity only with 20 minute (or even longer) incubation steps. The optimum assay time needs to be established assay by assay.
As described earlier herein, different assays exhibit different behavior to the pre-wetting solution. As summarized in Table 2 above and further illustrated in the Table 3 below; each assay shows a significantly different performance improvement when the pre-wetting step is added to the assay sequence. Logically, this leads to the inference that compositions of the pre-wetting buffer (the aforementioned PBS based priming buffer) may lead to substantial improvements in assay performance on an assay-by-assay basis.

TABLE
Pre-wetting comparison data:

| | Human | Human | Mouse | Mouse IFN- | Mouse |
|---|---|---|---|---|---|
| | IL-4 | IL-6 | IL-2 | gamma | IL-17A |
| S/N* without | 41 | 45 | 41 | 42 | 42 |
| pre-wetting | | | | | |
| S/N with | 41 | 44 | 23 | 15 | 16 |
| pre-wetting | | | | | |

*S/N = Signal of highest suggested detectable concentration/Signal of background (zero)

Another factor that distinguishes assay performance on the Optimiser™ by comparison with conventional devices and methods is the effect of the sample matrix on the assay results. The measurement of analytes in serum (or plasma) matrices by sandwich ELISA can be confounded by naturally-occurring interfering factors which can cross link capture and detection antibodies, yielding distinct false positives. Such naturally-occurring interfering activities are often attributed to rheumatoid factor or HAMA (Human Anti-Mouse Antibody)-like effects in the serum (or plasma) samples. Rh factor, an autoantibody reactive with the Fc portion of IgG, is often identified in patients suspected of having arthritis, but, notably, this activity is observed in 5-10% of healthy persons, leading to false positive signals. This effect is well characterized for 96-well ELISA based analysis. However, the Optimiser™ shows significantly improved performance even with the same serum/plasma factors for some of the assays. This In order to illustrate this, multiple antibody sets for various assays were tested with both human (serum and plasma) and mouse (serum) matrices. It was found that the Optimiser™ performance varies widely on an assay-by-assay basis and can be used as means to optimize certain assays for a given matrix.

It will be appreciated by those skilled in the art that the present invention provides an extremely versatile immunoassay system that enables the end-user to “tune” the assay to their desired requirements. One example of this is the use of higher sample volumes to increase the sensitivity of the assay. The loading well of the Optimiser™ is configured to contain ˜30 μl liquid volume. Volumes less than 30 μl can be added to the Optimiser™ for any assay step as a single load from a pipette. Volumes higher than 30 μl can be added by repetitive loads; for example 90 μl sample can be added as 3 loads of 30 μl. This can be done either in the manual mode when an operator runs the Optimiser™ microplate; or with an automation system. The key difference between the manual and automation mode of use is the number of repeat sample volume loads and the volume loaded in each step. Generally, it is not feasible for an operator to manipulate very small volumes (˜5 μl or less) or perform large number of repeat loads (>5 sample loads). These tasks are better suited for automation based approaches where an automated liquid handler will perform the necessary reagent dispense steps in precisely timed intervals. The low-volume handling or large number of repetitive loads is certainly not impossible for an operator and can be accomplished by careful and meticulous attention to operation of the liquid handling steps for the assay. Table 6 shows the effects of increasing sample volumes on the assay detection limits in the manual mode. These experiments used a similar experimental protocol as described in Case Study 1 with the following exceptions:
10 μl case: single load of sample volume followed by 10 minute incubation cycle
30 μl case: single load of sample volume followed by 10 minute incubation cycle
90 μl case: 3 loads of sample volume (30 μl each) followed by 10 minute incubation cycle after each load step.

TABLE
LOD/LOQ comparison for 10 μl, 30 μl and 90 μl sample
volumes on Optimiser ™ compared with 100 μl sample
volume on the conventional 96-well microplate

| | | | | | Mouse |
|---|---|---|---|---|---|
| | | Human | Human | Mouse | IFN- |
| | | IL-4 | IL-6 | IL-2 | gamma |
| Capture | | 8D4-8 | MQ2-13A5 | JES6-1A12 | AN-18 |
| antibody | | | | | |
| Detection | | MP4-25D2 | MQ2-39C3 | JES6-5H4 | R4-6A2 |
| antibody | | | | | |
| Antigen | | eBio- | eBio- | eBio- | BD |
| | | science, | science, | science, | Bio- |
| | | 14-8049-62 | 14-8069-62 | 14-8021-64 | sciences, |
| | | | | | 554587 |
| LOD | Conven- | 0.49 | 1.9 | 1.9 | 7.8 |
| (pg/ | tional | | | | |
| mL) | 10 μL | 0.98 | 0.98 | 1.9 | 3.9 |
| | 30 μL | 0.49 | 0.98 | 0.98 | 1.9 |
| | 90 μL | 0.24 | 0.49 | 0.49 | 1.9 |
| LOQ | Conven- | 1.5 | 2.9 | 2.9 | 12 |
| (pg/ | tional | | | | |
| mL) | 10 μL | 1.5 | 2.9 | 2.9 | 23 |
| | 30 μL | 0.73 | 2.9 | 2.9 | 12 |
| | 90 μL | 0.18 | 0.73 | 0.73 | 5.9 |

As shown in Table 6, as a general trend, increasing the sample volume leads to an increase in the sensitivity (lower LOD and/or LOQ). A fact that distinguishes the assays is the level of improvement. For instance, the IL-4 assay shows an approximately 4 fold improvement in LOD and an approximately 8 fold improvement in LOQ when comparing the 10 μl and 90 μl sample volume data. The IFN-gamma assay on the other hand only shows approximately 2 fold and less than 4 fold improvements in LOD and LOQ respectively. This illustrates the fact that although most, if not all, assays will show sensitivity improvement; the gain should be established for individual assays on a case-by-case basis. In the manual mode the gains in LOQ improvement are partially limited by the precision (variance) caused by slight variations in operator performance. One example of this is slight variations in incubation times for each step for replicate runs.

This effect can be extended even further by increasing the number of repeat sample volume loads. As an illustrative experiment, Case Study 2 below summarizes the experimental protocol and results for an IL-6 assay with 270 μl sample volume. Unless otherwise noted the remainder of the protocol follows Case Study 1 format, and the same clone #'s as identified in Table 6 are used for this experiment.

### Case Study 2: Abbreviated Assay Protocol for 10 μl (Static) and 90 μl and 270 μl (Flow-Through) Run for IL-6

1) Assemble Optimiser™ plate with absorbent pad and holder. Prime the Optimiser™ plate with the PBS based priming buffer as described herein.
2) Add 10 μl of capture antibody solution into each well, and incubate at room temperature for 10 minutes.
3) Add 10 μl of blocking buffer into each well, and incubate at room temperature for 10 minutes.
4) For static mode: Prepare the standard solution with concentration in range of 2-500 pg/ml with zero, pipette 10 μl of each prepared standard solution into appropriate wells, and incubate at room temperature for 10 minutes*.
   For flow-through mode (90 μl): Prepare the standard solution with concentration in range of 0.4-100 pg/ml with zero, pipette 30 μl of each prepared standard solution into appropriate wells, wait for 10 minutes, repeat three times, 90 μl of total volume was loaded into each well.
   For flow-through mode (270 μl): Prepare the standard solution with concentration in range of 0.1-25 pg/ml with zero, pipette 30 μl of each prepared standard solution into appropriate wells, wait for 10 minutes, repeat nine times, 270 μl of total volume was loaded into each well.
5) Add 30 μl of wash buffer into each well, wait until all wells are empty.
6) Add 10 μl of detection antibody solution into each well, and incubate at room temperature for 5 minutes.
7) Repeat step 5.
8) Add 10 μl of SAv-HRP solution into each well, and incubate at room temperature for 5 minutes.
9) Repeat step 5, change the absorbent pad, repeat step 5 again.
10) Add 10 μl of QuantaRed working solution in each well, wait until all wells are empty, take off the plate from the holder, wipe off all residue from bottom of Optimiser™ plate with Kimwipe, measure the fluorescence at 15 minutes after adding substrate with wavelength at 528/590 nm and sensitivity at 45.

The comparative results for static mode (10 μl sample volume) and the so-called flow-through mode (90 μl and 270 μl sample volumes; or essentially any sample volume greater than 30 μl) are shown in the Table CS3 below and clearly show the huge sensitivity improvement. The following table shows Case Study 2 data for an IL-6 assay tested on the Optimiser™ microplate showing the effect of sample volume on the sensitivity of detection.

As explained previously, a source of limitation for the manual mode is the variance caused by deviations in a human operator's operation sequence. This is precisely the reason why large number of repeat loads are ideally suited for an automation based system. The experiments above were repeated on a BioTek Precision microplate dispenser and lead to startling improvements in performance. Since the automation system can dispense low volumes (˜1-5 μl) with good repeatability, the experiment was modified such that instead of dispensing 30 μl in a single load, the minimum volume required to fill the microchannel (with slight excess) which is approximately 5 μl was dispensed in each loading step. Each dispense cycle was then followed by a precisely timed incubation cycle. Hence, as shown below in Table 7, the total assay times for the 6× loading cycles and 20× cycles are higher than the single load 10 μl case. However, this demonstrates yet another “tuning” feature of the Optimiser™—namely that by extending the time of the assay, while still being significantly less than the total assay time of conventional 96-well microplates; the sensitivity gains are astounding.

As part of the optimization effort in the development of the present invention, the location of the dispensing tip when dispensing into the Optimiser™ was parametrically optimized. Since the automation system can repeatably load at the same exact location (tolerance ˜100 μm); this optimization step yields enhanced precision performance. Specifically, the position of the dispense tip was optimized such that the dispense tip always touched the loading well surface in close vicinity of the through hole; more specifically approximately at 250 μm (tolerance˜100 μm) radial distance on horizontal plane from edge of the through hole. These experiments also followed a similar format as the case study except for the exceptions in sample loading as described above. The results are shown below in Table 7—which clearly show that there is a tremendous gain in sensitivity when the automated system approach; i.e. low volume but higher repeat loads and longer overall incubation time (10 minutes for each step) for sample incubation step is used. The data in Table 7 clearly illustrates an interesting finding—namely that a single load of 30 μl is significantly less effective than multiple loads (5 μl×6 loads). This is logically consistent since the internal volume of the channel is only ˜5 μl and in the repeat load mode, each “load volume” is allowed to incubate for a sizeable incubation interval (5-25 minutes range; 10 minutes in this case) for maximum binding to the capture antibody already coated on the channel walls. On the other hand, when 30 μl volume is loaded in a single step; the residence time for an aliquot of sample within the microfluidic detection chamber is only ˜10 sec-1 minute as it is flowing through the channel. This is a significant finding that will allow for additional assay optimization techniques such as repeat loads of capture antibody; blocking buffer etc to ensure optimum assay performance.

TABLE
LOD/LOQ comparison for 10 μl, 5 μl × 6 (net 30 μl), and
5 μl × 20 (net 100 μl) sample volumes on Optimiser ™
using automated dispensing system

| | | Human | Mouse | Mouse |
|---|---|---|---|---|
| | | IL-4 | IL-2 | IL-17A |
| Capture | | 8D4-8 | JES6-1A12 | TC11-18H10.1 |
| antibody | | | | |
| Detection | | MP4-25D2 | JES6-5H4 | TC11-8H4 |
| antibody | | | | |
| Antigen | | eBio- | eBio- | eBio- |
| | | science, | science, | science, |
| | | 14-8049-62 | 14-8021-64 | 14-8171-62 |
| LOD | Conven- | 0.49 | 1.9 | 7.8 |
| (pg/ | tional | | | |
| mL) | 10 μL | 0.98 | 1.9 | 15 |
| | 5 μL × 6 | 0.12 | 0.49 | Not tested |
| | 5 μL × 20 | 0.031 | 0.12 | 0.49 |
| LOQ | Conven- | 1.5 | 2.9 | 23 |
| (pg/ | tional | | | |
| mL) | 10 μL | 1.5 | 2.9 | 23 |
| | 5 μL × 6 | 0.37 | 1.5 | Not tested |
| | 5 μL × 20 | 0.047 | 0.37 | 2.9 |

| | | | |
|---|---|---|---|
| Better in pre-wetting? | No | Yes | Yes |
| Incubation time* | 10 min | 10 min | 10 min |

### Case Study 3: Effect of pH of Coat Buffer on Optimiser™ Assay Performance

Assay screening with coating buffers at pH in range from 5.0 to 10.5.

Unlike the assay in conventional plate, the capture antibody adsorption in Optimiser™ is dominated by the reaction rate of protein adsorption, which is strongly affected by the ingredients of coating buffer. A coating buffer screening test with pH in range from 5.0 to 10.5 has been performed with various assays.

Coating buffer: Phosphate citrate buffer, pH at 5.0 and 5.5; PBS buffer, pH at 6.0, 6.5, 7.0, 7.5;
Tris buffer, pH at 8.0, 8.5, 9.0; and Carbonate-Bicarbonate buffer, pH at 9.5, 10.0, 10.5

Experiment: follow the standard protocol described previously, no priming step, dilute the capture antibody with buffers above, one wash step after capture antibody incubation, using one concentration for each antigen.

### Results:

Assay response profile with coating buffer at pH in range from 5.0 to 10.5.

Conclusion: All assays shows better dose response in pH range lower than 7.0.

Assay screening with Citric Acid-Na₂HPO₄ coating Buffer at pH in range from 2.8 to 7.2

Coating buffer: Citric Acid-Na₂HPO₄ buffer has wide buffer capability with pH range from 2.6-7.6. 24 types of Citric Acid-Na₂HPO₄ buffer were prepared with pH from 2.6-7.2. This is an extension of the test from CS3.1 for a more comprehensive screen.

Experiment: follow the standard protocol, no priming step, dilute the capture antibody with buffers above, one wash step after capture antibody incubation, use one concentration for each antigen.

Results:

Assay response profile with coating buffer at pH in range from 5.0 to 10.5.

TABLE
Optimal pH range for each assay

| | pH value that give strongest signal |
|---|---|
| Assay | (larger than 90% of maximum) |
| Mouse IL-2 | 4.6, 4.8, 5.0 |
| Human IL-6 | 5.6 |
| Human IL-4 | 4.0, 4.2, 4.4, 4.6 |
| Mouse IL-17A | 4.6, 4.8 |
| Mouse IFN-γ | 5.0, 5.2 |

### Conclusion and Discussion:

There is a range of optimal pH for each assay which gives strongest signal.
The window of the optimal pH range varies by assay as further described in Case Study 3; Appendix 2.

### Comparison Between PBS Buffer and Citric Acid-Na₂HPO₄ Buffer

Coating buffer: PBS buffers with pH range from 2.6-6.9 were prepared by pH adjusting with HCl solution. Note: PBS buffer is intended for use at pH lower than 6, it is only used for comparison study.

Experiment: follow the standard protocol, no priming step, dilute the capture antibody with buffers above, one wash step after capture antibody incubation, mouse IL-2 assay has been tested (at 100 pg/mL). Results are compared with assay results with Citric Acid-Na₂HPO₄ Buffer, shown in FIG. 3.

Assay response profile with PBS buffer and Citric Acid-Na₂HPO₄ buffer at pH in range from 2.6 to 7.2.

### Conclusion and Discussion:

The assay response profiles between PBS buffer and Citric Acid-Na₂HPO₄ Buffer are almost identical
This data establishes that pH (and not the composition of the coat buffer) is the dominant factor for assay response.
   Assay Comparison Between Optimiser™ and Conventional Plate with Citric Acid-Na₂HPO₄ Buffer

Coating buffer: 24 types of Citric Acid-Na₂HPO₄ buffer were prepared with pH from 2.6-7.2.

### Experiment:

1) For Optimiser™ assay, follow the standard protocol, no priming step, dilute the capture antibody with buffers above, one wash step after capture antibody incubation, only mouse IL-2 assay, 100 pg/mL mouse IL-2 has been tested.
2) For conventional plate, use NUNC MaxiSorp® high binding ELISA plate, follow the standard protocol, dilute the capture antibody with buffers above, only mouse IL-2 assay, 100 pg/mL mouse IL-2 has been tested.
   Assay Response Profile with Citric Acid-Na₂HPO₄ Buffer at pH in Range from 2.6 to 7.2, Comparison Study of Optimiser™ and Conventional Plate

### Conclusion and Discussion:

The effect of pH of coat buffer on the Optimiser™ based assays is much more significant that the effect of pH of coat buffer on conventional plate assays. This is a novel phenomenon and is significantly different from the effect of pH of coat buffers on assay performance as currently known in the art.
   Sensitivity Improvement in Optimiser™ with Citric Acid-Na₂HPO₄ Buffer

The sensitivity can be improved by using coating buffer with optimal pH with Optimiser™ plate. In most cases, Optimiser™ assay with only 10 μL of sample could give better sensitivity than conventional assay using same concentration of antibodies.

TABLE
LOD/LOQ comparison for Optimiser ™ assay with priming method and optimal coating buffer

| | | Human IL-4 | Human IL-6 | Mouse IL-2 | Mouse IFN-gamma | Mouse IL-17A |
|---|---|---|---|---|---|---|
| Capture antibody | | 8D4-8 | MQ2-13A5 | JES6-1A12 | AN-18 | TC11-18H10.1 |
| Detection antibody | | MP4-25D2 | MQ2-39C3 | JES6-5H4 | R4-6A2 | TC11-8H4 |
| Antigen | | eBioscience, | eBioscience, | eBioscience, | BD | eBioscience, |
| | | 14-8049-62 | 14-8069-62 | 14-8021-64 | Biosciences, | 14-8171-62 |
| | | | | | 554587 | |
| LOD | Conventional | 0.49 | 1.9 | 1.9 | 7.8 | 7.8 |
| (pg/mL) | Priming method | 0.98 | 0.98 | 1.9 | 15 | 15 |
| | Optimal coating | 0.19 | 0.98 | 0.58 | 2.3 | 1.2 |
| | buffer | | | | | |
| LOQ | Conventional | 1.5 | 2.9 | 2.9 | 12 | 23 |
| (pg/mL) | Priming method | 1.5 | 2.9 | 2.9 | 23 | 23 |
| | Optimal coating | 0.78 | 2.9 | 2.3 | 9.4 | 4.7 |
| | buffer | | | | | |

As an example, below is the comparison between IL-4 assay using PBS, pH 7.2 as coating buffer with priming step in assay sequence and same assay using Citric Acid-Na₂HPO₄ buffer at pH 4.4 without using a priming step.

Comparison Study of IL-4 Assay with Priming Method and Citric Acid-Na₂HPO₄ Buffer at pH 4.4

### Conclusion and Discussion:

The use of optimal coat buffer is a better technique for improving assay performance than the use of the priming step.
   Sensitivity Improvement in Optimiser™ with High Concentration of Capture Antibody:

With optimal coating buffer, most Optimiser™ assay will achieve same or better sensitivity than conventional assay with same antibody concentrations. Furthermore, large surface area and high surface area to volume ratio in the microfluidic channel of Optimiser™ plate allow more capture antibody adsorbed onto the surface comparing to conventional plate. Table below shows that some assays exhibit significant improvement in performance when higher concentrations of capture and/or detection antibody are used.

TABLE
LOD/LOQ comparison for Optimiser ™ assay with elevated capture antibody concentration

| | | Human IL-4 | Human IL-6 | Mouse IL-2 | Mouse IFN-gamma | Mouse IL-17A |
|---|---|---|---|---|---|---|
| Capture antibody | | 8D4-8 | MQ2-13A5 | JES6-1A12 | AN-18 | TC11-18H10.1 |
| Detection antibody | | MP4-25D2 | MQ2-39C3 | JES6-5H4 | R4-6A2 | TC11-8H4 |
| Antigen | | eBioscience, | eBioscience, | eBioscience, | BD | eBioscience, |
| | | 14-8049-62 | 14-8069-62 | 14-8021-64 | Biosciences, | 14-8171-62 |
| | | | | | 554587 | |
| LOD | Conventional | 0.49 | 1.9 | 1.9 | 7.8 | 7.8 |
| (pg/mL) | Optimiser ™ assay, same | 0.19 | 0.98 | 0.58 | 2.3 | 1.2 |
| | concentration of capture | | | | | |
| | antibody as used in | | | | | |
| | conventional | | | | | |
| | Optimiser ™ assay, 4 | 0.19 | 0.49 | 0.58 | 1.2 | 1.2 |
| | times of concentration | | | | | |
| | of capture antibody as | | | | | |
| | used in conventional | | | | | |
| | assay | | | | | |
| LOQ | Conventional | 1.5 | 2.9 | 2.9 | 12 | 23 |
| (pg/mL) | Optimiser ™ assay, same | 0.78 | 2.9 | 2.3 | 9.4 | 4.7 |
| | concentration of capture | | | | | |
| | antibody as used in | | | | | |
| | conventional | | | | | |
| | Optimiser ™ assay, 4 | 0.78 | 1.9 | 2.3 | 4.7 | 4.7 |
| | times of concentration | | | | | |
| | of capture antibody as | | | | | |
| | used in conventional | | | | | |
| | assay | | | | | |

In above 5 assays, using 4 times of concentration of capture antibody in Optimiser™ assay, the sensitivity of human IL-6 and mouse IFN-gamma assay is improved about two times.

Abbreviated assay protocol for 10 μl run for Optimiser™ assay with Optimal coating buffer:
1) Assemble Optimiser™ plate with absorbent pad and holder.
2) Prepare capture antibody with optimal coating buffer, add 10 μl of capture antibody solution into each well, and incubate at room temperature for 10 minutes.
3) Add 10 μl of wash buffer into each well, wait until all wells are empty.
4) Add 10 μl of blocking buffer into each well, and incubate at room temperature for 10 minutes.
5) Prepare the standard and sample solution, pipette 10 μl of each prepared solution into appropriate wells, and incubate at room temperature for 10 minutes.
6) Add 10 μl of detection antibody solution into each well, and incubate at room temperature for 5 minutes.
7) Repeat step 5.
8) Add 10 μl of SAv-HRP solution into each well, and incubate at room temperature for 5 minutes.
9) Repeat step 5, change the absorbent pad, repeat step 5 again.
10) Add 10 μl of QuantaRed working solution in each well, wait until all wells are empty, take off the plate from the holder, wipe off all residue from bottom of Optimiser™ plate with Kimwipe, measure the fluorescence at 15 minutes after adding substrate with wavelength at 528/590 nm.

### Effect of Coating Buffers on Various Assays:

The Tables below show that the coating buffer (OptiBind™) choice is widely different for different assays and even when multiple assays share the same OptiBind™ formulation as ideal coat buffer, the range of alternate buffers is different. Note that the various OptiBind™ formulations are identified by letters A through L with A corresponding to a pH 2.8 buffer, B corresponding to a pH 3.2 buffer, C corresponding to a pH 3.6 buffer and so on with L corresponding to a pH 7.2 buffer as listed in Table CS3.4.

TABLE
OptiBind ™ Coating buffer formulations
and the pH of each formulation

| A | B | C | D | E | F | G | H | I | J | K | L |
|---|---|---|---|---|---|---|---|---|---|---|---|
| 2.8 | 3.2 | 3.6 | 4.0 | 4.4 | 4.8 | 5.2 | 5.6 | 6.0 | 6.4 | 6.8 | 7.2 |

TABLE
Ideal coat buffer for various assays with capture antibodies with
specific clone numbers. The “Best” coat buffer is the one that
yields maximum signal and the “Alternate” coat buffers are
ones that show at least 90% sensitivity as compared to “Best”.

| | | BEST | Alternate |
|---|---|---|---|
| | CLONE NO. | Coat Buffer | Coat Buffer |
| ANALYTE | (Capture | (OPTI- | (OPTI- |
| NAME | Antibody) | BIND ™) | BIND ™) |
| Human IL-4 | 8D4-8 | E | D |
| Human IL-17AF | 4H450 + | E | D |
| | 4H1420 | | |
| Human IL-23 | 6H617 | E | D |
| Mouse IL-2 | JES6-1A12 | F | None |
| Mouse IL-17A | TC11-18H10.1 | F | None |
| Mouse IFN-gamma | AN-18 | G | None |
| Human MIP3a | 8B1.1D3 | G | None |
| Human IL-17A | 4H152 | G | H |
| Human IL-6 | MQ2-13A5 | H | None |

TABLE
Ideal coat buffer for various assays with capture antibodies specified
by vendor catalog numbers. The “Best” coat buffer is the one that
yields maximum signal and the “Alternate” coat buffers are
ones that show at least 90% sensitivity as compared to “Best”.

| | | | BEST | Alternate |
|---|---|---|---|---|
| | | | Coat | Coat |
| | VENDOR for | CATA- | Buffer | Buffer |
| ANALYTE | Capture | LOGUE | (OPTI- | (OPTI- |
| NAME | Antibody | NO. | BIND ™) | BIND ™) |
| Human HGF | R&D SYSTEMS | DY294 | D | E |
| Human G-CSF | R&D SYSTEMS | DY214 | D | None |
| Human MIF | R&D SYSTEMS | DY289 | E | None |
| Human Leptin | R&D SYSTEMS | DY398 | E | D |
| Troponin I | Hytest | 4T21 | E | F |
| | | MAb19c7 | | |
| Human TGF β1 | R&D SYSTEMS | DY240 | E | A, B, C, |
| | | | | D |
| Human IL-13 | abcam | ab47447 | E | None |
| Human IFN-γ | R&D SYSTEMS | DY285 | E | None |
| Human IL-18 | e-Bioscience | BMS267/ | E | F, G, H |
| | | 2MST | | |
| Human IL-23 | R&D SYSTEMS | DY1290 | E | None |
| Human HB EGF | R&D SYSTEMS | DY258 | E | F |
| Human TGF β1 | Invitrogen | CHC1683 | F | E, G |
| Human VEGF | Invitrogen | CHG0113 | F | G |
| Human Prolactin | R&D SYSTEMS | DY682 | F | E |
| Human IGF-II | R&D SYSTEMS | MAB2921 | F | None |
| Human CA125 | abcam | ab10029 | F | G |
| Mouse IL-4 | Invitrogen | CMC0043 | F | E, G |
| Gen assay#2 | Genentech | N/A | F | None |
| Mouse TNF-α | R&D SYSTEMS | DY410 | F | E |
| Human IL-10 | ABDSerotec | MCA1531 | F | None |
| AKT[pS473] | Invitrogen | CHO0115 | G | H |
| Gen assay#1 | Genentech | N/A | G | D, E, F, |
| | | | | H |
| Human c-Met | Invitrogen | CHO0285 | H | F, G |
| (Total) | | | | |
| Mouse IL-10 | BD Pharmingen | 551215 | H | None |
| Human | R&D SYSTEMS | DY1433 | H | G |
| Osteopontin | | | | |
| Mouse IL-10 | R&D SYSTEMS | DY417 | H | I, J, K |
| Human IL-1α/ | R&D SYSTEMS | DY200 | H | F, G, I |
| IL1-F | | | | |
| Human IL-6 | R&D SYSTEMS | DY206 | H | E, F, G |
| Human TNF-α/ | R&D SYSTEMS | DY210 | H | F, G, I |
| TNFSF1 | | | | |
| Human IL-12p 70 | R&D SYSTEMS | DY1270 | H | G, I |
| Mouse IL-6 | Invitrogen | CMC0063 | J | H, I, K |
| Mouse TNF-α | Invitrogen | CMC3013 | K | None |
| Human IFN-α | MABTECH | 3423-1H-6 | L | I, J |

While the present invention has been described in detail herein in various preferred embodiments, it will be apparent to those skilled in the art that various modifications or variations may be made to the preferred embodiments and variations of the invention as described herein without departing in any way from the spirit and scope of the invention. Accordingly, all such modifications and variations are intended to be incorporated herein and within the scope of this invention, which is intended to be defined solely by the appended claims.

What is claimed is:

1. In an improved method for performing an immunoassay or group of immunoassays on a sample on a selected microfluidic microplate, wherein a priming buffer is used as the first reagent in the immunoassay sequence, the improvement wherein the priming buffer is a liquid with lower surface tension than the surface tension of water.

2. The improved method of claim 1, wherein the priming buffer is selected from the group consisting of alcohols, aqueous solutions with high protein content, and other solvents.

3. The improved method of claim 1, wherein the priming buffer is an aqueous buffer.

4. The improved method of claim 1, wherein the microfluidic microplates are pre-coated with a biomolecule using a priming liquid with lower surface tension than the surface tension of water.

5. The improved method of claim 1, wherein the priming buffer is an aqueous buffer selected from the group consisting of Phosphate Buffer Solution (PBS) and Tris Buffer Solution (TBS).

6. A method for increasing the sensitivity of immunoassays performed using microfluidic microplates, which method comprises using suitably high concentrations of capture and/or detection antibodies as compared to the concentrations of capture and/or detection antibody concentrations, and wherein the capture and/or detection antibody concentration is greater than and up to at least 20 times higher than the concentration of the capture and/or detection antibody used for the same assay on a conventional 96-well microplate.

7. The improved method of claim 1, wherein repeated additions of sample of up to approximately 5 microliter aliquots of sample are added and allowed to incubate for from approximately 1 minute to approximately 20 minutes for each aliquot.

8. The improved method of claim 1, wherein the method further comprises the steps of selecting a coating buffer with optimal pH wherein the optimal pH is within a range of from less than about plus or minus 0.4 pH value to about 1 pH value from the optimal pH for the coat buffer for a particular capture antibody for a given assay.

9. The improved method of claim 8, wherein each assay or group of assays uses a different coating buffer and further wherein the range of the optimal pH of the coating buffer is different for each assay or group of assays.

10. The improved method of claim 8, wherein the assays are selected from the group consisting of Human IL-4 using capture antibody clone 8D4-8; Human IL17-AF using capture antibody clone 4H450 and 4H1420, Human IL-23 using capture antibody clone 6H617, Mouse IL-2 using capture antibody JES6-1A12, Mouse IL-17A using capture antibody clone TC11-18H10.1, Mouse IFN-gamma using capture antibody AN-18, Human MIP3-alpha using capture antibody clone 8B1.1D3, Human IL-17A using capture antibody clone 4H152, and Human IL-6 using capture antibody clone MQ2-13A5.
